SEQUENCE LISTING (757 sequences)

SEQ ID NO: 1 - protein, 15 aa, Mycobacterium tuberculosis
TDAATLAQEAGNFER

SEQ ID NO: 2 - protein, 18 aa, Mycobacterium tuberculosis
TQIDQVESTAGSLQGQWR

SEQ ID NO: 3 - protein, 17 aa, Mycobacterium tuberculosis
WDATATELNNALQNLAR

SEQ ID NO: 4 - protein, 18 aa, Mycobacterium tuberculosis
TQIDQVESTAASLQAQWR

SEQ ID NO: 5 - protein, 12 aa, Human immunodeficiency virus 1
ETINEEAAEWDR

SEQ ID NO: 6 - protein, 12 aa, Human immunodeficiency virus 1
DTINEEAAEWDR

SEQ ID NO: 7 - protein, 11 aa, Human immunodeficiency virus 1
MYSPTSILDIR

SEQ ID NO: 8 - protein, 11 aa, Human immunodeficiency virus 1
MYSPVSILDIK

SEQ ID NO: 9 - protein, 11 aa, Human immunodeficiency virus 1
MYSPVSILDIR

SEQ ID NO: 10 - protein, 11 aa, Human immunodeficiency virus 2
MYNPTNILDIK

SEQ ID NO: 11 - protein, 9 aa, Human immunodeficiency virus 2
AEQTDPAVK

SEQ ID NO: 12 - protein, 12 aa, Human immunodeficiency virus 2
MYNPPTNILDIK

SEQ ID NO: 13 - protein, 12 aa, Human immunodeficiency virus 1
ETINEEAAEWER

SEQ ID NO: 14 - protein, 15 aa, Human immunodeficiency virus 1
VHPVHAGPIPPGQMR

SEQ ID NO: 15 - protein, 12 aa, Human immunodeficiency virus 1
  VARIANT 1: note = Glu or Asp
XTINEEAAEWDR

SEQ ID NO: 16 - protein, 11 aa, Human immunodeficiency virus 1
  VARIANT 11: note = Arg or Lys
MYSPTSILDIX

SEQ ID NO: 17 - protein, 11 aa, Ebola virus
LGPGIPDHPLR

SEQ ID NO: 18 - protein, 11 aa, Ebola virus
LGQGIPDHPLR

SEQ ID NO: 19 - protein, 9 aa, Ebola virus
LRPILLPNK

SEQ ID NO: 20 - protein, 9 aa, Ebola virus
LRPILLPGK

SEQ ID NO: 21 - protein, 9 aa, Ebola virus
LRPILLPGR

SEQ ID NO: 22 - protein, 9 aa, Ebola virus
LRPVLLPGK

SEQ ID NO: 23 - protein, 11 aa, Human immunodeficiency virus 2
MYNPTNILDIK

SEQ ID NO: 24 - protein, 11 aa, Mycobacterium tuberculosis
LYASAEATDSK

SEQ ID NO: 25 - protein, 9 aa, Mycobacterium tuberculosis
NDPTQQIPK

SEQ ID NO: 26 - protein, 15 aa, Mycobacterium tuberculosis
AQAASLEAEHQAIVR

SEQ ID NO: 27 - protein, 11 aa, Mycobacterium tuberculosis
FLSAATSSTPR

SEQ ID NO: 28 - protein, 7 aa, Mycobacterium tuberculosis
VEYVDVR

SEQ ID NO: 29 - protein, 15 aa, Mycobacterium tuberculosis
IESENPDAVANVQAR

SEQ ID NO: 30 - protein, 12 aa, Mycobacterium tuberculosis
GVTEETTTGVLR

SEQ ID NO: 31 - protein, 13 aa, Mycobacterium tuberculosis
LEEENPEAAQALR

SEQ ID NO: 32 - protein, 12 aa, Mycobacterium tuberculosis
NQAETLVYQTEK

SEQ ID NO: 33 - protein, 14 aa, Mycobacterium tuberculosis
GSLVEGGIGGTEAR

SEQ ID NO: 34 - protein, 15 aa, Mycobacterium tuberculosis
AQAGLLEAEHQAIVR

SEQ ID NO: 35 - protein, 12 aa, Mycobacterium tuberculosis
SLADPNVSFANK

SEQ ID NO: 36 - protein, 10 aa, Mycobacterium tuberculosis
IALFGNHAPK

SEQ ID NO: 37 - protein, 11 aa, Mycobacterium tuberculosis
QELDEISTNIR

SEQ ID NO: 38 - protein, 10 aa, Mycobacterium tuberculosis
SLENYIAQTR

SEQ ID NO: 39 - protein, 19 aa, Mycobacterium tuberculosis
TTGDPPFPGQPPPVANDTR

SEQ ID NO: 40 - protein, 10 aa, Mycobacterium tuberculosis
IPDEDLAGLR

SEQ ID NO: 41 - protein, 13 aa, Mycobacterium tuberculosis
TVANFVGLAQGTK

SEQ ID NO: 42 - protein, 8 aa, Mycobacterium tuberculosis
LVFLTGPK

SEQ ID NO: 43 - protein, 9 aa, Mycobacterium tuberculosis
MPAVTDLVK

SEQ ID NO: 44 - protein, 9 aa, Mycobacterium tuberculosis
AFDWDQAYR

SEQ ID NO: 45 - protein, 11 aa, Mycobacterium tuberculosis
TVGDVVAYIQK

SEQ ID NO: 46 - protein, 15 aa, Mycobacterium tuberculosis
TDAATLAQEAGNFER

SEQ ID NO: 47 - protein, 8 aa, Mycobacterium tuberculosis
VVDWLVDK

SEQ ID NO: 48 - protein, 18 aa, Mycobacterium tuberculosis
TQIDQVESTAGSLQGQWR

SEQ ID NO: 49 - protein, 15 aa, Mycobacterium tuberculosis
AADMWGPSSDPAWER

SEQ ID NO: 50 - protein, 15 aa, Mycobacterium tuberculosis
IPLDVAEGDTVIYSK

SEQ ID NO: 51 - protein, 31 aa, Mycobacterium tuberculosis
FSDPSKPNGQIWTGVIGSPAANAPDAGPPQR

SEQ ID NO: 52 - protein, 12 aa, Mycobacterium tuberculosis
FLLDQAITSAGR

SEQ ID NO: 53 - protein, 20 aa, Mycobacterium tuberculosis
EAPYELNITSATYQSAIPPR

SEQ ID NO: 54 - protein, 20 aa, Mycobacterium tuberculosis
GFQSIHESDMLLLPDPETAR

SEQ ID NO: 55 - protein, 15 aa, Mycobacterium tuberculosis
SVFDDGLAFDGSSIR

SEQ ID NO: 56 - protein, 17 aa, Mycobacterium tuberculosis
PGLPVEYLQVPSPSMGR

SEQ ID NO: 57 - protein, 17 aa, Mycobacterium tuberculosis
WDATATELNNALQNLAR

SEQ ID NO: 58 - protein, 14 aa, Mycobacterium tuberculosis
AGANLFELENFVAR

SEQ ID NO: 59 - protein, 17 aa, Mycobacterium tuberculosis
WETFLTSELPGWLQANR

SEQ ID NO: 60 - protein, 31 aa, Mycobacterium tuberculosis
DPASAPDVPTAAQLTSLLNSLADPNVSFANK

SEQ ID NO: 61 - protein, 14 aa, Mycobacterium tuberculosis
TNLTTEQIANYVAR

SEQ ID NO: 62 - protein, 21 aa, Mycobacterium tuberculosis
DPALCQHKPLTPQGDELSKPR

SEQ ID NO: 63 - protein, 15 aa, Mycobacterium tuberculosis
LGGLSAPPWAKPEDR

SEQ ID NO: 64 - protein, 15 aa, Mycobacterium tuberculosis
DLEVETLTASSEGNK

SEQ ID NO: 65 - protein, 16 aa, Mycobacterium tuberculosis
TTLPGVVNGANNPAIR

SEQ ID NO: 66 - protein, 15 aa, Mycobacterium tuberculosis
YSDIEPSTGGEVVLK

SEQ ID NO: 67 - protein, 13 aa, Mycobacterium tuberculosis
QYHVQFFGDAPER

SEQ ID NO: 68 - protein, 14 aa, Mycobacterium tuberculosis
MTSEIETNIVAVER

SEQ ID NO: 69 - protein, 17 aa, Mycobacterium tuberculosis
MEPGPDGPAASGPAAIR

SEQ ID NO: 70 - protein, 22 aa, Mycobacterium tuberculosis
VNDPTESQQEDQLIAGAQDEAK

SEQ ID NO: 71 - protein, 14 aa, Mycobacterium tuberculosis
EQVQIGAHSPPQFR

SEQ ID NO: 72 - protein, 14 aa, Mycobacterium tuberculosis
TEQEVVEGMDISTR

SEQ ID NO: 73 - protein, 13 aa, Mycobacterium tuberculosis
VVCTESWPLAHHR

SEQ ID NO: 74 - protein, 16 aa, Mycobacterium tuberculosis
EPGPIAPSTNSSPVLK

SEQ ID NO: 75 - protein, 14 aa, Mycobacterium tuberculosis
AHFNAMFQPSSPTR

SEQ ID NO: 76 - protein, 14 aa, Mycobacterium tuberculosis
MDQFGNGLEIDQAR

SEQ ID NO: 77 - protein, 21 aa, Mycobacterium tuberculosis
DPALCQHKPLTPQGDELSEPR

SEQ ID NO: 78 - protein, 25 aa, Mycobacterium tuberculosis
METRPTALMSSTVAAAAPAAGAASR

SEQ ID NO: 79 - protein, 14 aa, Mycobacterium tuberculosis
TRPTDLVFVVDSSR

SEQ ID NO: 80 - protein, 15 aa, Mycobacterium tuberculosis
ELGFVQPSGVTDGMR

SEQ ID NO: 81 - protein, 13 aa, Mycobacterium tuberculosis
MLTDPDLPQEFER

SEQ ID NO: 82 - protein, 13 aa, Mycobacterium tuberculosis
CSTPLLHQQYTSR

SEQ ID NO: 83 - protein, 15 aa, Mycobacterium tuberculosis
ISCGPPAHVENAIAR

SEQ ID NO: 84 - protein, 17 aa, Mycobacterium tuberculosis
LLEAVGSSSGTPNAPPP

SEQ ID NO: 85 - protein, 22 aa, Mycobacterium tuberculosis
TPTQHSPVPPEEVTGPSQMDTR

SEQ ID NO: 86 - protein, 15 aa, Mycobacterium tuberculosis
TTYVSQSGQVISAPR

SEQ ID NO: 87 - protein, 14 aa, Mycobacterium tuberculosis
SFPQSSQLSQETVR

SEQ ID NO: 88 - protein, 15 aa, Mycobacterium tuberculosis
MNGDHMVLGSSVTDK

SEQ ID NO: 89 - protein, 12 aa, Mycobacterium tuberculosis
EYPLYINQTCHR

SEQ ID NO: 90 - protein, 15 aa, Mycobacterium tuberculosis
GEGAIGSLDYTPEER

SEQ ID NO: 91 - protein, 15 aa, Mycobacterium tuberculosis
DVVHPLGGEEPSMAR

SEQ ID NO: 92 - protein, 13 aa, Mycobacterium tuberculosis
VYQPFLTTCDGHR

SEQ ID NO: 93 - protein, 13 aa, Mycobacterium tuberculosis
AFDCPSSFQIHER

SEQ ID NO: 94 - protein, 15 aa, Mycobacterium tuberculosis
TLAPQVCSSFATGPR

SEQ ID NO: 95 - protein, 21 aa, Mycobacterium tuberculosis
SEPCDDLQIPNTNVHLSHDAK

SEQ ID NO: 96 - protein, 13 aa, Mycobacterium tuberculosis
FNNVQLNLTDEER

SEQ ID NO: 97 - protein, 15 aa, Mycobacterium tuberculosis
QLVDEFQASGGVGER

SEQ ID NO: 98 - protein, 23 aa, Mycobacterium tuberculosis
EDASGQLSCIQLPVDSQGGDANK

SEQ ID NO: 99 - protein, 13 aa, Mycobacterium tuberculosis
FTSDMSNTEWGYR

SEQ ID NO: 100 - protein, 14 aa, Mycobacterium tuberculosis
MAFMAATDHSDQLR

SEQ ID NO: 101 - protein, 15 aa, Mycobacterium tuberculosis
LEIGPVYSSVSSEAR

SEQ ID NO: 102 - protein, 14 aa, Mycobacterium tuberculosis
TIGMPATEEVDCIR

SEQ ID NO: 103 - protein, 14 aa, Mycobacterium tuberculosis
LLETVEYNISGAER

SEQ ID NO: 104 - protein, 14 aa, Mycobacterium tuberculosis
SQGDNNVSLVEEFR

SEQ ID NO: 105 - protein, 15 aa, Mycobacterium tuberculosis
TIQAPTQVPVVVSPR

SEQ ID NO: 106 - protein, 14 aa, Mycobacterium tuberculosis
CTLHLGIEPPDSVR

SEQ ID NO: 107 - protein, 23 aa, Mycobacterium tuberculosis
SEITNQLSVSDINSQSVGGARPK

SEQ ID NO: 108 - protein, 24 aa, Mycobacterium tuberculosis
WAGGPPGTGGHGPLSLNSPDPYEK

SEQ ID NO: 109 - protein, 14 aa, Mycobacterium tuberculosis
GCFTPVVTDPITER

SEQ ID NO: 110 - protein, 14 aa, Mycobacterium tuberculosis
GSIDDVFNCNLSPR

SEQ ID NO: 111 - protein, 22 aa, Mycobacterium tuberculosis
TFISLSSTDVSPNQSNTSNEMK

SEQ ID NO: 112 - protein, 21 aa, Mycobacterium tuberculosis
HVLTSHIDEPPTQNQSDLLNK

SEQ ID NO: 113 - protein, 16 aa, Mycobacterium tuberculosis
STLGPALEAVSMDGDK

SEQ ID NO: 114 - protein, 13 aa, Mycobacterium tuberculosis
VWTHCQTQHGIVK

SEQ ID NO: 115 - protein, 15 aa, Mycobacterium tuberculosis
QSGQCLDGVSLSSPR

SEQ ID NO: 116 - protein, 14 aa, Mycobacterium tuberculosis
ENYGSITSMGYESR

SEQ ID NO: 117 - protein, 14 aa, Mycobacterium tuberculosis
EGDYIVSVNGQPCR

SEQ ID NO: 118 - protein, 15 aa, Mycobacterium tuberculosis
EGYLQIGANTQAAQK

SEQ ID NO: 119 - protein, 14 aa, Mycobacterium tuberculosis
TNGVPTTEEVDCIR

SEQ ID NO: 120 - protein, 15 aa, Mycobacterium tuberculosis
VQTDKPHLVSLGSGR

SEQ ID NO: 121 - protein, 13 aa, Mycobacterium tuberculosis
YSSNLGNFNYEQR

SEQ ID NO: 122 - protein, 22 aa, Mycobacterium tuberculosis
VVAGDHNLSQNDGTEQYVSVQK

SEQ ID NO: 123 - protein, 13 aa, Mycobacterium tuberculosis
ILDETQEAVEYQR

SEQ ID NO: 124 - protein, 23 aa, Mycobacterium tuberculosis
DDSCSGDSSAQLSSGEHLLGPNR

SEQ ID NO: 125 - protein, 14 aa, Mycobacterium tuberculosis
SVQWCAVSQPEATK

SEQ ID NO: 126 - protein, 14 aa, Mycobacterium tuberculosis
NTSSEQEEVVEALR

SEQ ID NO: 127 - protein, 13 aa, Mycobacterium tuberculosis
TTTWQRPTMESVR

SEQ ID NO: 128 - protein, 16 aa, Mycobacterium tuberculosis
TASESISNLSEAGSIK

SEQ ID NO: 129 - protein, 15 aa, Mycobacterium tuberculosis
TPSTTTSSHYLGTLK

SEQ ID NO: 130 - protein, 14 aa, Mycobacterium tuberculosis
TVGFNHLTLGHNQR

SEQ ID NO: 131 - protein, 14 aa, Mycobacterium tuberculosis
GGHINDAFMTEDER

SEQ ID NO: 132 - protein, 13 aa, Mycobacterium tuberculosis
GFGCCFPCCSVDK

SEQ ID NO: 133 - protein, 13 aa, Mycobacterium tuberculosis
WTCSKPKPSTMLR

SEQ ID NO: 134 - protein, 14 aa, Mycobacterium tuberculosis
MPAYHSSLMDPDTK

SEQ ID NO: 135 - protein, 23 aa, Mycobacterium tuberculosis
SSLGLDNSLSTSSEDPHSGCPSR

SEQ ID NO: 136 - protein, 23 aa, Mycobacterium tuberculosis
GSECWHLSSGSVHPSPGSAPAQR

SEQ ID NO: 137 - protein, 15 aa, Mycobacterium tuberculosis
CQPLGSALPPQAPTR

SEQ ID NO: 138 - protein, 13 aa, Mycobacterium tuberculosis
NIQHLNSQIHSFR

SEQ ID NO: 139 - protein, 15 aa, Mycobacterium tuberculosis
VTTSDEDIGINAISR

SEQ ID NO: 140 - protein, 12 aa, Mycobacterium tuberculosis
FVELQVCDHYQR

SEQ ID NO: 141 - protein, 20 aa, Mycobacterium tuberculosis
HFITSSSSKPCEPEEHYVQK

SEQ ID NO: 142 - protein, 14 aa, Mycobacterium tuberculosis
TTGSTQSNFNFYVK

SEQ ID NO: 143 - protein, 24 aa, Mycobacterium tuberculosis
GLHGAATVVLGQGQHGGCAPEEED

SEQ ID NO: 144 - protein, 13 aa, Mycobacterium tuberculosis
TLTAEEAEEEWER

SEQ ID NO: 145 - protein, 15 aa, Mycobacterium tuberculosis
FGGTPIHFPGGRPPR

SEQ ID NO: 146 - protein, 14 aa, Mycobacterium tuberculosis
TLDENSDSAGLWQR

SEQ ID NO: 147 - protein, 29 aa, Mycobacterium tuberculosis
LNVAGAGGGGDSGGGPGAATDGNFTAGLA

SEQ ID NO: 148 - protein, 13 aa, Mycobacterium tuberculosis
VFSQNAYLIDHQR

SEQ ID NO: 149 - protein, 16 aa, Mycobacterium tuberculosis
EGSGNPTPLINPLAGR

SEQ ID NO: 150 - protein, 13 aa, Mycobacterium tuberculosis
TFAQTTYLIDHQR

SEQ ID NO: 151 - protein, 15 aa, Mycobacterium tuberculosis
VASMAPVTAEGFQER

SEQ ID NO: 152 - protein, 16 aa, Mycobacterium tuberculosis
SVLPPDGNGSPVLPDK

SEQ ID NO: 153 - protein, 14 aa, Mycobacterium tuberculosis
VNPDLQVEVKPSIR

SEQ ID NO: 154 - protein, 21 aa, Mycobacterium tuberculosis
TSETNTPQGNQEQLVTVMEER

SEQ ID NO: 155 - protein, 14 aa, Mycobacterium tuberculosis
QIQGTETEFNSLVK

SEQ ID NO: 156 - protein, 13 aa, Mycobacterium tuberculosis
DEIYIPLQEEDTK

SEQ ID NO: 157 - protein, 13 aa, Mycobacterium tuberculosis
VEDESLDNTWLNR

SEQ ID NO: 158 - protein, 22 aa, Mycobacterium tuberculosis
CVPRPGGAVCECPGGFQLDASR

SEQ ID NO: 159 - protein, 16 aa, Mycobacterium tuberculosis
MGGGGALQWNCSGGIQ

SEQ ID NO: 160 - protein, 22 aa, Mycobacterium tuberculosis
DVEEKPAHAPARPEAPVDSMLK

SEQ ID NO: 161 - protein, 16 aa, Mycobacterium tuberculosis
MAGSYPEGAPAILADK

SEQ ID NO: 162 - protein, 23 aa, Mycobacterium tuberculosis
LDCASAIQNYLSGTHGGSPGPER

SEQ ID NO: 163 - protein, 15 aa, Mycobacterium tuberculosis
HALPSPLEGSFQPGR

SEQ ID NO: 164 - protein, 14 aa, Mycobacterium tuberculosis
HCPSTFFSSPGLTR

SEQ ID NO: 165 - protein, 14 aa, Mycobacterium tuberculosis
ISDDTPLEMMTSPR

SEQ ID NO: 166 - protein, 19 aa, Mycobacterium tuberculosis
ELCDCEQCGEVFSEHSCLK

SEQ ID NO: 167 - protein, 15 aa, Mycobacterium tuberculosis
EGVYEISLSPTGVSR

SEQ ID NO: 168 - protein, 14 aa, Mycobacterium tuberculosis
SESEVHFDVETAIK

SEQ ID NO: 169 - protein, 14 aa, Mycobacterium tuberculosis
LIGTNCIIYPVNSK

SEQ ID NO: 170 - protein, 17 aa, Mycobacterium tuberculosis
CDVVVVGGGISGMAAAK

SEQ ID NO: 171 - protein, 14 aa, Mycobacterium tuberculosis
EEVYIVQASNVDVK

SEQ ID NO: 172 - protein, 24 aa, Mycobacterium tuberculosis
QEAASTGPGMEPETTATTILASVK

SEQ ID NO: 173 - protein, 14 aa, Mycobacterium tuberculosis
VVETMQSTLDAEIR

SEQ ID NO: 174 - protein, 14 aa, Mycobacterium tuberculosis
INSELQVPPTQVLR

SEQ ID NO: 175 - protein, 23 aa, Mycobacterium tuberculosis
QQNAQGGFSSTQDTVVALHALSK

SEQ ID NO: 176 - protein, 14 aa, Mycobacterium tuberculosis
FPSLLTQNENMVAK

SEQ ID NO: 177 - protein, 21 aa, Mycobacterium tuberculosis
QHHHQSSFPGSLPQETNLTLK

SEQ ID NO: 178 - protein, 24 aa, Mycobacterium tuberculosis
SGGEALAVANDSTSTPQNANGLWK

SEQ ID NO: 179 - protein, 13 aa, Mycobacterium tuberculosis
SSCSDMDLLHSWR

SEQ ID NO: 180 - protein, 14 aa, Mycobacterium tuberculosis
AGSVHYGHYTALCR

SEQ ID NO: 181 - protein, 14 aa, Mycobacterium tuberculosis
CNTQAELLAAGCQR

SEQ ID NO: 182 - protein, 13 aa, Mycobacterium tuberculosis
GVLVCDECCSVHR

SEQ ID NO: 183 - protein, 13 aa, Mycobacterium tuberculosis
TFMDMDQDSEDEK

SEQ ID NO: 184 - protein, 13 aa, Mycobacterium tuberculosis
LTWHSYPSEDDDK

SEQ ID NO: 185 - protein, 15 aa, Mycobacterium tuberculosis
FAVVENNSSAVTAQR

SEQ ID NO: 186 - protein, 14 aa, Mycobacterium tuberculosis
VEDTCVEWDPTGGK

SEQ ID NO: 187 - protein, 13 aa, Mycobacterium tuberculosis
AFSQNSQFIQHQR

SEQ ID NO: 188 - protein, 16 aa, Mycobacterium tuberculosis
CLPNPTPEGGAVPGPK

SEQ ID NO: 189 - protein, 14 aa, Mycobacterium tuberculosis
GEGQGLVCDLCNDR

SEQ ID NO: 190 - protein, 15 aa, Mycobacterium tuberculosis
SSTNTSLPDDNGAWK

SEQ ID NO: 191 - protein, 15 aa, Mycobacterium tuberculosis
LGEVVNTHGPVEPDK

SEQ ID NO: 192 - protein, 22 aa, Mycobacterium tuberculosis
QSSNRPAHNISHILGHDCSSAV

SEQ ID NO: 193 - protein, 27 aa, Mycobacterium tuberculosis
GPQGPTGSEGTPGLPGGVGQPGAVGEK

SEQ ID NO: 194 - protein, 23 aa, Mycobacterium tuberculosis
QGTLSTAAPTTSPAPCLSNHHNK

SEQ ID NO: 195 - protein, 14 aa, Mycobacterium tuberculosis
ENSENTTAPEVFPR

SEQ ID NO: 196 - protein, 15 aa, Mycobacterium tuberculosis
EQQMVPGIPQGAHEA

SEQ ID NO: 197 - protein, 14 aa, Mycobacterium tuberculosis
LVAACPESCVVCTK

SEQ ID NO: 198 - protein, 15 aa, Mycobacterium tuberculosis
ASCGQDQAAAETLLR

SEQ ID NO: 199 - protein, 14 aa, Mycobacterium tuberculosis
SMNDISLTPNTDQR

SEQ ID NO: 200 - protein, 24 aa, Mycobacterium tuberculosis
EDGTVSTANQNGVSSNGPGEILNK

SEQ ID NO: 201 - protein, 13 aa, Mycobacterium tuberculosis
SNPEDQILYQTER

SEQ ID NO: 202 - protein, 14 aa, Mycobacterium tuberculosis
LEGMNETVSNLTQR

SEQ ID NO: 203 - protein, 14 aa, Mycobacterium tuberculosis
KPSSETDIENWASK

SEQ ID NO: 204 - protein, 16 aa, Mycobacterium tuberculosis
MSESLDTADPAVTGAK

SEQ ID NO: 205 - protein, 13 aa, Mycobacterium tuberculosis
VTLYECHSQGEIR

SEQ ID NO: 206 - protein, 15 aa, Mycobacterium tuberculosis
VHSHEVAAYLASPGR

SEQ ID NO: 207 - protein, 15 aa, Mycobacterium tuberculosis
AGCQVVAPSDMMDGR

SEQ ID NO: 208 - protein, 23 aa, Mycobacterium tuberculosis
SISGTSTSEKPNSMDTANTSPFK

SEQ ID NO: 209 - protein, 15 aa, Mycobacterium tuberculosis
NFQSESVPALGGQEK

SEQ ID NO: 210 - protein, 14 aa, Mycobacterium tuberculosis
DSPVCPSYSPTMPR

SEQ ID NO: 211 - protein, 13 aa, Mycobacterium tuberculosis
DRPSLPQERPGWR

SEQ ID NO: 212 - protein, 21 aa, Mycobacterium tuberculosis
EDGEVVQEEEVCAKPSVTEEK

SEQ ID NO: 213 - protein, 14 aa, Mycobacterium tuberculosis
DAETGEEVTHYLVK

SEQ ID NO: 214 - protein, 15 aa, Mycobacterium tuberculosis
TGSLPHSSEQLLGHK

SEQ ID NO: 215 - protein, 14 aa, Mycobacterium tuberculosis
ASSVLPEHHEAFNR

SEQ ID NO: 216 - protein, 22 aa, Mycobacterium tuberculosis
LSGAMCTSCASQTTANDPYTVR

SEQ ID NO: 217 - protein, 14 aa, Mycobacterium tuberculosis
VSTAQDVIQQTLCK

SEQ ID NO: 218 - protein, 15 aa, Mycobacterium tuberculosis
VSSSSESEPELAQLK

SEQ ID NO: 219 - protein, 20 aa, Mycobacterium tuberculosis
TSCSNCTSNGMECMWCSSTK

SEQ ID NO: 220 - protein, 15 aa, Mycobacterium tuberculosis
ESSQPPVAFSSSIEK

SEQ ID NO: 221 - protein, 21 aa, Mycobacterium tuberculosis
NCQTVLAPCSPNPCENAAVCK

SEQ ID NO: 222 - protein, 14 aa, Mycobacterium tuberculosis
GQDTQFWAGHYGAR

SEQ ID NO: 223 - protein, 15 aa, Mycobacterium tuberculosis
MTENVVCTGAVNAVK

SEQ ID NO: 224 - protein, 15 aa, Mycobacterium tuberculosis
YPSTSEAVNIQGISK

SEQ ID NO: 225 - protein, 15 aa, Mycobacterium tuberculosis
AGFPCFKPSGAAPQR

SEQ ID NO: 226 - protein, 14 aa, Mycobacterium tuberculosis
STLEEQGLHVHSVR

SEQ ID NO: 227 - protein, 16 aa, Mycobacterium tuberculosis
GTANTCIPSISSIGSK

SEQ ID NO: 228 - protein, 13 aa, Mycobacterium tuberculosis
SFNQNSHLIIHQR

SEQ ID NO: 229 - protein, 13 aa, Mycobacterium tuberculosis
IPIDNMTNEMEQR

SEQ ID NO: 230 - protein, 15 aa, Mycobacterium tuberculosis
INLPAPNPDHVGGYK

SEQ ID NO: 231 - protein, 16 aa, Mycobacterium tuberculosis
SPGTHLGALAQTVQGR

SEQ ID NO: 232 - protein, 21 aa, Mycobacterium tuberculosis
LSNCDPPPTYEEATGQVNLQR

SEQ ID NO: 233 - protein, 14 aa, Mycobacterium tuberculosis
EDISACLQGTHGFR

SEQ ID NO: 234 - protein, 13 aa, Mycobacterium tuberculosis
EDCCPGKPLNVFR

SEQ ID NO: 235 - protein, 13 aa, Mycobacterium tuberculosis
ECDMCFSQASSLR

SEQ ID NO: 236 - protein, 14 aa, Mycobacterium tuberculosis
DCAANTFIEDSGYK

SEQ ID NO: 237 - protein, 14 aa, Mycobacterium tuberculosis
LQESGQVTISELCK

SEQ ID NO: 238 - protein, 15 aa, Mycobacterium tuberculosis
SQACGGNLGSIEELR

SEQ ID NO: 239 - protein, 27 aa, Mycobacterium tuberculosis
GPHPQALPGHLPGAGDSGAGAGGGVVR

SEQ ID NO: 240 - protein, 14 aa, Mycobacterium tuberculosis
LEYSGAISAHCNLR

SEQ ID NO: 241 - protein, 15 aa, Mycobacterium tuberculosis
MGVTCVSQMPVAEGK

SEQ ID NO: 242 - protein, 13 aa, Mycobacterium tuberculosis
TCDFFSPYENGEK

SEQ ID NO: 243 - protein, 13 aa, Mycobacterium tuberculosis
FGYYGDALQQDCR

SEQ ID NO: 244 - protein, 14 aa, Mycobacterium tuberculosis
LLSLHSPNSYYGSR

SEQ ID NO: 245 - protein, 15 aa, Mycobacterium tuberculosis
YQVSEEVPSGTVIGK

SEQ ID NO: 246 - protein, 15 aa, Mycobacterium tuberculosis
ESTATLLGCNASIQK

SEQ ID NO: 247 - protein, 14 aa, Mycobacterium tuberculosis
TSVTTSISEPWTQR

SEQ ID NO: 248 - protein, 13 aa, Mycobacterium tuberculosis
MSYSCCLPSLGCR

SEQ ID NO: 249 - protein, 13 aa, Mycobacterium tuberculosis
SEYSSYPDINFNR

SEQ ID NO: 250 - protein, 15 aa, Mycobacterium tuberculosis
HSHGLALQPSFPGSR

SEQ ID NO: 251 - protein, 17 aa, Mycobacterium tuberculosis
LPYSGRPAPAPAAAPGV

SEQ ID NO: 252 - protein, 14 aa, Mycobacterium tuberculosis
AEQDITTLEQSISR

SEQ ID NO: 253 - protein, 15 aa, Mycobacterium tuberculosis
VMSPENFPTASVEGK

SEQ ID NO: 254 - protein, 16 aa, Mycobacterium tuberculosis
GGNQEIGPLPPTGNLK

SEQ ID NO: 255 - protein, 14 aa, Mycobacterium tuberculosis
GAEVTYMNMTAYNK

SEQ ID NO: 256 - protein, 16 aa, Mycobacterium tuberculosis
SPAARPPVPAPPALPR

SEQ ID NO: 257 - protein, 13 aa, Mycobacterium tuberculosis
CLVPGYYSTHLQR

SEQ ID NO: 258 - protein, 15 aa, Mycobacterium tuberculosis
LAPISEEGKPQLVGR

SEQ ID NO: 259 - protein, 13 aa, Mycobacterium tuberculosis
DEDLQEMENLAQR

SEQ ID NO: 260 - protein, 14 aa, Mycobacterium tuberculosis
WIHFGTEVTNSSGR

SEQ ID NO: 261 - protein, 17 aa, Mycobacterium tuberculosis
VLPMVPAPPGSSAAAAR

SEQ ID NO: 262 - protein, 14 aa, Mycobacterium tuberculosis
SNFSPHFASSNQLR

SEQ ID NO: 263 - protein, 11 aa, Mycobacterium tuberculosis
CLHCLYSCHWR

SEQ ID NO: 264 - protein, 14 aa, Mycobacterium tuberculosis
VGNIPYEATEEQLK

SEQ ID NO: 265 - protein, 15 aa, Mycobacterium tuberculosis
HPDEAAFFDTASTGK

SEQ ID NO: 266 - protein, 14 aa, Mycobacterium tuberculosis
SSLTQEEAPVSWEK

SEQ ID NO: 267 - protein, 13 aa, Mycobacterium tuberculosis
VLNQYTDTIIQER

SEQ ID NO: 268 - protein, 14 aa, Mycobacterium tuberculosis
LEQGENVFLQATDK

SEQ ID NO: 269 - protein, 14 aa, Mycobacterium tuberculosis
MAFMAATDHSNQLR

SEQ ID NO: 270 - protein, 23 aa, Mycobacterium tuberculosis
MNSPSQSSPGMNPGQPTSMLSPR

SEQ ID NO: 271 - protein, 13 aa, Mycobacterium tuberculosis
FVYPVPYTTRPPR

SEQ ID NO: 272 - protein, 12 aa, Mycobacterium tuberculosis
WDSNICELHYTR

SEQ ID NO: 273 - protein, 23 aa, Mycobacterium tuberculosis
YPAEEPASAWTPSPPPVTTSSSK

SEQ ID NO: 274 - protein, 15 aa, Mycobacterium tuberculosis
TSANPETLLGEMEAK

SEQ ID NO: 275 - protein, 14 aa, Mycobacterium tuberculosis
MTQPFPTQFAPQAK

SEQ ID NO: 276 - protein, 13 aa, Mycobacterium tuberculosis
TFSQMSSLVYHHR

SEQ ID NO: 277 - protein, 15 aa, Mycobacterium tuberculosis
VGQCVVVFSQAPSGR

SEQ ID NO: 278 - protein, 15 aa, Mycobacterium tuberculosis
NPSTNVSVVVFDSTK

SEQ ID NO: 279 - protein, 13 aa, Mycobacterium tuberculosis
VLMVETHNEIYDK

SEQ ID NO: 280 - protein, 15 aa, Mycobacterium tuberculosis
MDNCLAAAALNGVDR

SEQ ID NO: 281 - protein, 14 aa, Mycobacterium tuberculosis
LVIECGADCNILSK

SEQ ID NO: 282 - protein, 13 aa, Mycobacterium tuberculosis
FCCEDGTTIVNFK

SEQ ID NO: 283 - protein, 14 aa, Mycobacterium tuberculosis
YHYAEISSQVPLGK

SEQ ID NO: 284 - protein, 13 aa, Mycobacterium tuberculosis
LQTLSIQQCLPHR

SEQ ID NO: 285 - protein, 15 aa, Mycobacterium tuberculosis
LVMVSTLDTSSQPGR

SEQ ID NO: 286 - protein, 16 aa, Mycobacterium tuberculosis
QPPPGIVAPAAMLSSR

SEQ ID NO: 287 - protein, 15 aa, Mycobacterium tuberculosis
SPLQAVEPISTSVHK

SEQ ID NO: 288 - protein, 14 aa, Mycobacterium tuberculosis
IVETDESQGIFVEK

SEQ ID NO: 289 - protein, 14 aa, Mycobacterium tuberculosis
LSWPQSTGICSNIK

SEQ ID NO: 290 - protein, 17 aa, Mycobacterium tuberculosis
SVLDLGSGCGATAIAAK

SEQ ID NO: 291 - protein, 14 aa, Mycobacterium tuberculosis
AAGFDEIEQDLTQR

SEQ ID NO: 292 - protein, 14 aa, Mycobacterium tuberculosis
QTVMTSATWPDTVR

SEQ ID NO: 293 - protein, 13 aa, Mycobacterium tuberculosis
TAFTNHQIYELEK

SEQ ID NO: 294 - protein, 17 aa, Mycobacterium tuberculosis
APSSPALQALAGQAGVR

SEQ ID NO: 295 - protein, 16 aa, Mycobacterium tuberculosis
MEPAVGGPGPLIVNNK

SEQ ID NO: 296 - protein, 13 aa, Mycobacterium tuberculosis
QVHILQQNCIALR

SEQ ID NO: 297 - protein, 22 aa, Mycobacterium tuberculosis
VEVSGDASCCSPDPISPEDLPR

SEQ ID NO: 298 - protein, 15 aa, Mycobacterium tuberculosis
EGFPTDAPYPTTLGK

SEQ ID NO: 299 - protein, 16 aa, Mycobacterium tuberculosis
SAYALGGLGSGICPNR

SEQ ID NO: 300 - protein, 14 aa, Mycobacterium tuberculosis
ASSLCHHASLPWVK

SEQ ID NO: 301 - protein, 13 aa, Mycobacterium tuberculosis
FLNDTSLPHSCFR

SEQ ID NO: 302 - protein, 14 aa, Mycobacterium tuberculosis
FLIDSNGQVITTER

SEQ ID NO: 303 - protein, 13 aa, Mycobacterium tuberculosis
IEGENYLPQPIYR

SEQ ID NO: 304 - protein, 13 aa, Mycobacterium tuberculosis
VTGLIENHDYEFR

SEQ ID NO: 305 - protein, 15 aa, Mycobacterium tuberculosis
ILFVSQGSEIASQGR

SEQ ID NO: 306 - protein, 13 aa, Mycobacterium tuberculosis
DLDQVQLHLEEVR

SEQ ID NO: 307 - protein, 13 aa, Mycobacterium tuberculosis
FNDITADVYSEYR

SEQ ID NO: 308 - protein, 15 aa, Mycobacterium tuberculosis
LCVPGIVALQSPPNK

SEQ ID NO: 309 - protein, 22 aa, Mycobacterium tuberculosis
IEETCQVGMKPPVPGGYTLQGK

SEQ ID NO: 310 - protein, 22 aa, Mycobacterium tuberculosis
VWPQATAPEQAPAPARPYQGVR

SEQ ID NO: 311 - protein, 15 aa, Mycobacterium tuberculosis
LGVEMLSESQLSDGK

SEQ ID NO: 312 - protein, 23 aa, Mycobacterium tuberculosis
QNEHHLEGGFSIGSVGPDGQLGR

SEQ ID NO: 313 - protein, 14 aa, Mycobacterium tuberculosis
NFLETDNEGNGILR

SEQ ID NO: 314 - protein, 17 aa, Mycobacterium tuberculosis
GILFVGSGVSGGEEGAR

SEQ ID NO: 315 - protein, 14 aa, Mycobacterium tuberculosis
VTDATETTITISWR

SEQ ID NO: 316 - protein, 12 aa, Mycobacterium tuberculosis
EWDILETEEHYK

SEQ ID NO: 317 - protein, 21 aa, Mycobacterium tuberculosis
QVRPPSCPVPFPETFNGESSR

SEQ ID NO: 318 - protein, 13 aa, Mycobacterium tuberculosis
VVFHLHESFPRPK

SEQ ID NO: 319 - protein, 14 aa, Mycobacterium tuberculosis
MYQGHMQVVGVTLK

SEQ ID NO: 320 - protein, 23 aa, Mycobacterium tuberculosis
SPGDGGPHDVFTSLPSDCQLGSR

SEQ ID NO: 321 - protein, 15 aa, Mycobacterium tuberculosis
WAAGAMAAPEPLRPR

SEQ ID NO: 322 - protein, 13 aa, Mycobacterium tuberculosis
AQLEEEIIAYEER

SEQ ID NO: 323 - protein, 14 aa, Mycobacterium tuberculosis
MSEDSSALPWSINR

SEQ ID NO: 324 - protein, 13 aa, Mycobacterium tuberculosis
LMNEDPMYSMYAK

SEQ ID NO: 325 - protein, 15 aa, Mycobacterium tuberculosis
GLGAFVIDSDHLGHR

SEQ ID NO: 326 - protein, 15 aa, Mycobacterium tuberculosis
ADANTAAIQAVLYNR

SEQ ID NO: 327 - protein, 14 aa, Mycobacterium tuberculosis
EDIVTEQIDFSAAR

SEQ ID NO: 328 - protein, 20 aa, Mycobacterium tuberculosis
MVRPQDTVAYEDLSEDYTQK

SEQ ID NO: 329 - protein, 12 aa, Mycobacterium tuberculosis
DYFQHPHFSTWK

SEQ ID NO: 330 - protein, 14 aa, Mycobacterium tuberculosis
IVESMQSTLDAEIR

SEQ ID NO: 331 - protein, 22 aa, Mycobacterium tuberculosis
SLSNENYGVYNCSIINEAGAGR

SEQ ID NO: 332 - protein, 13 aa, Mycobacterium tuberculosis
QSWNPFPDFTPQK

SEQ ID NO: 333 - protein, 23 aa, Mycobacterium tuberculosis
LTWPTDAGPDDAAVDTSSEITTK

SEQ ID NO: 334 - protein, 15 aa, Human immunodeficiency virus 1
VHPVHAGPIAPGQMR

SEQ ID NO: 335 - protein, 12 aa, Human immunodeficiency virus 1
PEPTAPPEESFR

SEQ ID NO: 336 - protein, 11 aa, Human immunodeficiency virus 1
SGVETTTPPQK

SEQ ID NO: 337 - protein, 9 aa, Human immunodeficiency virus 1
GSDIAGTTS

SEQ ID NO: 338 - protein, 14 aa, Human immunodeficiency virus 1
QGPKEPFRDYVDRF

SEQ ID NO: 339 - protein, 11 aa, Human immunodeficiency virus 1
  VARIANT 5: note = Thr or Val
  VARIANT 11: note = Lys or Arg
MYSPXSILDIX

SEQ ID NO: 340 - protein, 11 aa, Human immunodeficiency virus 1
  VARIANT 11: note = Lys or Arg
MYSPSSILDIX

SEQ ID NO: 341 - protein, 11 aa, Human immunodeficiency virus 1
TINEEAAEWDR

SEQ ID NO: 342 - protein, 13 aa, Human immunodeficiency virus 1
DTINEEAFAEWDR

SEQ ID NO: 343 - protein, 12 aa, Human immunodeficiency virus 1
  VARIANT 1: note = Glu or Asp
XTINEEAAEWDR

SEQ ID NO: 344 - protein, 11 aa, Human immunodeficiency virus 1
TINEEAADWDR

SEQ ID NO: 345 - protein, 12 aa, Human immunodeficiency virus 1
ETINEEAADWDR

SEQ ID NO: 346 - protein, 12 aa, Human immunodeficiency virus 1
DTINEEAADWDR

SEQ ID NO: 347 - protein, 12 aa, Human immunodeficiency virus 1
  VARIANT 1: note = Glu or Asp
XTINEEAADWDR

SEQ ID NO: 348 - protein, 12 aa, Human immunodeficiency virus 1
DTINEEAAEWER

SEQ ID NO: 349 - protein, 17 aa, Human immunodeficiency virus 1
MQMLKDTINEEAADWDR

SEQ ID NO: 350 - protein, 20 aa, Human immunodeficiency virus 1
VHPVHAGPIPPGQMREPRGS

SEQ ID NO: 351 - protein, 21 aa, Human immunodeficiency virus 1
NKIVRMYSPVSILDIRQGPKE

SEQ ID NO: 352 - protein, 17 aa, Human immunodeficiency virus 1
MQMLKDTINEEAAEWDR

SEQ ID NO: 353 - protein, 20 aa, Human immunodeficiency virus 1
LHPVHAGPIPPGQMREPRGS

SEQ ID NO: 354 - protein, 21 aa, Human immunodeficiency virus 1
NKIVRMYSPVSILDIRQGPKE

SEQ ID NO: 355 - protein, 17 aa, Human immunodeficiency virus 1
MQMLKETINEEAAEWDR

SEQ ID NO: 356 - protein, 20 aa, Human immunodeficiency virus 1
LHPVQAGPVAPGQMREPRGS

SEQ ID NO: 357 - protein, 21 aa, Human immunodeficiency virus 1
NKIVRMYSPSSILDIKQGPKE

SEQ ID NO: 358 - protein, 17 aa, Human immunodeficiency virus 1
MQMLKDTINEEAAEWDR

SEQ ID NO: 359 - protein, 20 aa, Human immunodeficiency virus 1
LHPVHAGPVAPGQMREPRGS

SEQ ID NO: 360 - protein, 21 aa, Human immunodeficiency virus 1
NKIVRMYSPVSILDIKQGPKE

SEQ ID NO: 361 - protein, 17 aa, Human immunodeficiency virus 1
MQMLKETINEEAAEWDR

SEQ ID NO: 362 - protein, 20 aa, Human immunodeficiency virus 1
VHPVHAGPIAPGQMREPRGS

SEQ ID NO: 363 - protein, 21 aa, Human immunodeficiency virus 1
NKIVRMYSPTSILDIRQGPKE

SEQ ID NO: 364 - protein, 17 aa, Human immunodeficiency virus 1
MQMLKETINEEAAEWDR

SEQ ID NO: 365 - protein, 20 aa, Human immunodeficiency virus 1
VHPVHAGPIAPGQMREPRGS

SEQ ID NO: 366 - protein, 21 aa, Human immunodeficiency virus 1
NKIVRMYSPTSILDIRQGPKE

SEQ ID NO: 367 - protein, 17 aa, Human immunodeficiency virus 1
MQMLKETINEEAAEWDR

SEQ ID NO: 368 - protein, 20 aa, Human immunodeficiency virus 1
VHPVHAGPIAPGQMREPRGS

SEQ ID NO: 369 - protein, 21 aa, Human immunodeficiency virus 1
NKIVRMYSPTSILDIRQGPKE

SEQ ID NO: 370 - protein, 17 aa, Human immunodeficiency virus 1
MQMLKETINEEAAEWDR

SEQ ID NO: 371 - protein, 20 aa, Human immunodeficiency virus 1
VHPVHAGPIAPGQMREPRGS

SEQ ID NO: 372 - protein, 21 aa, Human immunodeficiency virus 1
NKIVRMYSPTSILDIRQGPKE

SEQ ID NO: 373 - protein, 17 aa, Human immunodeficiency virus 1
MQMLKETINEEAAEWDR

SEQ ID NO: 374 - protein, 20 aa, Human immunodeficiency virus 1
LHPVHAGPIAPGQMREPRGS

SEQ ID NO: 375 - protein, 21 aa, Human immunodeficiency virus 1
NKIVRMYSPISILDIRQGPKE

SEQ ID NO: 376 - protein, 17 aa, Human immunodeficiency virus 1
MQMLKDTINEEAAEWDR

SEQ ID NO: 377 - protein, 20 aa, Human immunodeficiency virus 1
LHPVHAGPVAPGQMRDPRGS

SEQ ID NO: 378 - protein, 21 aa, Human immunodeficiency virus 1
NKIVRMYSPVSILDIKQGPKE

SEQ ID NO: 379 - protein, 17 aa, Human immunodeficiency virus 1
MQMLKETINEEAAEWDR

SEQ ID NO: 380 - protein, 20 aa, Human immunodeficiency virus 1
VHPVHAGPIAPGQMREPRGS

SEQ ID NO: 381 - protein, 21 aa, Human immunodeficiency virus 1
NKIVRMYSPTSILDIRQGPKE

SEQ ID NO: 382 - protein, 17 aa, Human immunodeficiency virus 1
MQMLKETINEEAAEWDR

SEQ ID NO: 383 - protein, 20 aa, Human immunodeficiency virus 1
LHPAQAGPIAPGQMREPRGS

SEQ ID NO: 384 - protein, 21 aa, Human immunodeficiency virus 1
NKIVRMYSPSSILDIRQGPKE

SEQ ID NO: 385 - protein, 17 aa, Human immunodeficiency virus 1
MQMLKETINEEAAEWDR

SEQ ID NO: 386 - protein, 20 aa, Human immunodeficiency virus 1
LHPVHAGPIAPGQMREPRGS

SEQ ID NO: 387 - protein, 21 aa, Human immunodeficiency virus 1
NKIVRMYSPVSILDIRQGPKE

SEQ ID NO: 388 - protein, 17 aa, Human immunodeficiency virus 1
MQMLKETINEEAAEWDR

SEQ ID NO: 389 - protein, 20 aa, Human immunodeficiency virus 1
VHPVHAGPIAPGQMREPRGS

SEQ ID NO: 390 - protein, 21 aa, Human immunodeficiency virus 1
NKIVRMYSPTSILDIRQGPKE

SEQ ID NO: 391 - protein, 17 aa, Human immunodeficiency virus 1
MQMLKETINEEAAEWDR

SEQ ID NO: 392 - protein, 20 aa, Human immunodeficiency virus 1
LHPAHAGPIAPGQMREPRGS

SEQ ID NO: 393 - protein, 21 aa, Human immunodeficiency virus 1
NKIVRMYSPSSILDIRQGPKE

SEQ ID NO: 394 - protein, 17 aa, Human immunodeficiency virus 1
MQMLKETINEEAAEWDR

SEQ ID NO: 395 - protein, 20 aa, Human immunodeficiency virus 1
LHPVHAGPIAPGQMREPRGS

SEQ ID NO: 396 - protein, 21 aa, Human immunodeficiency virus 1
NKIVRMYSPTSILDIRQGPKE

SEQ ID NO: 397 - protein, 17 aa, Human immunodeficiency virus 1
MQMLKETINEEAAEWDR

SEQ ID NO: 398 - protein, 20 aa, Human immunodeficiency virus 1
LHPVHAGPIAPGQMREPRGS

SEQ ID NO: 399 - protein, 21 aa, Human immunodeficiency virus 1
NKIVRMYSPTSILDIRQGPKE

SEQ ID NO: 400 - protein, 17 aa, Human immunodeficiency virus 1
MQMLKETINEEAAEWDR

SEQ ID NO: 401 - protein, 20 aa, Human immunodeficiency virus 1
LHPVHAGPIAPGQMREPRGS

SEQ ID NO: 402 - protein, 21 aa, Human immunodeficiency virus 1
NKIVRMYSPISILDIRQGPKE

SEQ ID NO: 403 - protein, 17 aa, Human immunodeficiency virus 1
MQMLKDTINEEAAEWDR

SEQ ID NO: 404 - protein, 20 aa, Human immunodeficiency virus 1
LHPVHAGPIPPGQMREPRGS

SEQ ID NO: 405 - protein, 21 aa, Human immunodeficiency virus 1
NKIVRMYSPVSILDIRQGPKE

SEQ ID NO: 406 - protein, 17 aa, Human immunodeficiency virus 1
MQMLKETINEEAAEWDR

SEQ ID NO: 407 - protein, 20 aa, Human immunodeficiency virus 1
LHPVHAGPIAPGQMREPRGS

SEQ ID NO: 408 - protein, 21 aa, Human immunodeficiency virus 1
NKIVRMYSPTSILDIRQGPKE

SEQ ID NO: 409 - protein, 17 aa, Human immunodeficiency virus 1
MQMLKETINEEAAEWDR

SEQ ID NO: 410 - protein, 20 aa, Human immunodeficiency virus 1
LHPVHAGPIAPGQMREPRGS

SEQ ID NO: 411 - protein, 21 aa, Human immunodeficiency virus 1
NKIVRMYSPTSILDIRQGPKE

SEQ ID NO: 412 - protein, 17 aa, Human immunodeficiency virus 1
MQMLKDTINEEAAEWDR

SEQ ID NO: 413 - protein, 20 aa, Human immunodeficiency virus 1
LHPVHAGPVAPGQMREPRGS

SEQ ID NO: 414 - protein, 21 aa, Human immunodeficiency virus 1
NKIVRMYSPVSILDIKQGPKE

SEQ ID NO: 415 - protein, 17 aa, Human immunodeficiency virus 1
MQMLKDTINEEAAEWDR

SEQ ID NO: 416 - protein, 20 aa, Human immunodeficiency virus 1
LHPVHAGPVAPGQMRDPRGS

SEQ ID NO: 417 - protein, 21 aa, Human immunodeficiency virus 1
NKIVRMYSPVSILDIKQGPKE

SEQ ID NO: 418 - protein, 17 aa, Human immunodeficiency virus 1
MQMLKETINEEAAEWDR

SEQ ID NO: 419 - protein, 20 aa, Human immunodeficiency virus 1
LHPVHAGPIAPGQMREPRGS

SEQ ID NO: 420 - protein, 21 aa, Human immunodeficiency virus 1
NKIVRMYSPVSILDIRQGPKE

SEQ ID NO: 421 - protein, 17 aa, Human immunodeficiency virus 1
MQMLKETINDEAAEWDR

SEQ ID NO: 422 - protein, 20 aa, Human immunodeficiency virus 1
LHPVHAGPVAPGQMREPRGS

SEQ ID NO: 423 - protein, 21 aa, Human immunodeficiency virus 1
NKIVRMYSPVSILDIRQGPKE

SEQ ID NO: 424 - protein, 17 aa, Human immunodeficiency virus 1
MQMLKETINEEAAEWDR

SEQ ID NO: 425 - protein, 20 aa, Human immunodeficiency virus 1
LHPVHAGPIAPGQMREPRGS

SEQ ID NO: 426 - protein, 21 aa, Human immunodeficiency virus 1
NKIVRMYSPVSILDIRQGPKE

SEQ ID NO: 427 - protein, 17 aa, Human immunodeficiency virus 1
MQMLKDTINEEAAEWDR

SEQ ID NO: 428 - protein, 20 aa, Human immunodeficiency virus 1
LHPTQAGPIPPGQIREPRGS

SEQ ID NO: 429 - protein, 21 aa, Human immunodeficiency virus 1
NKIVRMYSPVGILDIRQGPKE

SEQ ID NO: 430 - protein, 17 aa, Human immunodeficiency virus 1
MQMLKDTINEEAAEWDR

SEQ ID NO: 431 - protein, 20 aa, Human immunodeficiency virus 1
LHPVHAGPAPPGQMREPRGS

SEQ ID NO: 432 - protein, 21 aa, Human immunodeficiency virus 1
NKIVRMYSPVSILDIKQGPKE

SEQ ID NO: 433 - protein, 17 aa, Human immunodeficiency virus 1
MQMLKDTINEEAAEWDR

SEQ ID NO: 434 - protein, 20 aa, Human immunodeficiency virus 1
LHPVHAGPIPPGQMREPRGS

SEQ ID NO: 435 - protein, 21 aa, Human immunodeficiency virus 1
NKIVRMYSPVSILDIKQGPKE

SEQ ID NO: 436 - protein, 17 aa, Human immunodeficiency virus 1
MQMLKDTINEEAAEWDR

SEQ ID NO: 437 - protein, 20 aa, Human immunodeficiency virus 1
LHPVHAGPIPPGQMREPRGS

SEQ ID NO: 438 - protein, 21 aa, Human immunodeficiency virus 1
NKIVRMYSPVSILDIKQGPKE

SEQ ID NO: 439 - protein, 17 aa, Human immunodeficiency virus 1
MQMLKDTINDEAAEWDR

SEQ ID NO: 440 - protein, 20 aa, Human immunodeficiency virus 1
IHPQQAGPIPPGQIREPSGS

SEQ ID NO: 441 - protein, 21 aa, Human immunodeficiency virus 1
NKIVRMYSPVSILDIRQGPKE

SEQ ID NO: 442 - protein, 17 aa, Human immunodeficiency virus 1
MQMLKDTINEEAAEWDR

SEQ ID NO: 443 - protein, 20 aa, Human immunodeficiency virus 1
MHPQQAGPFPPGQIREPRGS

SEQ ID NO: 444 - protein, 21 aa, Human immunodeficiency virus 1
NKIVRMYSPVSILDIRQGPKE

SEQ ID NO: 445 - protein, 17 aa, Human immunodeficiency virus 1
MQMLKDTINEEAAEWDR

SEQ ID NO: 446 - protein, 20 aa, Human immunodeficiency virus 1
VHPVHAGPIPPGQMREPRGS

SEQ ID NO: 447 - protein, 21 aa, Human immunodeficiency virus 1
NKIVRMYSPVSILDIKQGPKE

SEQ ID NO: 448 - protein, 17 aa, Human immunodeficiency virus 1
MQMLKDTINEEAAEWDR

SEQ ID NO: 449 - protein, 20 aa, Human immunodeficiency virus 1
LHPVHAGPIPPGQMREPRGS

SEQ ID NO: 450 - protein, 21 aa, Human immunodeficiency virus 1
NKIVRMYSPVSILDIKQGPKE

SEQ ID NO: 451 - protein, 17 aa, Human immunodeficiency virus 1
MQMLKDTINEEAAEWDR

SEQ ID NO: 452 - protein, 20 aa, Human immunodeficiency virus 1
VHPVHAGPVAPGQVREPRGS

SEQ ID NO: 453 - protein, 21 aa, Human immunodeficiency virus 1
NKIVRMYSPVSILDIRQGPKE

SEQ ID NO: 454 - protein, 17 aa, Human immunodeficiency virus 1
MQMLKDTINEEAAEWDR

SEQ ID NO: 455 - protein, 20 aa, Human immunodeficiency virus 1
VHPVHAGPIAPGQVREPRGS

SEQ ID NO: 456 - protein, 21 aa, Human immunodeficiency virus 1
NKIVRMYSPVSILDIRQGPKE

SEQ ID NO: 457 - protein, 17 aa, Human immunodeficiency virus 1
MQMLKDTINDEAAEWDR

SEQ ID NO: 458 - protein, 20 aa, Human immunodeficiency virus 1
LHPVHAGPIPPGQMREPRGS

SEQ ID NO: 459 - protein, 21 aa, Human immunodeficiency virus 1
NKIVRMYSPVSILDIRQGPKE

SEQ ID NO: 460 - protein, 17 aa, Human immunodeficiency virus 1
MQMLKDTINEEAAEWDR

SEQ ID NO: 461 - protein, 20 aa, Human immunodeficiency virus 1
MHPVQAGPIPPGQIREPRGS

SEQ ID NO: 462 - protein, 21 aa, Human immunodeficiency virus 1
NKIVRMYSPVSILDIRQGPKE

SEQ ID NO: 463 - protein, 17 aa, Human immunodeficiency virus 1
MQMLKEVINEEAAEWDR

SEQ ID NO: 464 - protein, 20 aa, Human immunodeficiency virus 1
THPAPVGPLPPGQMRDPRGS

SEQ ID NO: 465 - protein, 21 aa, Human immunodeficiency virus 1
NRIVRMYSPVSILEIKQGPKE

SEQ ID NO: 466 - protein, 17 aa, Human immunodeficiency virus 1
MQMLKEVINEEAADWDR

SEQ ID NO: 467 - protein, 20 aa, Human immunodeficiency virus 1
THPVPVGPLPPGQLRDPRGS

SEQ ID NO: 468 - protein, 21 aa, Human immunodeficiency virus 1
NRIVRMYSPVSILEIKQGPKE

SEQ ID NO: 469 - protein, 17 aa, Human immunodeficiency virus 1
LQVLKEVINEEAVEWDR

SEQ ID NO: 470 - protein, 20 aa, Human immunodeficiency virus 1
THPPPVGPLPPGQIREPTGS

SEQ ID NO: 471 - protein, 21 aa, Human immunodeficiency virus 1
NKMVKMYSPVSILDIKQGPKE

SEQ ID NO: 472 - protein, 17 aa, Human immunodeficiency virus 1
LQVLKEVINEEAAEWDR

SEQ ID NO: 473 - protein, 20 aa, Human immunodeficiency virus 1
THPPAMGPLPPGQIREPTGS

SEQ ID NO: 474 - protein, 21 aa, Human immunodeficiency virus 1
NKMVKMYSPVSILDIRQGPKE

SEQ ID NO: 475 - protein, 17 aa, Human immunodeficiency virus 2
MQIIREIINEEAADWDA

SEQ ID NO: 476 - protein, 19 aa, Human immunodeficiency virus 2
NHPIPGPLPAGQLRDPRGS

SEQ ID NO: 477 - protein, 21 aa, Human immunodeficiency virus 2
QKCVRMYNPTNILDIKQGPKE

SEQ ID NO: 478 - protein, 17 aa, Human immunodeficiency virus 2
MQIIREIINEEAAEWDV

SEQ ID NO: 479 - protein, 19 aa, Human immunodeficiency virus 2
QHPIPGPLPAGQLREPRGS

SEQ ID NO: 480 - protein, 21 aa, Human immunodeficiency virus 2
QKCVRMYNPTNILDIKQGPKE

SEQ ID NO: 481 - protein, 17 aa, Human immunodeficiency virus 2
MQIIREIINEEAADWDV

SEQ ID NO: 482 - protein, 19 aa, Human immunodeficiency virus 2
QHPIPGPLPAGQLREPRGS

SEQ ID NO: 483 - protein, 21 aa, Human immunodeficiency virus 2
QKCVRMYNPTNILDVKQGPKE

SEQ ID NO: 484 - protein, 17 aa, Human immunodeficiency virus 2
MQIIREIINEEAADWDA

SEQ ID NO: 485 - protein, 19 aa, Human immunodeficiency virus 2
QHPIPGPLPAGQLREPRGS

SEQ ID NO: 486 - protein, 21 aa, Human immunodeficiency virus 2
QKCVRMYNPTNILDIKQGPKE

SEQ ID NO: 487 - protein, 17 aa, Human immunodeficiency virus 2
MQIIREIINEEAADWDA

SEQ ID NO: 488 - protein, 19 aa, Human immunodeficiency virus 2
QHPIPGPLPAGQLRDPRGS

SEQ ID NO: 489 - protein, 21 aa, Human immunodeficiency virus 2
QKCVRMYNPTNILDVKQGPKE

SEQ ID NO: 490 - protein, 17 aa, Human immunodeficiency virus 2
MQIIREIINEEAADWDS

SEQ ID NO: 491 - protein, 19 aa, Human immunodeficiency virus 2
QHPIPGPLPAGQLRDPRGS

SEQ ID NO: 492 - protein, 21 aa, Human immunodeficiency virus 2
QKCVRKYNPTNILDIKQGPKE

SEQ ID NO: 493 - protein, 17 aa, Human immunodeficiency virus 2
MQIIREIINDEAADWDA

SEQ ID NO: 494 - protein, 19 aa, Human immunodeficiency virus 2
QHPIPGPLPAGQLRDPRGS

SEQ ID NO: 495 - protein, 21 aa, Human immunodeficiency virus 2
QKCVRMYNPTNILDVKQGPKE

SEQ ID NO: 496 - protein, 17 aa, Human immunodeficiency virus 2
MQIIREIINEEAADWDV

SEQ ID NO: 497 - protein, 19 aa, Human immunodeficiency virus 2
AHPIPGPLPAGQLREPRGS

SEQ ID NO: 498 - protein, 21 aa, Human immunodeficiency virus 2
QKCVRMYNPTNILDINQGPKE

SEQ ID NO: 499 - protein, 17 aa, Human immunodeficiency virus 2
MQIIREIINEEAADWDV

SEQ ID NO: 500 - protein, 19 aa, Human immunodeficiency virus 2
QHPIPGPLPAGQLRDPRGS

SEQ ID NO: 501 - protein, 21 aa, Human immunodeficiency virus 2
QKCVRMYNPTNILDIKQGPKE

SEQ ID NO: 502 - protein, 17 aa, Human immunodeficiency virus 2
MQIIREIINEEAADWDQ

SEQ ID NO: 503 - protein, 19 aa, Human immunodeficiency virus 2
QHPIPGPLPAGQLRDPRGS

SEQ ID NO: 504 - protein, 21 aa, Human immunodeficiency virus 2
QKCVRMYNPTNILDIKQGPKE

SEQ ID NO: 505 - protein, 17 aa, Human immunodeficiency virus 2
MQIIREIINEEAADWDQ

SEQ ID NO: 506 - protein, 19 aa, Human immunodeficiency virus 2
QHPSPGPMPAGQLRDPRGS

SEQ ID NO: 507 - protein, 21 aa, Human immunodeficiency virus 2
QKCVRMYNPTNILDIKQGPKE

SEQ ID NO: 508 - protein, 17 aa, Human immunodeficiency virus 2
MQIIREIINEEAADWDQ

SEQ ID NO: 509 - protein, 19 aa, Human immunodeficiency virus 2
QHPSPGPMPAGQLREPRGS

SEQ ID NO: 510 - protein, 21 aa, Human immunodeficiency virus 2
QKCVRMYNPTNILDIKQGPKE

SEQ ID NO: 511 - protein, 17 aa, Simian immunodeficiency virus
MQIIREIINEEAADWDL

SEQ ID NO: 512 - protein, 19 aa, Simian immunodeficiency virus
QHPQPGPLPAGQLREPRGS

SEQ ID NO: 513 - protein, 21 aa, Simian immunodeficiency virus
QKCVRMYNPTNILDVKQGPKE

SEQ ID NO: 514 - protein, 17 aa, Simian immunodeficiency virus
MQIIREIINEEAADWDL

SEQ ID NO: 515 - protein, 19 aa, Simian immunodeficiency virus
QHPQPGPIPPGQLREPRGS

SEQ ID NO: 516 - protein, 21 aa, Simian immunodeficiency virus
QKCVRMYNPTNILDVKQGPKE

SEQ ID NO: 517 - protein, 17 aa, Simian immunodeficiency virus
MQIIRDIINEEAADWDL

SEQ ID NO: 518 - protein, 18 aa, Simian immunodeficiency virus
QHPQPAPQQGQLREPSGS

SEQ ID NO: 519 - protein, 21 aa, Simian immunodeficiency virus
QKCVRMYNPTNILDVKQGPKE

SEQ ID NO: 520 - protein, 17 aa, Simian immunodeficiency virus
MQIIRDIINEEAADWDL

SEQ ID NO: 521 - protein, 18 aa, Simian immunodeficiency virus
QHPQPAPQQGQLREPSGS

SEQ ID NO: 522 - protein, 21 aa, Simian immunodeficiency virus
QKCVRMYNPTNILDVKQGPKE

SEQ ID NO: 523 - protein, 17 aa, Simian immunodeficiency virus
MQIIRDIINEEAADWDL

SEQ ID NO: 524 - protein, 18 aa, Simian immunodeficiency virus
QHPQPAPQQGQLREPSGS

SEQ ID NO: 525 - protein, 21 aa, Simian immunodeficiency virus
QKCVRMYNPTNILDVKQGPKE

SEQ ID NO: 526 - protein, 17 aa, Simian immunodeficiency virus
MQIIRDIINEEAADWDL

SEQ ID NO: 527 - protein, 18 aa, Simian immunodeficiency virus
QHPQPAPQQGQLREPSGS

SEQ ID NO: 528 - protein, 21 aa, Simian immunodeficiency virus
QKCVRMYNPTNILDVKQGPKE

SEQ ID NO: 529 - protein, 17 aa, Simian immunodeficiency virus
MQIIRDIINEEAADWDL

SEQ ID NO: 530 - protein, 18 aa, Simian immunodeficiency virus
QHPQQAPQQGQLREPSGS

SEQ ID NO: 531 - protein, 21 aa, Simian immunodeficiency virus
QKCVRMYNPTNILDVKQGPKE

SEQ ID NO: 532 - protein, 17 aa, Simian immunodeficiency virus
MQIIRDIINEEAADWDL

SEQ ID NO: 533 - protein, 18 aa, Simian immunodeficiency virus
QHPQPAPQQGQLREPSGS

SEQ ID NO: 534 - protein, 21 aa, Simian immunodeficiency virus
QKCVRMYNPTNILDVKQGPKE

SEQ ID NO: 535 - protein, 17 aa, Simian immunodeficiency virus
MQIIRDIINEEAADWDL

SEQ ID NO: 536 - protein, 18 aa, Simian immunodeficiency virus
QHPQPAPQQGQLREPSGS

SEQ ID NO: 537 - protein, 21 aa, Simian immunodeficiency virus
RLQKCVYNPINILDVKQRPKE

SEQ ID NO: 538 - protein, 17 aa, Simian immunodeficiency virus
MQIIREIINEEAADWDV

SEQ ID NO: 539 - protein, 19 aa, Simian immunodeficiency virus
QHPQPGPLPAGQLREPSGS

SEQ ID NO: 540 - protein, 21 aa, Simian immunodeficiency virus
QKCVRMYNPVNILDIKQGPKE

SEQ ID NO: 541 - protein, 37 aa, Human immunodeficiency virus 1
MQMLKDTINEEAADWDRVHPVHAGPIPPGQMREPRGS

SEQ ID NO: 542 - protein, 37 aa, Human immunodeficiency virus 1
MQMLKDTINEEAAEWDRLHPVHAGPIPPGQMREPRGS

SEQ ID NO: 543 - protein, 37 aa, Human immunodeficiency virus 1
MQMLKETINEEAAEWDRLHPVQAGPVAPGQMREPRGS

SEQ ID NO: 544 - protein, 37 aa, Human immunodeficiency virus 1
MQMLKDTINEEAAEWDRLHPVHAGPVAPGQMREPRGS

SEQ ID NO: 545 - protein, 37 aa, Human immunodeficiency virus 1
MQMLKETINEEAAEWDRVHPVHAGPIAPGQMREPRGS

SEQ ID NO: 546 - protein, 37 aa, Human immunodeficiency virus 1; identical to SEQ ID NO: 545

SEQ ID NO: 547 - protein, 37 aa, Human immunodeficiency virus 1; identical to SEQ ID NO: 545

SEQ ID NO: 548 - protein, 37 aa, Human immunodeficiency virus 1; identical to SEQ ID NO: 545

SEQ ID NO: 549 - protein, 37 aa, Human immunodeficiency virus 1
MQMLKETINEEAAEWDRLHPVHAGPIAPGQMREPRGS

SEQ ID NO: 550 - protein, 37 aa, Human immunodeficiency virus 1
MQMLKDTINEEAAEWDRLHPVHAGPVAPGQMRDPRGS

SEQ ID NO: 551 - protein, 37 aa, Human immunodeficiency virus 1; identical to SEQ ID NO: 545

SEQ ID NO: 552 - protein, 37 aa, Human immunodeficiency virus 1
MQMLKETINEEAAEWDRLHPAQAGPIAPGQMREPRGS

SEQ ID NO: 553 - protein, 37 aa, Human immunodeficiency virus 1; identical to SEQ ID NO: 549

SEQ ID NO: 554 - protein, 37 aa, Human immunodeficiency virus 1; identical to SEQ ID NO: 545

SEQ ID NO: 555 - protein, 37 aa, Human immunodeficiency virus 1
MQMLKETINEEAAEWDRLHPAHAGPIAPGQMREPRGS

SEQ ID NO: 556 - protein, 37 aa, Human immunodeficiency virus 1; identical to SEQ ID NO: 549

SEQ ID NO: 557 - protein, 37 aa, Human immunodeficiency virus 1; identical to SEQ ID NO: 549

SEQ ID NO: 558 - protein, 37 aa, Human immunodeficiency virus 1; identical to SEQ ID NO: 549

SEQ ID NO: 559 - protein, 37 aa, Human immunodeficiency virus 1; identical to SEQ ID NO: 542

SEQ ID NO: 560 - protein, 37 aa, Human immunodeficiency virus 1; identical to SEQ ID NO: 549

SEQ ID NO: 561 - protein, 37 aa, Human immunodeficiency virus 1; identical to SEQ ID NO: 549

SEQ ID NO: 562 - protein, 37 aa, Human immunodeficiency virus 1; identical to SEQ ID NO: 544

SEQ ID NO: 563 - protein, 37 aa, Human immunodeficiency virus 1; identical to SEQ ID NO: 550

SEQ ID NO: 564 - protein, 37 aa, Human immunodeficiency virus 1; identical to SEQ ID NO: 549

SEQ ID NO: 565 - protein, 37 aa, Human immunodeficiency virus 1
MQMLKETINDEAAEWDRLHPVHAGPVAPGQMREPRGS

SEQ ID NO: 566 - protein, 37 aa, Human immunodeficiency virus 1; identical to SEQ ID NO: 549

SEQ ID NO: 567 - protein, 37 aa, Human immunodeficiency virus 1
MQMLKDTINEEAAEWDRLHPTQAGPIPPGQIREPRGS

SEQ ID NO: 568 - protein, 37 aa, Human immunodeficiency virus 1
MQMLKDTINEEAAEWDRLHPVHAGPAPPGQMREPRGS

SEQ ID NO: 569 - protein, 37 aa, Human immunodeficiency virus 1; identical to SEQ ID NO: 542

SEQ ID NO: 570 - protein, 37 aa, Human immunodeficiency virus 1; identical to SEQ ID NO: 542

SEQ ID NO: 571 - protein, 37 aa, Human immunodeficiency virus 1
MQMLKDTINDEAAEWDRIHPQQAGPIPPGQIREPSGS

SEQ ID NO: 572 - protein, 37 aa, Human immunodeficiency virus 1
MQMLKDTINEEAAEWDRMHPQQAGPFPPGQIREPRGS

SEQ ID NO: 573 - protein, 37 aa, Human immunodeficiency virus 1
MQMLKDTINEEAAEWDRVHPVHAGPIPPGQMREPRGS

SEQ ID NO: 574 - protein, 37 aa, Human immunodeficiency virus 1; identical to SEQ ID NO: 542

SEQ ID NO: 575 - protein, 37 aa, Human immunodeficiency virus 1
MQMLKDTINEEAAEWDRVHPVHAGPVAPGQVREPRGS

SEQ ID NO: 576 - protein, 37 aa, Human immunodeficiency virus 1
MQMLKDTINEEAAEWDRVHPVHAGPIAPGQVREPRGS

SEQ ID NO: 577 - protein, 37 aa, Human immunodeficiency virus 1
MQMLKDTINDEAAEWDRLHPVHAGPIPPGQMREPRGS

SEQ ID NO: 578 - protein, 37 aa, Human immunodeficiency virus 1
MQMLKDTINEEAAEWDRMHPVQAGPIPPGQIREPRGS

SEQ ID NO: 579 - protein, 37 aa, Human immunodeficiency virus 1
MQMLKEVINEEAAEWDRTHPAPVGPLPPGQMRDPRGS

SEQ ID NO: 580 - protein, 37 aa, Human immunodeficiency virus 1
MQMLKEVINEEAADWDRTHPVPVGPLPPGQLRDPRGS

SEQ ID NO: 581 - protein, 37 aa, Human immunodeficiency virus 1
LQVLKEVINEEAVEWDRTHPPPVGPLPPGQIREPTGS

SEQ ID NO: 582 - protein, 37 aa, Human immunodeficiency virus 1
LQVLKEVINEEAAEWDRTHPPAMGPLPPGQIREPTGS

SEQ ID NO: 583 - protein, 36 aa, Human immunodeficiency virus 2
MQIIREIINEEAADWDANHPIPGPLPAGQLRDPRGS

SEQ ID NO: 584 - protein, 36 aa, Human immunodeficiency virus 2
MQIIREIINEEAAEWDVQHPIPGPLPAGQLREPRGS

SEQ ID NO: 585 - protein, 36 aa, Human immunodeficiency virus 2
MQIIREIINEEAADWDVQHPIPGPLPAGQLREPRGS

SEQ ID NO: 586 - protein, 36 aa, Human immunodeficiency virus 2
MQIIREIINEEAADWDAQHPIPGPLPAGQLREPRGS

SEQ ID NO: 587 - protein, 36 aa, Human immunodeficiency virus 2
MQIIREIINEEAADWDAQHPIPGPLPAGQLRDPRGS

SEQ ID NO: 588 - protein, 36 aa, Human immunodeficiency virus 2
MQIIREIINEEAADWDSQHPIPGPLPAGQLRDPRGS

SEQ ID NO: 589 - protein, 36 aa, Human immunodeficiency virus 2
MQIIREIINDEAADWDAQHPIPGPLPAGQLRDPRGS

SEQ ID NO: 590 - protein, 36 aa, Human immunodeficiency virus 2
MQIIREIINEEAADWDVAHPIPGPLPAGQLREPRGS

SEQ ID NO: 591 - protein, 36 aa, Human immunodeficiency virus 2
MQIIREIINEEAADWDVQHPIPGPLPAGQLRDPRGS

SEQ ID NO: 592 - protein, 36 aa, Human immunodeficiency virus 2
MQIIREIINEEAADWDQQHPIPGPLPAGQLRDPRGS

SEQ ID NO: 593 - protein, 36 aa, Human immunodeficiency virus 2
MQIIREIINEEAADWDQQHPSPGPMPAGQLRDPRGS

SEQ ID NO: 594 - protein, 36 aa, Human immunodeficiency virus 2
MQIIREIINEEAADWDQQHPSPGPMPAGQLREPRGS

SEQ ID NO: 595 - protein, 36 aa, Simian immunodeficiency virus
MQIIREIINEEAADWDLQHPQPGPLPAGQLREPRGS

SEQ ID NO: 596 - protein, 36 aa, Simian immunodeficiency virus
MQIIREIINEEAADWDLQHPQPGPIPPGQLREPRGS

SEQ ID NO: 597 - protein, 35 aa, Simian immunodeficiency virus
MQIIRDIINEEAADWDLQHPQPAPQQGQLREPSGS

SEQ ID NO: 598 - protein, 35 aa, Simian immunodeficiency virus; identical to SEQ ID NO: 597

SEQ ID NO: 599 - protein, 35 aa, Simian immunodeficiency virus; identical to SEQ ID NO: 597

SEQ ID NO: 600 - protein, 35 aa, Simian immunodeficiency virus; identical to SEQ ID NO: 597

SEQ ID NO: 601 - protein, 35 aa, Simian immunodeficiency virus
MQIIRDIINEEAADWDLQHPQQAPQQGQLREPSGS

SEQ ID NO: 602 - protein, 35 aa, Simian immunodeficiency virus; identical to SEQ ID NO: 597

SEQ ID NO: 603 - protein, 35 aa, Simian immunodeficiency virus; identical to SEQ ID NO: 597

SEQ ID NO: 604 - protein, 36 aa, Simian immunodeficiency virus
MQIIREIINEEAADWDVQHPQPGPLPAGQLREPSGS

SEQ ID NO: 605 - protein, 24 aa, Human immunodeficiency virus 1
AAMQMLKDTINDEAAEWDRLHPVH

SEQ ID NO: 606 - protein, 25 aa, Human immunodeficiency virus 1
IILGLNKIVRMYSPVSILDIKQGPK

SEQ ID NO: 607 - protein, 26 aa, Human immunodeficiency virus 1
DRFFKTLRAEQATQEVKNWMTDTLLV

SEQ ID NO: 608 - protein, 24 aa, Human immunodeficiency virus 1
AAMQMLKDTINDEAAEWDRIHPQQ

SEQ ID NO: 609 - protein, 25 aa, Human immunodeficiency virus 1
IILGLNKIVRMYSPVSILDIKQGPK

SEQ ID NO: 610 - protein, 26 aa, Human immunodeficiency virus 1
DRFFKTLRAEQATQEVKGWMTDTLLV

SEQ ID NO: 611 - protein, 24 aa, Human immunodeficiency virus 1
AAMQMLKDTINEEAADWDRVHPVH

SEQ ID NO: 612 - protein, 25 aa, Human immunodeficiency virus 1
IILGLNKIVRMYSPVSILDIKQGPK

SEQ ID NO: 613 - protein, 26 aa, Human immunodeficiency virus 1
DRFFKTLRAEQATQEVKNWMTETLLV

SEQ ID NO: 614 - protein, 24 aa, Human immunodeficiency virus 1
AAMQMLKDTINEEAAEWDRVHPVH

SEQ ID NO: 615 - protein, 25 aa, Human immunodeficiency virus 1
IILGLNKIVRMYSPVSILDIKQGPK

SEQ ID NO: 616 - protein, 26 aa, Human immunodeficiency virus 1
DRFFKTLRAEQATQDVKNWMTETLLV

SEQ ID NO: 617 - protein, 24 aa, Human immunodeficiency virus 1
AAMQMLKDTINEEAAEWDRLHPVH

SEQ ID NO: 618 - protein, 25 aa, Human immunodeficiency virus 1
IILGLNKIVRMYSPVSILDIKQGPK

SEQ ID NO: 619 - protein, 26 aa, Human immunodeficiency virus 1
DRFFKTLRAEQATQDVKNWMTDTLLV

SEQ ID NO: 620 - protein, 24 aa, Human immunodeficiency virus 1
AAMQMLKDTINEEAAEWDRLHPTQ

SEQ ID NO: 621 - protein, 25 aa, Human immunodeficiency virus 1
IILGLNKIVRMYSPVGILDIKQGPK

SEQ ID NO: 622 - protein, 26 aa, Human immunodeficiency virus 1
DRFFKTLRAEQATQEVKGWMTDTLLV

SEQ ID NO: 623 - protein, 24 aa, Human immunodeficiency virus 1
AAMQMLKDTINEEAAEWDRMHPVQ

SEQ ID NO: 624 - protein, 25 aa, Human immunodeficiency virus 1
IILGLNKIVRMYSPVSILDIKQGPK

SEQ ID NO: 625 - protein, 26 aa, Human immunodeficiency virus 1
DRFFRVLRAEQATQEVKNWMTETLLV

SEQ ID NO: 626 - protein, 24 aa, Human immunodeficiency virus 1
AAMQMLKDTINEEAAEWDRLHPVH

SEQ ID NO: 627 - protein, 25 aa, Human immunodeficiency virus 1
IILGLNKIVRMYSPVSILDIKQGPK

SEQ ID NO: 628 - protein, 26 aa, Human immunodeficiency virus 1
DRFFKTLRAEQATQDVKNWMTDTLLV

SEQ ID NO: 629 - protein, 24 aa, Human immunodeficiency virus 1
AAMQMLKDTINEEAAEWDRLHPVH

SEQ ID NO: 630 - protein, 25 aa, Human immunodeficiency virus 1
IILGLNKIVRMYSPVSILDIKQGPK

SEQ ID NO: 631 - protein, 26 aa, Human immunodeficiency virus 1
DRFFKTLRAEQATQEVKGWMTETLLV

SEQ ID NO: 632 - protein, 24 aa, Human immunodeficiency virus 1
AAMQMLKDTINEEAAEWDRLHPVH

SEQ ID NO: 633 - protein, 25 aa, Human immunodeficiency virus 1
IILGLNKIVRMYSPVSILDIKQGPK

SEQ ID NO: 634 - protein, 26 aa, Human immunodeficiency virus 1
DRFFKTLRAEQATQEVKNWMTETLLV

SEQ ID NO: 635 - protein, 24 aa, Human immunodeficiency virus 1
AAMQMLKDTINEEAAEWDRVHPVH

SEQ ID NO: 636 - protein, 25 aa, Human immunodeficiency virus 1
IILGLNKIVRMYSPVSILDIKQGPK

SEQ ID NO: 637 - protein, 26 aa, Human immunodeficiency virus 1
DRFFKALRAEQATQDVKNWMTDTLLV

SEQ ID NO: 638 - protein, 24 aa, Human immunodeficiency virus 1
AAMQMLKDTINEEAAEWDRVHPVH

SEQ ID NO: 639 - protein, 25 aa, Human immunodeficiency virus 1
IILGLNKIVRMYSPVSILDIKQGPK

SEQ ID NO: 640 - protein, 26 aa, Human immunodeficiency virus 1
DRFFKALRAEQATQDVKNWMTDTLLV

SEQ ID NO: 641 - protein, 24 aa, Human immunodeficiency virus 1
AAMQMLKDTINEEAAEWDRMHPQQ

SEQ ID NO: 642 - protein, 25 aa, Human immunodeficiency virus 1
IILGLNKIVRMYSPVSILDIKQGPK

SEQ ID NO: 643 - protein, 26 aa, Human immunodeficiency virus 1
DRFFKCLRAEQASQDVKGWMTDTLLV

SEQ ID NO: 644 - protein, 24 aa, Human immunodeficiency virus 1
AAMQMLKDTINEEAAEWDRLHPVH

SEQ ID NO: 645 - protein, 25 aa, Human immunodeficiency virus 1
IILGLNKIVRMYSPVSILDIKQGPK

SEQ ID NO: 646 - protein, 26 aa, Human immunodeficiency virus 1
DRFFKTLRAEQATQDVKNWMTETLLV

SEQ ID NO: 647 - protein, 24 aa, Human immunodeficiency virus 1
AAMQMLKDTINEEAAEWDRLHPVH

SEQ ID NO: 648 - protein, 25 aa, Human immunodeficiency virus 1
IILGLNKIVRMYSPVSILDIKQGPK

SEQ ID NO: 649 - protein, 26 aa, Human immunodeficiency virus 1
DRFFRVLRAEQATQDVKNWMTDTLLV

SEQ ID NO: 650 - protein, 24 aa, Human immunodeficiency virus 1
AAMQMLKDTINEEAAEWDRLHPVH

SEQ ID NO: 651 - protein, 25 aa, Human immunodeficiency virus 1
IILGLNKIVRMYSPVSILDIKQGPK

SEQ ID NO: 652 - protein, 26 aa, Human immunodeficiency virus 1
DRFFKVLRAEQASQDVKGWMTDTLLV

SEQ ID NO: 653 - protein, 24 aa, Human immunodeficiency virus 1
AAMQMLKETINDEAAEWDRLHPVH

SEQ ID NO: 654 - protein, 25 aa, Human immunodeficiency virus 1
IILGLNKIVRMYSPVSILDIKQGPK

SEQ ID NO: 655 - protein, 26 aa, Human immunodeficiency virus 1
DRFYKTLRAEQASQDVKNWMTETLLV

SEQ ID NO: 656 - protein, 24 aa, Human immunodeficiency virus 1
AAMQMLKETINEEAAEWDRVHPVH

SEQ ID NO: 657 - protein, 25 aa, Human immunodeficiency virus 1
IILGLNKIVRMYSPTSILDIKQGPK

SEQ ID NO: 658 - protein, 26 aa, Human immunodeficiency virus 1
DRFYKTLRAEQASQDVKNWMTETLLV

SEQ ID NO: 659 - protein, 24 aa, Human immunodeficiency virus 1
AAMQMLKETINEEAAEWDRVHPVH

SEQ ID NO: 660 - protein, 25 aa, Human immunodeficiency virus 1
IILGLNKIVRMYSPTSILDIKQGPK

SEQ ID NO: 661 - protein, 26 aa, Human immunodeficiency virus 1
DRFYKTLRAEQASQEVKNWMTETLLV

SEQ ID NO: 662 - protein, 24 aa, Human immunodeficiency virus 1
AAMQMLKETINEEAAEWDRVHPVH

SEQ ID NO: 663 - protein, 25 aa, Human immunodeficiency virus 1
IILGLNKIVRMYSPTSILDIKQGPK

SEQ ID NO: 664 - protein, 26 aa, Human immunodeficiency virus 1
DRFYKTLRAEQASQEVKNWMTETLLV

SEQ ID NO: 665 - protein, 24 aa, Human immunodeficiency virus 1
AAMQMLKETINEEAAEWDRLHPVQ

SEQ ID NO: 666 - protein, 25 aa, Human immunodeficiency virus 1
IILGLNKIVRMYSPSSILDIKQGPK

SEQ ID NO: 667 - protein, 26 aa, Human immunodeficiency virus 1
DRFYKTLRAEQASQEVKNWMTETLLV

SEQ ID NO: 668 - protein, 24 aa, Human immunodeficiency virus 1
AAMQMLKETINEEAAEWDRVHPVH

SEQ ID NO: 669 - protein, 25 aa, Human immunodeficiency virus 1
IILGLNKIVRMYSPTSILDIKQGPK

SEQ ID NO: 670 - protein, 26 aa, Human immunodeficiency virus 1
DRFYKTLRAEQASQEVKNWMTETLLV

SEQ ID NO: 671 - protein, 24 aa, Human immunodeficiency virus 1
AAMQMLKETINEEAAEWDRLHPVH

SEQ ID NO: 672 - protein, 25 aa, Human immunodeficiency virus 1
IILGLNKIVRMYSPISILDIKQGPK

SEQ ID NO: 673 - protein, 26 aa, Human immunodeficiency virus 1
DRFYKTLRAEQASQEVKNWMTETLLV

SEQ ID NO: 674 - protein, 24 aa, Human immunodeficiency virus 1
AAMQMLKETINEEAAEWDRLHPVH

SEQ ID NO: 675 - protein, 25 aa, Human immunodeficiency virus 1
IIVGLNKIVRMYSPVSILDIKQGPK

SEQ ID NO: 676 - protein, 26 aa, Human immunodeficiency virus 1
DRFYKTLRAEQASQDVKNWMTETLLV

SEQ ID NO: 677 - protein, 24 aa, Human immunodeficiency virus 1
AAMQMLKETINEEAAEWDRVHPVH

SEQ ID NO: 678 - protein, 25 aa, Human immunodeficiency virus 1
IILGLNKIVRMYSPTSILDIKQGPK

SEQ ID NO: 679 - protein, 26 aa, Human immunodeficiency virus 1
DRFYKTLRAEQASQEVKNWMTETLLV

SEQ ID NO: 680 - protein, 24 aa, Human immunodeficiency virus 1
AAMQMLKETINEEAAEWDRLHPAQ

SEQ ID NO: 681 - protein, 25 aa, Human immunodeficiency virus 1
IILGLNKIVRMYSPSSILDIKQGPK

SEQ ID NO: 682 - protein, 26 aa, Human immunodeficiency virus 1
DRFYKTLRAEQASQEVKNWMTETLLV

SEQ ID NO: 683 - protein, 24 aa, Human immunodeficiency virus 1
AAMQMLKETINEEAAEWDRLHPVH

SEQ ID NO: 684 - protein, 25 aa, Human immunodeficiency virus 1
IILGLNKIVRMYSPVSILDIKQGPK

SEQ ID NO: 685 - protein, 26 aa, Human immunodeficiency virus 1
DRFYKTLRAEQATQEVKNWMTETLLV

SEQ ID NO: 686 - protein, 24 aa, Human immunodeficiency virus 1
AAMQMLKETINEEAAEWDRVHPVH

SEQ ID NO: 687 - protein, 25 aa, Human immunodeficiency virus 1
IILGLNKIVRMYSPTSILDIKQGPK

SEQ ID NO: 688 - protein, 26 aa, Human immunodeficiency virus 1
DRFYKTLRAEQASQEVKNWMTETLLV

SEQ ID NO: 689 - protein, 24 aa, Human immunodeficiency virus 1
AAMQMLKETINEEAAEWDRLHPAH

SEQ ID NO: 690 - protein, 25 aa, Human immunodeficiency virus 1
IILGLNKIVRMYSPSSILDIKQGPK

SEQ ID NO: 691 - protein, 26 aa, Human immunodeficiency virus 1
DRFYKTLRAEQASQEVKNWMTETLLV

SEQ ID NO: 692 - protein, 24 aa, Human immunodeficiency virus 1
AAMQMLKETINEEAAEWDRLHPVH

SEQ ID NO: 693 - protein, 25 aa, Human immunodeficiency virus 1
IILGLNKIVRMYSPTSILDIKQGPK

SEQ ID NO: 694 - protein, 26 aa, Human immunodeficiency virus 1
DRFYKTLRAEQASQEVKNWMTETLLV

SEQ ID NO: 695 - protein, 24 aa, Human immunodeficiency virus 1
AAMQMLKETINEEAAEWDRLHPVH

SEQ ID NO: 696 - protein, 25 aa, Human immunodeficiency virus 1
IILGLNKIVRMYSPTSILDIKQGPK

SEQ ID NO: 697 - protein, 26 aa, Human immunodeficiency virus 1
DRFYKTLRAEQASQDVKNWMTETLLV

SEQ ID NO: 698 - protein, 24 aa, Human immunodeficiency virus 1
AAMQMLKETINEEAAEWDRLHPVH

SEQ ID NO: 699 - protein, 25 aa, Human immunodeficiency virus 1
IILGLNKIVRMYSPISILDIKQGPK

SEQ ID NO: 700 - protein, 26 aa, Human immunodeficiency virus 1
DRFYKTLRAEQASQDVKNWMTETFLV

SEQ ID NO: 701 - protein, 24 aa, Human immunodeficiency virus 1
AAMQMLKETINEEAAEWDRLHPVH

SEQ ID NO: 702 - protein, 25 aa, Human immunodeficiency virus 1
IILGLNKIVRMYSPTSILDIKQGPK

SEQ ID NO: 703 - protein, 26 aa, Human immunodeficiency virus 1
DRFYKTLRAEQATQEVKNWMTETLLV

SEQ ID NO: 704 - protein, 24 aa, Human immunodeficiency virus 1
AAMQMLKETINEEAAEWDRLHPVH

SEQ ID NO: 705 - protein, 25 aa, Human immunodeficiency virus 1
IILGLNKIVRMYSPTSILDIKQGPK

SEQ ID NO: 706 - protein, 26 aa, Human immunodeficiency virus 1
DRFYKTLRAEQASQEVKNWMTETLLV

SEQ ID NO: 707 - protein, 24 aa, Human immunodeficiency virus 1
AAMQMLKETINEEAAEWDRLHPVH

SEQ ID NO: 708 - protein, 25 aa, Human immunodeficiency virus 1
IILGLNKIVRMYSPVSILDIKQGPK

SEQ ID NO: 709 - protein, 26 aa, Human immunodeficiency virus 1
DRFYKTLRAEQASQEVKGWMTETLLV

SEQ ID NO: 710 - protein, 24 aa, Human immunodeficiency virus 1
AAMQMLKEVINEEAADWDRTHPVP

SEQ ID NO: 711 - protein, 25 aa, Human immunodeficiency virus 1
IVLGLNRIVRMYSPVSILEIKQGPK

SEQ ID NO: 712 - protein, 26 aa, Human immunodeficiency virus 1
DRFYKTLRAEQATQEVKNWMTETLLV

SEQ ID NO: 713 - protein, 24 aa, Human immunodeficiency virus 1
GALQVLKEVINEEAAEWDRTHPPA

SEQ ID NO: 714 - protein, 25 aa, Human immunodeficiency virus 1
IVLGLNKMVKMYSPVSILDIKQGPK

SEQ ID NO: 715 - protein, 26 aa, Human immunodeficiency virus 1
DRFYKTLRAEQATQEVKNWMTETLLV

SEQ ID NO: 716 - protein, 24 aa, Human immunodeficiency virus 1
AAMQMLKEVINEEAAEWDRTHPAP

SEQ ID NO: 717 - protein, 25 aa, Human immunodeficiency virus 1
IVLGLNRIVRMYSPVSILEIKQGPK

SEQ ID NO: 718 - protein, 26 aa, Human immunodeficiency virus 1
DRFYKTLRAEQATQDVKNWMTETLLV

SEQ ID NO: 719 - protein, 24 aa, Human immunodeficiency virus 1
GALQVLKEVINEEAVEWDRTHPPP

SEQ ID NO: 720 - protein, 25 aa, Human immunodeficiency virus 1
IVLGLNKMVKMYSPVSILDIKQGPK

SEQ ID NO: 721 - protein, 26 aa, Human immunodeficiency virus 1
DRFYKTLRAEQATQEVKNWMTETLLV

SEQ ID NO: 722 - protein, 24 aa, Human immunodeficiency virus 2
AAMQIIREIINEEAADWDANHPIP

SEQ ID NO: 723 - protein, 25 aa, Human immunodeficiency virus 2
IQIGLQKCVRMYNPTNILDIKQGPK

SEQ ID NO: 724 - protein, 26 aa, Human immunodeficiency virus 2
DRFYKSLRAEQTDPAVKNWMTQTLLV

SEQ ID NO: 725 - protein, 24 aa, Human immunodeficiency virus 2
AAMQIIREIINEEAADWDQQHPSP

SEQ ID NO: 726 - protein, 25 aa, Human immunodeficiency virus 2
IQLGLQKCVRMYNPTNILDIKQGPK

SEQ ID NO: 727 - protein, 26 aa, Human immunodeficiency virus 2
DRFYKSLRAEQTDPAVKNWMTQTLLI

SEQ ID NO: 728 - protein, 24 aa, Human immunodeficiency virus 2
AAMQIIREIINEEAADWDQQHPSP

SEQ ID NO: 729 - protein, 25 aa, Human immunodeficiency virus 2
IQLGLQKCVRMYNPTNILDIKQGPK

SEQ ID NO: 730 - protein, 26 aa, Human immunodeficiency virus 2
DRFYKSLRAEQTDPAVKNWMTQTLLI

SEQ ID NO: 731 - protein, 24 aa, Human immunodeficiency virus 2
AAMQIIREIINEEAAEWDVQHPIP

SEQ ID NO: 732 - protein, 25 aa, Human immunodeficiency virus 2
IQIGLQKCVRMYNPTNILDIKQGPK

SEQ ID NO: 733 - protein, 26 aa, Human immunodeficiency virus 2
DRFYKSLRAEQTDPAVKNWMTQTLLV

SEQ ID NO: 734 - protein, 24 aa, Human immunodeficiency virus 2
AAMQIIREIINEEAADWDVQHPIP

SEQ ID NO: 735 - protein, 25 aa, Human immunodeficiency virus 2
IQIGLQKCVRMYNPTNILDIKQGPK

SEQ ID NO: 736 - protein, 26 aa, Human immunodeficiency virus 2
DRFYKSLRAEQTDAAVKNWMTQTLLV

SEQ ID NO: 737 - protein, 24 aa, Human immunodeficiency virus 2
AAMQIIREIINEEAADWDAQHPIP

SEQ ID NO: 738 - protein, 25 aa, Human immunodeficiency virus 2
IQIGLQKCVRMYNPTNILDIKQGPK

SEQ ID NO: 739 - protein, 26 aa, Human immunodeficiency virus 2
DRFYKSLRAEQTDPAVKNWMTQTLLV

SEQ ID NO: 740 - protein, 24 aa, Human immunodeficiency virus 2
AAMQIIREIINEEAADWDQQHPIP

SEQ ID NO: 741 - protein, 25 aa, Human immunodeficiency virus 2
IQIGLQKCVRMYNPTNILDIKQGPK

SEQ ID NO: 742 - protein, 26 aa, Human immunodeficiency virus 2
DRFYKSLRAEQTDPAVKNWMTQTLLI

SEQ ID NO: 743 - protein, 24 aa, Human immunodeficiency virus 2
AAMQIIREIINEEAADWDVAHPIP

SEQ ID NO: 744 - protein, 25 aa, Human immunodeficiency virus 2
IQIGLQKCVRMYNPTNILDIKQGPK

SEQ ID NO: 745 - protein, 26 aa, Human immunodeficiency virus 2
DRFYKSLRAEQTDPAVKNWMTQTLLI

SEQ ID NO: 746 - protein, 24 aa, Human immunodeficiency virus 2
AAMQIIREIINEEAADWDAQHPIP

SEQ ID NO: 747 - protein, 25 aa, Human immunodeficiency virus 2
IQIGLQKCVRMYNPTNILDVKQGPK

SEQ ID NO: 748 - protein, 26 aa, Human immunodeficiency virus 2
DRFYKSLRAEQTDPAVKNWMTQTLLI

SEQ ID NO: 749 - protein, 24 aa, Human immunodeficiency virus 2
AAMQIIREIINDEAADWDAQHPIP

SEQ ID NO: 750 - protein, 25 aa, Human immunodeficiency virus 2
IQIGLQKCVRMYNPTNILDVKQGPK

SEQ ID NO: 751 - protein, 26 aa, Human immunodeficiency virus 2
DRFYKSLRAEQTDPAVKNWMTQTLLI

SEQ ID NO: 752 - protein, 24 aa, Human immunodeficiency virus 2
AAMQIIREIINEEAADWDVQHPIP

SEQ ID NO: 753 - protein, 25 aa, Human immunodeficiency virus 2
IQIGLQKCVRMYNPTNILDVKQGPK

SEQ ID NO: 754 - protein, 26 aa, Human immunodeficiency virus 2
DRFYKSLRAEQTDPAVKNWMTQTLLV

SEQ ID NO: 755 - protein, 24 aa, Human immunodeficiency virus 2
AAMQIIREIINEEAADWDSQHPIP

SEQ ID NO: 756 - protein, 25 aa, Human immunodeficiency virus 2
IQIGLQKCVRKYNPTNILDIKQGPK

SEQ ID NO: 757 - protein, 26 aa, Human immunodeficiency virus 2
DRFYKSLRAEQTDPAVKNWMTQTLLI

(19) United States
(12) Patent Application Publication
(10) Pub. No.: US 2026/0056196 A1
(43) Pub. Date: Feb. 26, 2026

(54) DETECTION OF ANTIGENS

(71) Applicant: ARIZONA BOARD OF REGENTS ON BEHALF OF ARIZONA STATE UNIVERSITY, Scottsdale, AZ (US)

(72) Inventors: Ye Hu, Scottsdale, AZ (US); Xiangxing Kong, Scottsdale, AZ (US); Tanxi Cai, Scottsdale, AZ (US); Qingbo Shu, Scottsdale, AZ (US)

(21) Appl. No.: 19/248,129

(22) Filed: Jun. 24, 2025

Related U.S. Application Data
(62) Division of application No. 17/617,802, now Pat. No. 12,385,915.
(60) Provisional application No. 62/861,896, filed on Jun. 14, 2019.

(51) Int. Cl. G01N 33/569; G01N 33/543; G01N 33/68

(52) U.S. Cl. CPC G01N 33/569; G01N 33/54326; G01N 33/6848; G01N 2333/35; G01N 2446/00; G01N 2458/15

(57) ABSTRACT

Provided herein are methods for detecting and identifying discase-specific biomarkers, such as target antigens associated with infection.

This application a divisional of a pending U.S. application Ser. No. 17/617,802, filed on Dec. 9, 2021, which is a national phase application under 35 C.F.R. § 371 of PCT Application PCT/US2020/037785, filed on 15 Jun. 2020, which claims priority from U.S. Provisional Patent Application No. 62/861,896, filed on Jun. 14, 2019, the contents of each of which are incorporated herein by reference in their entireties.

All patents, patent applications and publications cited herein are hereby incorporated by reference in their entirety. The disclosures of these publications in their entireties are hereby incorporated by reference into this application in order to more fully describe the state of the art as known to those skilled therein as of the date of the invention described and claimed herein.

This patent disclosure contains material that is subject to copyright protection. The copyright owner has no objection to the facsimile reproduction by anyone of the patent document or the patent disclosure as it appears in the U.S. Patent and Trademark Office patent file or records, but otherwise reserves any and all copyright rights.

### GOVERNMENT INTERESTS

This invention was made with government support under R01 AI113725, R01 AI122932, and R01 HD090927 awarded by the National Institutes of Health. The government has certain rights in the invention.

### SEQUENCE LISTING

In accordance with 37 C.F.R. § 1.831, the present specification references a Sequence Listing which has been submitted electronically in XML format. The entire contents of the Sequence Listing XML are incorporated herein by reference, with the intention that, upon publication (including issuance), this incorporated Sequence Listing will be inserted in the published document immediately before the claims. Said XML file, created on Oct. 27, 2025, is named 2952332-000002-US3_SL.xml and is 674,969 bytes in size.

### FIELD OF THE INVENTION

This invention is directed to methods for detecting and identifying target antigens associated with infection.

### BACKGROUND OF THE INVENTION

Conventional methods of detecting pathogen proteins in human biological samples down are incapable of detecting sub-nanomolar levels, which is is critical for accurate infection diagnosis. Accordingly, there is a need for improved methods for infection diagnosis. The problems addressed by this disclosure include providing such improved methods.

### SUMMARY OF THE INVENTION

An aspect of the invention is directed towards a method for detection and identification of a disease-specific biomarker. In one embodiment, the method comprises contacting an enzyme-digested biological sample with an antibody-modified solid support (AMSS) under conditions that promote binding of the AMSS to its target if present in the contacted biological sample, wherein the antibodies bind specifically to a disease-specific biomarker; sensing the disease-specific biomarker in a concentration comprising the range of about 0.1 pM to about 6 μM; subjecting the sample to a mass spectrometry-based analytical technique; detecting m/z peaks in the mass spectrum; and identifying the subject from which biological sample was obtained as having the disease based on the m/z peaks in the mass spectrum. An aspect of the invention is directed towards a method of detecting and identifying a disease in a subject. In one embodiment, the method comprises identifying the disease-specific biomarker according to the technique described herein; identifying the similarities and differences between disease-specific biomarker spectrum and the spectrum obtained from the sample from the subject; identifying a subject who has contracted a disease or disorder, or who are at risk of developing an active disease or disorder, based on the m/z peaks in the mass spectrum; and treating the subject with a therapeutically effective amount of a medicament. In an embodiment, the disease is caused by an infectious pathogen comprising a bacterium, a fungus, or a virus. In a further embodiment, the bacterial disease comprises tuberculosis, nontuberculosis mycobacterial (NTM) disease, or gut microbial perturbations. In another embodiment the viral disease comprises a human immunodeficiency virus (HIV) disease or Ebola disease. In an embodiment, the disease-specific biomarker comprises a disease-specific antigen, a disease-specific protein, a disease-specific peptide, or a fragment thereof. In an embodiment, the disease-specific target biomarker has a molecular weight in the range of about 500 Daltons (Da) to about 5000 Da. In an embodiment, disease-specific biomarker is a Mycobacterium peptide comprising the sequence TDAATLAQEAGNFER (SEQ ID NO:1), TQIDQVESTAGSLQGQWR (SEQ ID NO:2), WDATATELNNALQNLAR (SEQ ID NO:3), TQIDQVESTAASLQAQWR (SEQ ID NO:4), or a combination thereof. In an embodiment, disease-specific biomarker is a HIV-1 1 specific peptide comprising the sequence ETINEEAAEWDR (SEQ ID NO: 5), DTINEEAAEWDR (SEQ ID NO: 6), MYSPTSILDIR (SEQ ID NO: 7), MYSPVSILDIK (SEQ ID NO: 8), MYSPVSILDIR (SEQ ID NO: 9), or a combination thereof. In an embodiment, the disease-specific biomarker is a HIV-2 specific peptide comprising the sequence MYNPTNILDIK (SEQ ID NO: 10), AEQTDPAVK (SEQ ID NO: 11), or a combination thereof. In an embodiment, the enzyme-digested biological sample comprises one or more internal reference standards. In a further embodiment, the internal reference standard comprises an isotopically labeled sample. In an embodiment, the biological sample is obtained from a human subject. In a further embodiment, the biological sample is blood, serum, cerebrospinal fluid, semen, urine, plasma, or a biological culture media. In an embodiment, the method further comprising generating a reference mass spectrum in which a peak corresponding to a disease-specific biomarker of interest is present in the reference mass spectrum.

In an embodiment, the AMSS is a non-porous support or a porous support. In an embodiment, the AMSS comprises a bead, a nanodisk, a microdisk, a film, rod, nanoparticle or a microparticle. In an embodiment, the AMSS is etched for structure. In an embodiment, the AMSS comprises a magnetic bead.

In an embodiment, the mass spectrometry-based analytical technique is applied to the disease-specific biomarker bound to the AMSS or is applied to an eluted disease-specific biomarker. In an embodiment, the mass spectrometry-based analytical technique comprises a hard ionization technique or a soft ionization technique. In an embodiment, the hard ionization technique comprises electronic ionization (EI). In an embodiment, the soft ionization technique comprises: matrix-assisted laser desorption/ionization (MALDI), electrospray ionization (ESI), fast atom bombardment (FAB), chemical ionization (CI), atmospheric-pressure chemical ionization (APCI), desorption electrospray ionization (DESI), atmospheric pressure photoionization (APPI), or secondary ion mass spectrometry (SIMS). In an embodiment, the hard ionization technique or the soft ionization technique is coupled with at least two mass analyzers and at least one technique to induce fragmentation comprises tandem mass spectrometry (MS2). In an embodiment, the at least one technique to induce fragmentation comprises collision-induced dissociation (CID), electron capture dissociation (ECD), electron transfer dissociation (ETD), negative electron transfer dissociation (NETD), electron-detachment dissociation (EDD), charge transfer dissociation (CTD), photodissociation, infrared multiphoton dissociation (IRMPD), blackbody infrared radiative dissociation (BIRD), or surface induced dissociation (SID). In an embodiment, the tandem mass spectrometry (MS2) technique comprises data-independent acquisition (DIA) or data-dependent acquisition (DDA).

In an embodiment, the mass spectrometry-based analytical technique comprises at least one mass analyzer. In a further embodiment, the at least one mass analyzer comprises a quadrupole mass analyzer, a time of flight (TOF) mass analyzer, a magnetic sector mass analyzer, an electrostatic sector mass analyzer, a quadrupole ion trap mass analyzer, an orbitrap mass analyzer, or ion cyclotron resonance mass analyzer. In an embodiment, the method comprises MS²/MS³data dependent neutral loss method. In an embodiment, the method comprises nanoelectrospray-tandem mass spectrometry (iNanoESI-MS/MS). In an embodiment, the hard ionization technique or the soft ionization technique is coupled with at least two mass analyzers and at least one technique to induce fragmentation comprises tandem mass spectrometry (MS²).

In an embodiment, the mass spectrometry-based analytical technique is combined with a separation technique. In a further embodiment, the mass spectrometry-based analytical technique combined with a separation technique comprises liquid chromatography mass spectrometry (LC-MS), liquid chromatography with tandem mass spectrometry (LC-MS-MS or LC-MS²), liquid chromatography scheduled parallel reaction monitoring mass spectrometry (LC-sPRM-MS), NanoLC-ESI-MS/MS or immunoprecipitation coupled liquid chromatography scheduled parallel reaction monitoring mass spectrometry (LC-iSPRM-MS).

In an embodiment, the m/z peaks [M+H]⁺ identified by MALDI-TOF MS comprise: a m/z peak at 1594 is indicative of a disease-specific target antigen TDAATLAQEAGNFER (SEQ ID NO: 1) associated with co-infection by Mycobacterium tuberculosis complex subspecies and one or more of M. kansasii, M. marinum, and M. ulcerans infection; a m/z peak at 1901 (ESAT-6) is indicative of a disease-specific target antigen WDATATELNNALQNLAR (SEQ ID NO: 3) associated with infection by Mycobacterium tuberculosis complex species; a m/z peak at 2004 is indicative of a disease-specific target antigen TQIDQVESTAGSLQGQWR (SEQ ID NO: 2) associated with infection by Mycobacterium tuberculosis complex subspecies; a m/z peak at 2032 is indicative of a disease-specific target antigen associated with infection by M. kansasii; or a combination thereof.

In another embodiment, m/z peaks [M+H]⁺ identified by MALDI-TOF MS comprise: a m/z peak at 1463 Da is indicative of a disease-specific peptide ETINEEAAEWDR (SEQ ID NO: 5) of HIV-1 infection; a m/z peak at 1448 Da is indicative of a disease-specific peptide DTINEEAAEWDR (SEQ ID NO: 6) of HIV-1 infection; a m/z peak at 1294 Da is indicative of a disease-specific peptide MYSPVSILDIR (SEQ ID NO: 9) of HIV-1 infection; a m/z peak at 1296 Da is indicative of a disease-specific peptide MYSPTSILDIR (SEQ ID NO: 7) of HIV-1 infection; or a combination thereof.

In another embodiment, m/z peaks [M+H]⁺ identified by MALDI-TOF MS comprise: a m/z peak at 1322 Da is indicative of a disease specific peptide MYNPTNILDIK (SEQ ID NO: 10) of HIV-2 infection; a m/z peak at 958 Da is indicative of a disease specific peptide AEQTDPAVK (SEQ ID NO: 11) indicative of HIV-2 infection; or a combination thereof.

An aspect of the invention is directed towards a kit for the detection and identification of a biomarker of disease or disorder, the kit comprising one or more of antibody-modified solid supports, wherein the antibodies bind specifically to a disease or disorder peptide or fragment thereof and instructions for use.

### BRIEF DESCRIPTION OF THE DRAWINGS

FIG. 1A-D show schematic illustrations of embodients of an exemplary magnetic NS MALDI-MS platform to detect peptide sequences from tryptic digestion of CFP10 in human serum or plasma.

FIG. 2 is a SEM image of magnetic nanoparticles (NPs).

FIG. 3 presents titration curves for CFP-10 (peptide 1593) quantitation in serum. MALDI-TOF MS spectra of healthy serum spiked with CFP-10 after microwave-assisted trypsin digestion. Peptide 1593 was captured by anti-1593 antibody modified NPs. The Limit of Detection was 0.26 nM in serum. Internal isotope peptide (MW: 1604), reference: 1 nM, relative to 100 uL serum, added after tryptic digestion.

FIG. 4 presents MALDI-TOF MS spectra of healthy serum spiked with CFP-10 after microwave-assisted trypsin digestion. Peptide 2004 were captured by antibody-2004 modified NPs. The Limit of Detection was 0.4 nM in serum. Internal isotope peptide (MW: 2014), reference: 1 nM, relative to 100 uL serum, added after tryptic digestion.

FIGS. 5A-5D demonstrate origination of selected peptide sequences after tryptic digestion of CFP-10 and ESat-6, obtained at unipept.ugent.be on the World Wide Web. Figures disclose SEQ ID NOS 1-4, respectively, in order of appearance.

FIGS. 6A-6C show the workflow for MRM-MS detection of CFP-10 peptide in serum. (FIG. 6A) Sample processing prior to MS analysis. (FIG. 6B) Scheme of LC-MRM MS system and the flow setting. Buffers that are used for eluting peptides from analytical column are colored red. Buffer that is used for sample loading is colored orange. This is cited from the instrument manual of Thermo Scientific UltiMate 3000 RSLCnano system with minor modification. (FIG. 6C) The MRM-MS signal that is generated from the targeted peptide (red) and its internal standard with stable isotopic labeling (green), which causes 10 Da of mass shift in its product fragments.

FIGS. 7A-7C show representative MS signal from a positive clinical sample. (FIG. 7A) The intensity of each MRM transitions selected for a targeted CFP-10 peptide (upper panel) and its internal standard (lower panel) are presented, and their m/z are labeled on each centroid peak. (FIG. 7B) The extracted ion chromatograph of the targeted CFP-10 peptide exported from Skyline. (FIG. 7C) The extracted ion chromatograph of internal standard peptide that is generated by stable isotopic labeling of targeted CFP-10 peptide. All monitored fragments are presented with different colors and their m/z and charge are listed in the box legend.

FIGS. 8A-8C present Selection of MRM transitions. (FIG. 8A) Overview of all theoretical b and y ions of the targeted CFP-10 peptide with m/z in the range of 300-1,500, and their human proteome interferences provided by SRM Collider. All ions selected in the final assay are marked with green circles, and some ions that are not selected due to the criteria setting illustrated in FIG. 10 are marked with red circles. (FIG. 8B) The MS/MS spectrum of targeted peptide are identified after database search and used for spectral library build-up in Skyline. (FIG. 8C) The retention times and mass-to-charge ratios of y13 (left panel, red dot) and y9 (right panel, red dot) fragment ions from targeted peptide and their interferences (blue dots). FIGS. 8B and 8C disclose “TDAATLAQEAGNFER” as SEQ ID NO: 1.

FIG. 9 presents Scheme 1 for pathogen selection, method development, and method validation.

FIG. 10 presents Box 1, a scheme of how to select the MRM transitions via machine based.

FIG. 11 presents a schematic illustration of the iPRM-MS platform. Serum from HIV were trypsin digested, spiked with stable isotope-labeled internal standards, antibody-enriched for the two target peptides, and analyzed by LC-SPRM-MS. Multiplex quantification of target peptides is determined by MS intensity ratio of target and isotope labeled internal standard peptides.

FIG. 12 shows a sequence alignment of Gap proteins from different HIV-1 and HIV-2 strains (SEQ ID NOS 605-757, respectively, in order of appearance). Sequences of HIV-1 and HIV-2 from the UniProtKB database were aligned by CLUSTAL Omega (1.2.4) multiple sequence alignment (http://www.uniprot.org/align/). 4 peptides the target peptides with corresponding sequence regions in from HIV-1 (SEQ ID NOS 5, 6, 9, and 7, respectively, in order of appearance) and 2 peptides from HIV-2 (SEQ ID NOS 10 and 11, respectively, in order of appearance) are selected to be our target peptides used for the detection and quantification of HIV-1 and HIV-2, respectively, based on their specificity. The combination of 4 peptides from HIV-1 allows for the detection of more than 95% of HIV-1 strains, while the combination of 2 peptides can cover more than 98% percent of HIV-2 strains. Red text (R and K) in the aligned sequence indicates tryptic cleavage sites.

FIG. 13 shows the quantification of both HIV antigen peptides (P24 peptides) and Mtb antigen peptides (CFP-10 peptides) in the same spectrum. This allows for the diagnosis and monitoring of the pathological status of HIV-Tb co-infected patients from a single biological same using the same mass spectrometry quantification spectrum.

FIG. 14 shows an instrument method.

FIG. 15 shows a MALDI-TOF MS spectrum of recombinant p24 tryptic digestion products.

FIG. 16 shows a phylogenic tree of the HIV and SIV viruses. The image indicates the major groupings of HIV and SIV viruses. The HIV-1 M (“major”) group contains HIV-1 subtypes responsible for most human infections, with the subtypes in this group accounting for >90% of reported HIV/AIDS cases, although the prevalence of these subtypes and their circulating recombinant forms (CRFs) varies by geographical locale (see FIG. 17).

FIG. 17 shows Global and regional molecular epidemiology of HIV-1, 1990-2015: a systematic review, global survey, and trend analysis (Hemelaar, J. et al., The Lancet Infectious Diseases, 2018, 19 (2), p. 143-155. HIV-1 M subtypes A, B and C appear to be responsible for most (72.9%) global HIV-1 infections, with HIV-1 subtype C accounting for almost half (46.6%) of all cases. Circulating recombinant forms (CRFs; subtype A derivatives), and unique recombinant forms (URFs) of HIV-1 M and were responsible for most of the remaining global HIV-1 infections (22.8% of the total). Rough distributions and prevalence of each virus are as follows: HIV-1 M Subtype A (common in Eastern Africa, Eastern Europe and Central Asia), Subtype B (major form found in North and South America, Europe, the Middle East, North Africa, Japan, and Australia), Subtype C (dominant in Southern Africa, Eastern Africa, and India), Subtype D (primarily found in Eastern and Central Africa), Subtype F (detected in Central Africa, South America and Eastern Europe) Subtype G (detected in Africa and Central Europe), Subtype H (restricted to Central Africa), and Subtype J (primarily detected in North, Central and West Africa, and the Caribbean). For the three other subtypes, while Subtype K (restricted to the Democratic Republic of Congo (DRC) and Cameroon) and Subtype L (limited to the DRC), can be new subtypes. Non-group M virus infections are rare and geographically restricted: all known HIV-1 N cases are restricted (<20 cases as of 2015), while HIV-1 O cases are primarily detected in West Central Africa (<2% of HIV-1 cases in Cameroon). Most (>80%) HIV-2 cases are detected in West Africa, and only Subtypes A and B are considered to be pandemic. (Summary adapted from S, Pattou C, Walker N, Schwardlander B, Esparza J; WHO-UNAIDS Network for HIV Isolation and Characterization. (2002) Estimated global distribution and regional spread of HIV-1 genetic subtypes in the year 2000. J Acquir Immune Defic Syndr. 29(2): 184-90).

FIG. 18A-B show LC-iSPRM-MS platform for simultaneous detection and quantification of HIV and TB. Panel a: serum from HIV-1-and TB-free human subjects was spiked with recombinant p24 and CFP-10 protein, then trypsin digested, spiked with stable isotope-labeled internal standards, antibody-enriched for the two target peptides, and analyzed by LC-SPRM-MS. Multiplex quantification of target peptides are determined by MS intensity ratio of target and isotope labeled internal standard peptides. Panel b: p24-and CFP-10-specific peptides can be simultaneously enriched and sensitively detected from spiked serum samples with high specificity due to the multiple confirmations provided by the mass spectrometer, including the retention time, mass-over-charge (m/z) and MS/MS spectrum of the target peptides.

FIG. 18C-D show LC-PRM-MS based enhancement of sensitivity and specificity and multiplex quantification.

FIG. 18E discloses the “p24” sequences as SEQ ID NO: 13 and the “CFP-10” sequences as SEQ ID NO: 1.

FIG. 19A shows quantitative detection of p24 antigen for diagnosis and treatment monitoring of HIV-1 infection. HIV-1 diagnosis using serum from adults and infants. Solid horizontal lines indicate the median and 95% confidence intervals for each group. The red dashed line indicates the p24 cut-off value (0.1 pmol/L) for HIV-1 diagnosis.

FIG. 19B shows quantitative detection of p24 antigen for diagnosis and treatment monitoring of HIV-1 infection. Quantitative monitoring of p24 antigen in seroconversion panels.

FIG. 19C shows quantitative detection of p24 antigen for diagnosis and treatment monitoring of HIV-1 infection. Longitudinal monitoring of p24 in patients receiving antiretroviral therapy from p1041 cohort.

FIG. 20A shows Simultaneous Measurement of p24 and CFP-10 in patient serum samples. Heat map of p24 (lower cell, blue) and CFP-10 (upper cell, red) levels in serum from patients with or without HIV, TB or HIV/TB infections, where each column depicts the results from an individual subject, ranked by high to low CFP-10 concentration. Color intensity reflects the triplicate mean of the antigen level and correspond the concentrations in the matching gradient bars.

FIG. 20B shows Simultaneous Measurement of p24 and CFP-10 in patient serum samples. Longitudinal monitoring of serum p24 and CFP-10 concentration in HIV-1/TB co-infected patients receiving anti-TB treatment.

FIG. 21A shows MALDI-TOF MS signals of tryptic digested p24 fragments. Mass spectra of recombinant p24 tryptic digestion products.

FIG. 21B shows MALDI-TOF MS signals of tryptic digested p24 fragments. Theoretical [M+H]⁺ values of p24 tryptic digestion products (SEQ ID NOS 334, 5, 335, 7, and 336, respectively in order of appearance).

FIG. 22A-B show LC-MS/MS spectra of target peptides from recombinant p24. The p24 precursor ions of the target peptides corresponding to 2+ charge state (m/z of 648.34 and 731.82) were chosen for MS/MS fragmentation. The b- and y-ion series indicate the sequence of (a) ETINEEAAEWDR (SEQ ID NO: 5), [M+H]²⁺ at m/z of 731.82; (b) DTINEEAAEWDR (SEQ ID NO: 6), [M+H]²⁺ at m/z of 724.82. FIG. 22B discloses SEQ ID NO: 7.

FIG. 23 shows HIV-1 and HIV-2 sequence alignment (SEQ ID NOS 605-757, respectively, in order of appearance). Sequence alignment of the p24 protein region from HIV-1 and HIV-2 UniProtKB database entries, where highlighted text indicates tryptic cleavage sites (red, R and K residues), and regions of sequence identity (dark blue) and amino acid conservation (light blue). FIG. 23 discloses the four peptide sequences in the panel beneath the alignment as SEQ ID NOS 5, 6, 9, and 7, respectively, in order of appearance.

FIG. 24 shows simultaneous enrichment of p24-speicific peptide ETINEEAAEWDR (SEQ ID NO: 5) and its variant DTINEEAAEWDR (SEQ ID NO: 6) by an antibody raised against ETINEEAAEWER (SEQ ID NO: 13) after both peptides were spiked into serum (6 pM each) and subjected to antibody enrichment. A 0.3 min of time shift was found between the two peptides of ETINEEAAEWDR (SEQ ID NO: 5) (a) and DTINEEAAEWDR (SEQ ID NO: 6) (b), which produced similar intensities. (c), (d), (e) and (f) MS/MS spectra of (c) ETINEEAAEWDR (SEQ ID NO: 5), (d) DTINEEAAEWDR (SEQ ID NO: 6), and their respective (e) (f) isotopic labeled internal standards (I.S).

FIG. 25 shows longitudinal monitoring of serum p24 and CFP-10 concentration in HIV-1/TB co-infected patients receiving anti-TB treatment.

FIG. 26 shows a chart of the values of using immobilized TPCK Trypsin instead of Sequencing grade Trypsin.

FIG. 27 shows chromatograms resulting from the use of a variety of magnetic nanoparticles from different manufacturers.

FIG. 28 shows two chromatograms before and after removing unspecific binding.

FIG. 29 shows chromatograms resulting from the use of different magnetic bead platforms and varying concentrations of the pathogen target peptide.

FIG. 30 shows chromatograms resulting from the use of two different magnetic bead platforms and varying concentrations of the pathogen target peptide generated from a Thermo TSQ Altis quadrupole mass spectrometer.

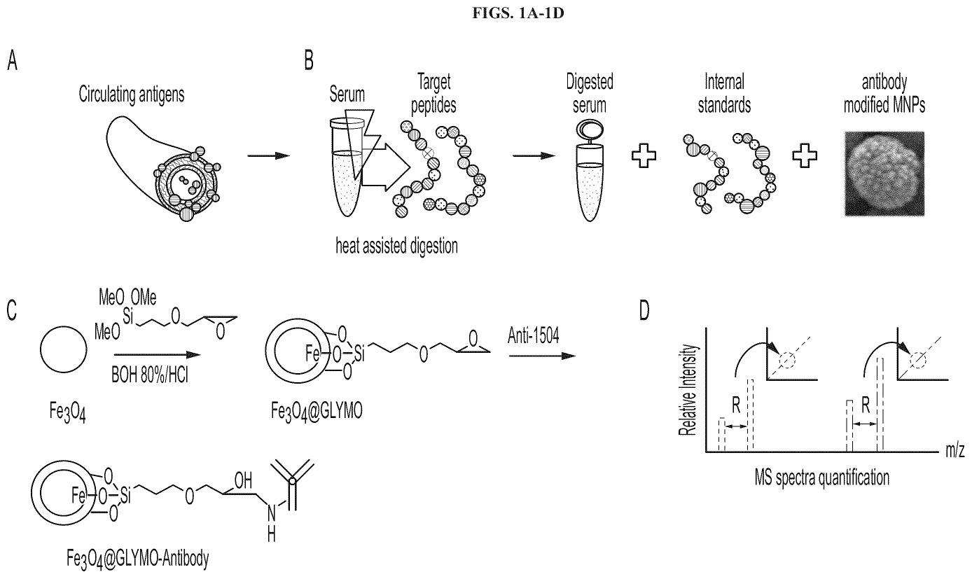

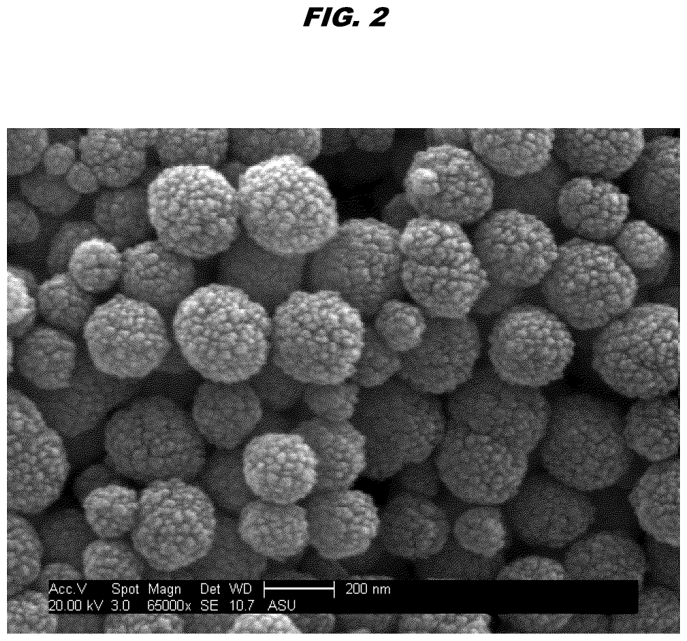

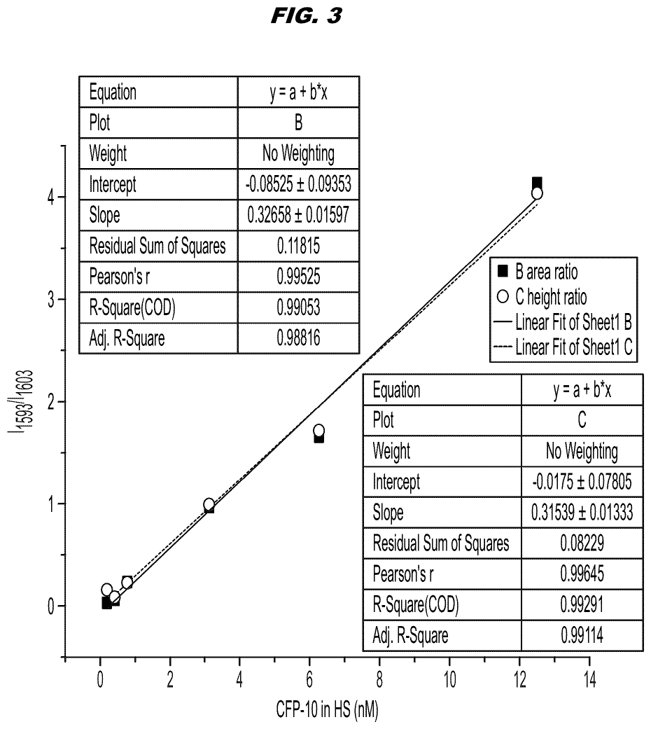

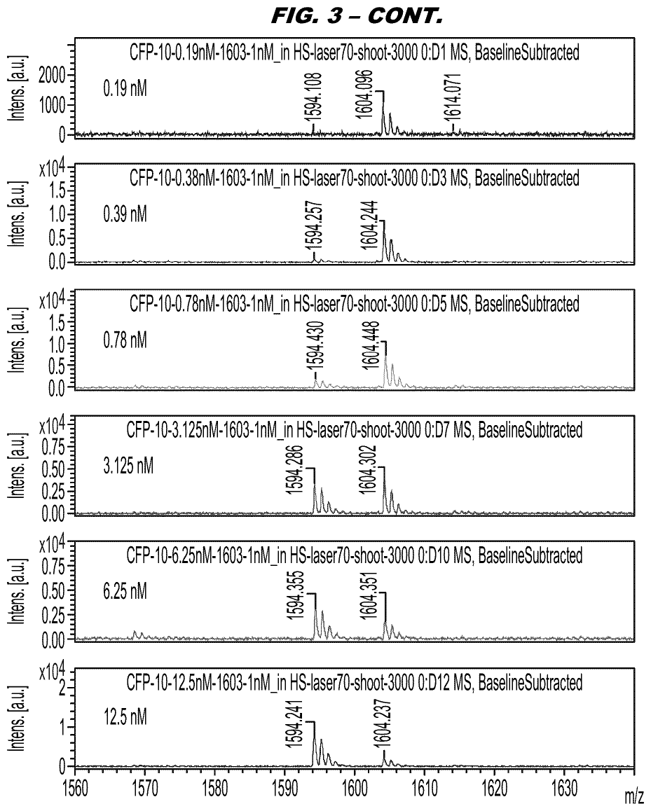

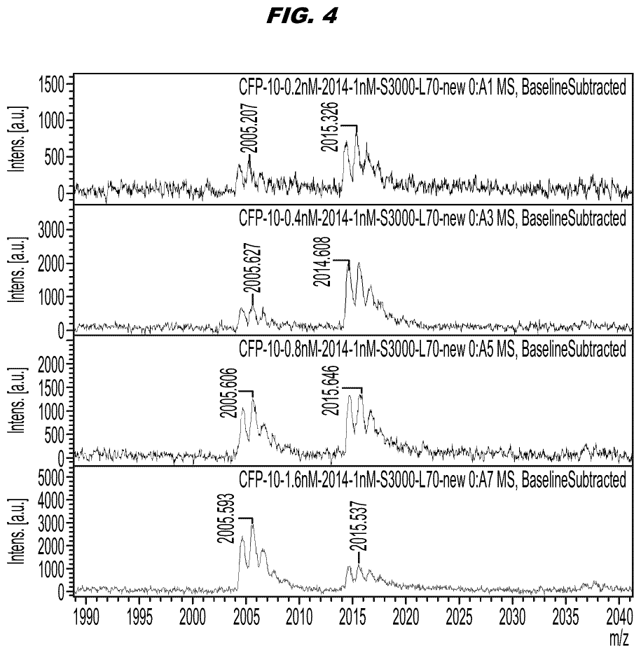

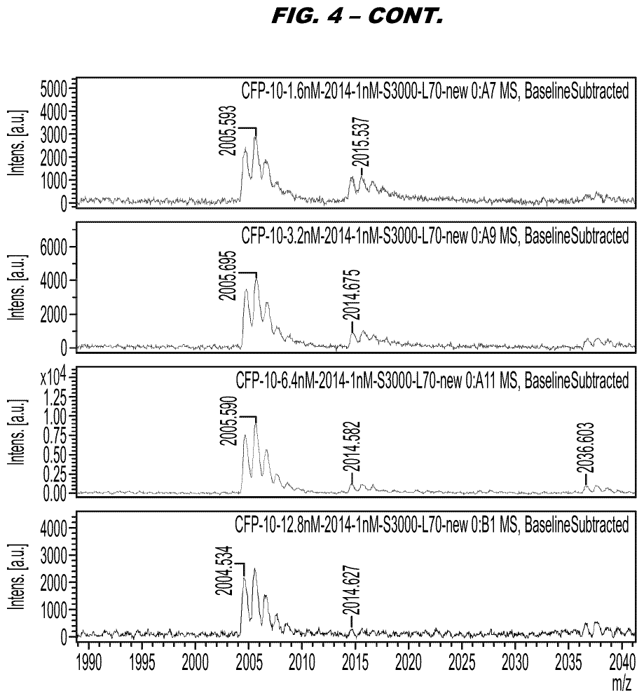

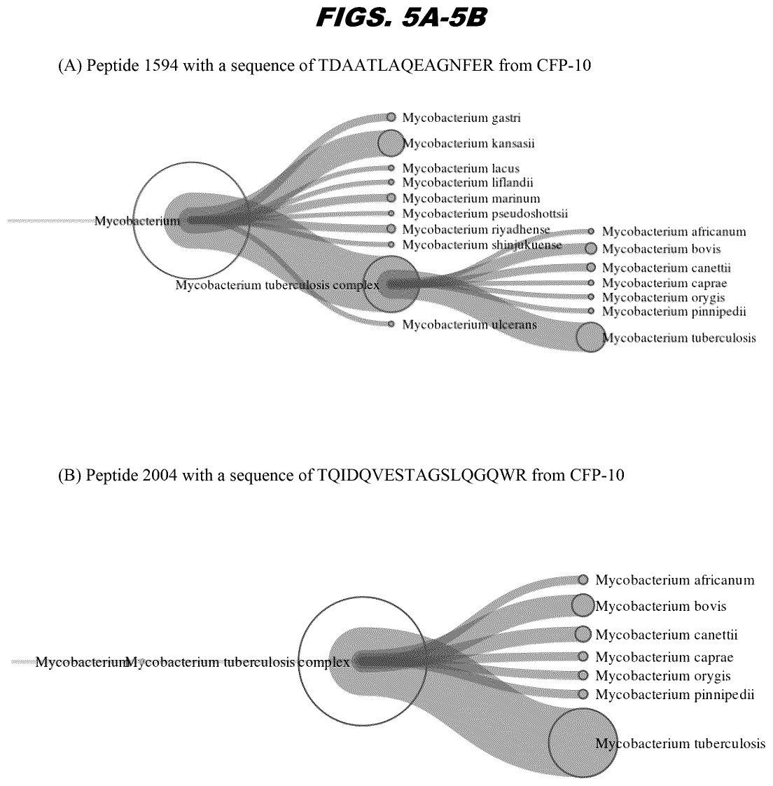

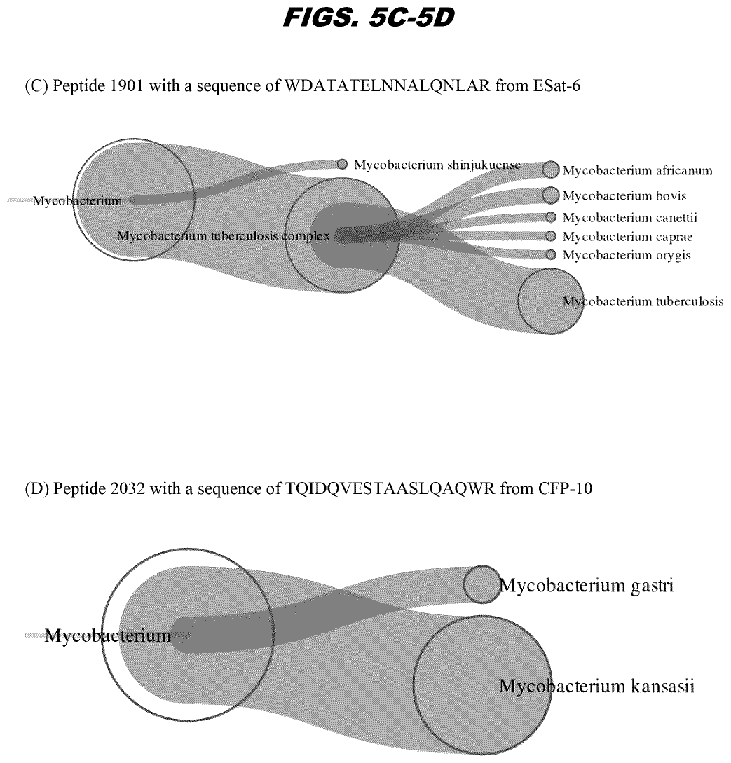

### DETAILED DESCRIPTION

The methods and compositions provided herein are based at least in part on the inventors' development of mass spectrometry-based methods for the detection and identification of a disease-specific biomarker (for example, pathogenic antigens) in a biological sample (such as serum or plasma). As described herein, the presence of particular disease-associated target antigens in the sample provides infection information. In some embodiments, the information is pertinent for tuberculosis (TB) diagnosis. In some embodiments, the information is pertinent for monitoring TB therapy. In some embodiments, the information is pertinent for HIV diagnosis. In some embodiments, the information is pertinent for monitoring HIV treatment.

Detailed descriptions of one or more embodiments are provided herein. It is to be understood, however, that the present invention may be embodied in various forms. Therefore, specific details disclosed herein are not to be interpreted as limiting, but rather as a basis for the claims and as a representative basis for teaching one skilled in the art to employ the present invention in any appropriate manner.

Unless defined otherwise, all technical and scientific terms used herein have the same meanings as commonly understood by one of ordinary skill in the art to which this invention belongs. Although any methods and materials similar or equivalent to those described herein can be used in the practice or testing of the present invention, the preferred methods and materials are now described. All publications mentioned herein are incorporated herein by reference for the purpose of describing and disclosing the chemicals, cell lines, vectors, animals, instruments, statistical analysis and methodologies which are reported in the publications which might be used in connection with the invention. Nothing herein is to be construed as an admission that the invention is not entitled to antedate such disclosure by virtue of prior invention.

The singular forms “a”, “an” and “the” include plural reference unless the context clearly dictates otherwise. The use of the word “a” or “an” when used in conjunction with the term “comprising” in the claims and/or the specification may mean “one,” but it is also consistent with the meaning of “one or more,” “at least one,” and “one or more than one.”

Wherever any of the phrases “for example,” “such as,” “including” and the like are used herein, the phrase “and without limitation” is understood to follow unless explicitly stated otherwise. Similarly, “an example,” “exemplary” and the like are understood to be nonlimiting.

The term “substantially” allows for deviations from the descriptor that do not negatively impact the intended purpose. Descriptive terms are understood to be modified by the term “substantially” even if the word “substantially” is not explicitly recited.

The terms “comprising” and “including” and “having” and “involving” (and similarly “comprises”, “includes,” “has,” and “involves”) and the like are used interchangeably and have the same meaning. Specifically, each of the terms is defined consistent with the common United States patent law definition of “comprising” and is therefore interpreted to be an open term meaning “at least the following,” and is also interpreted not to exclude additional features, limitations, aspects, etc. Thus, for example, “a process involving steps a, b, and c” means that the process includes at least steps a, b and c. Wherever the terms “a” or “an” are used, “one or more” is understood, unless such interpretation is nonsensical in context.

As used herein the term “about” is used herein to mean approximately, roughly, around, or in the region of. When the term “about” is used in conjunction with a numerical range, it modifies that range by extending the boundaries above and below the numerical values set forth. In general, the term “about” is used herein to modify a numerical value above and below the stated value by a variance of 20 percent up or down (higher or lower).

### Methods of Detection and Identification

Aspects described herein are directed towards methods of detection and identification of a disease-specific biomarker using the MS methodologies described herein. Aspects described herein are also directed towards methods of detecting and identifying a disease in a subject using the MS methodologies described herein. The term “disease-specific biomarker” can refer to any marker that indicates the presence of a disease. In some embodiments, the disease-specific biomarker comprises a disease-specific antigen, a disease-specific protein, a disease-specific peptide, or a fragment thereof. In some embodiments, the disease-specific biomarker can be 5, 6, 7, 8, 9, 10, 11, 12, 13, 14, 15, 16, 17, 18, 19, 20, 25, or 30 amino acids in length. For example, as described herein, a disease-specific biomarker can be selected based on a combination of the marker's signal intensity when analyzed by MALDI-TOF MS (for example) and the marker's ability to detect and/or discriminate HIV-1 subtypes and HIV-2. Without wishing to be bound by theory, a disease-specific biomarker (for example, a disease-specific peptide) contains a region of significant sequence conservation among the different species or strains (such as bacterial species or viral strains) that will be analyzed in the assay described herein in order for the peptide to be bound and precipitated by the peptide-specific approach, resulting in enrichment of the peptide from the peptide background that is produced during sample digestion. Without wishing to be bound by theory, the length of the peptide and its composition can influence its m/z value and thus its detectability during MS analysis. For example, the peptide VHPVHAGPIPPGQMR (SEQ ID NO: 14) was detected according to the methods described herein, but VHPVHAGPIPPGQMR (SEQ ID NO: 14) peptide appeared more variable among and within the various HIV-1 subtypes than the two peptides (e.g., (E/D) TINEEAAEWDR (SEQ ID NO: 15) or MYSPTSILDI (R/K) (SEQ ID NO: 16)) elected to pursue. Without wishing to be bound by theory, using the VHPVHAGPIPPGQMR (SEQ ID NO: 14) peptide as a target biomarker would have made it more difficult to identify an antibody that could detect most of the HIV-1 subtypes due to the variability. For example, HIV shows a high mutation on its genome, and the mutations can be identified in the peptide sequence region targeted. Here, to cover all HIV-1 strains, multiple peptide sequences with amino acid variations can be used to achieve the highest coverage. Unless the patient is infected with multiple HIV-1 strains, the combination of these peaks may not be observed.

This disease-specific biomarker as described herein can be indicative of an infection or disease. The disease-specific biomarker can also be used to detect infectious pathogens (such as bacterial pathogens, fungal pathogen, and/or viral pathogens). In embodiments, the disease comprises a bacterial disease or a viral disease. Non-limiting examples of a pathogenic bacterium, of which can cause disease/infection in a mammal (such as humans) include: Streptococcus (e.g., S. pneumoniae, S. sanguinis, S. bovis), Pseudomonas (e.g., P. aeruginosa, P. oryzihabitans), Salmonella (e.g., S. enterica, S. bongori), Shigella (e.g., S. dysenteriae, S. flexneri), Mycoplasma (e.g., M. pneumoniae, M. genitalium), Mycobacterium (e.g., M. tuberculosis, M. leprae, M. kansasii, M. marinum, M. ulcerans). Non-limiting examples of a pathogenic fungus, of which can cause disease/infection in a mammal (such as humans) include: Pneumocystis (e.g., P. carinii, P. jirovecii). For example, M. tuberculosis can cause tuberculosis. For example, nontuberculous mycobacterial (NTM) infection can cause NTM lung disease. Non-limiting examples of a pathogenic virus which can cause disease/infection include Orthomyxoviridae family viruses (e.g. Influenza A and B), Paramyxoviridae family viruses (e.g. respiratory syncytial virus and Parainfluenza viruses 1-4), Filoviridae family viruses (e.g. Bundibugyo virus, Reston virus, Sudan virus, Taï Forest virus, Zaire ebolavirus (Ebola virus), Marburg virus (Marburg hemorrhagic fevers)), Bunyaviridae family viruses (e.g. hantaviruses, Rift Valley fever and Crimean-Congo hemorrhagic fever), Arenaviridae family viruses (e.g. Lassa fever virus), Retroviridae family viruses (e.g., HIV, FIV), and Flaviviridae family viruses (e.g. dengue and yellow fever).

For example, the diseases associated with the above pathogens comprises tuberculosis, Ebola virus disease (EVD), nontuberculous mycobacterial (NTM) lung disease, human immune deficiency virus (HIV) infection (such as AIDS and Kaposi's Sarcoma), or gut microbial perturbations. In an embodiment, the disease is not solely nontuberculous mycobacterial (NTM) lung disease. In an embodiment, the methods of detection and identification of a disease-specific biomarker comprise subjecting the sample to a mass spectrometry-based analytical technique and detecting m/z peaks in the mass spectrum. In an embodiment the method comprises identifying the subject from which the biological sample was obtained as having a disease based on the m/z peaks in the mass spectrum. In one embodiment, the disease-specific biomarker has a molecular weight in the range of about 500 Da to about 5000 Da. For example, the molecular weight comprises less than 500 Da, about 500, about 600 Da, about 700 Da, about 800 Da, about 900 Da, about 1000 Da, about 1500 Da, about 2000 Da, about 2500 Da, about 3000 Da, about 3500 Da, about 4000 Da, about 4500 Da, about 5000 Da, and greater than 5000 Da.

Aspects of the invention are drawn towards contacting an enzyme-digested sample with an antibody-modified solid support (AMSS). For example, an enzyme-digested sample comprises a trypsin digested sample. In some embodiments, the contacting can occur under conditions that promote binding of the AMSS to disease specific antibodies if present in the contacted biological sample. The term antibody-modified solid support (AMSS) refers to any solid support that can be porous or non-porous. In some embodiments, the AMSS can be any solid phase substance for which an antibody can be bound. In further embodiments, the surface of the AMSS can be modified to produce textured surfaces, for example as described in PCT Publication No. WO/2018/151930, which is hereby incorporated by reference in its entirety, for defining features such as pores, ridges and valleys or other similar morphologies. In further embodiments, a non-porous support can be transparent, such as glass, or alternatively, plastic, polystyrene, polyethylene, dextran, polypropylene and the like. In other embodiments, the porous support can be nitrocellulose, porous glass, or agarose (such as sephadex). Non-limiting examples of the AMSS include: a bead (such as plastic, glass, silica, magnetic, agarose, and the like), a nanoparticle (such as a nanodisk), a microparticle (such as a microdisk or a magnetic bead), a film, a rod. In some embodiments, the nanoparticle is about 10 nm to about 1000 nm in size. For example, the nanodisk is about 800 nm. In some embodiments, the nanodisk can be about 50 nm, about 100 nm, about 200 nm, about 300nm, about 400 nm, about 500 nm, about 600 nm, about 700 nm, about 800 nm, about 900 nm, or about 1000 nm. For example, the AMSS comprises a magnetic bead. In some embodiments the magnetic bead is about 1 micron to about 100 microns in size. For example, the magnetic bead is about 1 micron to about 2.8 microns. For example, the magnetic bead can be about 0.5 μm, about 1 μm, about 1.25 μm, about 1.50 μm, about 1.75 μm, about 2 μm, about 2.25 μm, about 2.5 μm, about 2.75 μm, about 3μm, about 3.25 μm, about 3.50 μm, about 3.75 μm, about 4 μm, about 4.25 μm, about 4.5 μm, about 4.75 μm, about 5μm, about 5.25 μm, about 5.50 μm, about 5.75 μm, about 6 μm, about 6.5 μm, about 7 μm, about 7.5 μm, about 8 μm, about 8.5 μm, about 9 μm, about 9.5 μm, about 10 μm, about 11 μm, about 12 μm, about 13 μm, about 14 μm, about 15 μm, about 16 μm, about 17 μm, about 18 μm, about 19 μm, about 20 μm, about 25 μm, about 30 μm, about 35 μm, about 40 μm, about 45 μm, about 50 μm, about 55 μm, about 60 μm, about 65 μm, about 70 μm, about 75 μm, about 80 μm, about 85 μm, about 90 μm, about 95 μm, or about 100 μm. In some embodiments, the AMSS can be etched for structure or made porous. For example, etching or increasing porosity can increase surface area giving more area for antibody attachment and/or decreasing unwanted binding. Other non-limiting examples include the matrix in a chromatographic column, a filter, or a surface. Without wishing to be bound by theory, the AMSS can be conjugated with an antibody directed to a target antigen, which serves as a marker for the desired disease or infection.

The method further comprises sensing the disease-specific biomarker in a concentration comprising the range of less than 0.1 pM, about 0.1 pm, about 0.2 pM, about 0.3 pM, about 0.4 pM, about 0.5 pm, about 0.6 pM, about 0.7 pM, about 0.8 pM, about 0.9 pM, about 1.0 pM, about 2.0 pM, about 5 pM, about 10 pM, about 20 pM, about 30 pM, about 50 pM, bout 60 pM, about 70 pM, about 80 pM, about 90 pM, about 0.1 nM, about 0.2 nM, about 0.3 nM, about 0.4 nM, about 0.5 nM, about 0.6 nM, about 0.7 nM, about 0.8 nM, about 0.9 nM, about 1.0 nM, about 2.0 nM, about 5 nM, about 10 nM, about 20 nM, about 30 nM, about 50 nM, about 60 nM, about 70 nM, about 80 nM, about 0.1 μM, about 0.2 μM, about 0.3 μM, about 0.4 μM, about 0.5 μM, about 0.6 μM, about 0.7 μM, about 0.8 μM, about 0.9 μM, about 1.0 μM, about 2.0 μM, about 3.0 μM, about 4.0 μM, about 5.0 μM, about 10 μM, about 20 μM, about 30 μM, about 50 μM, about 60 μM, about 70 μM, about 80 μM, about 0.1 mM, about 0.2 mM, about 0.3 mM, about 0.4 mM, about 0.5 mM, about 0.6 mM, about 0.7 mM, about 0.8 mM, about 0.9 mM, about 1.0 mM, or greater than 1.0 mM. For example, in tuberculosis patient blood, the disease specific biomarker is in a concentration of about 0.1pm to about 200 pM. For example, the Ebola disease-specific biomarker VP40 is in a concentration of about 1 μM to about 6 μM.

In an embodiment, the disease-specific biomarker concentration range will vary depending upon the disease application. Without wishing to be bound by theory, the approach is designed to work with low abundance biomarkers. For example, the biomarkers comprise protein biomarkers in blood. In another embodiment, the method is drawn towards measuring the absolute concentration of the biomarker in a sample relative to a spiked-in internal standard peptide. Without wishing to be bound by theory, the pre-treatment biomarker can be used to evaluate the relative severity of the infection. For example, the difference between biomarker levels at time points before and after treatment initiation can be useful as a measure of disease progression or response to treatment.

Aspects of the invention are drawn towards methods of detecting or identifying a disease-specific biomarker by subjecting a biological sample to a mass spectrometry-based analytical technique. In an embodiment, the mass spectrometry technique comprises at least one hard ionization technique or at least one soft ionization technique. The phrase “soft ionization technique” refers to an ionization technique that produces little to no fragmention ions. For example, soft ionization techniques can comprise matrix-assisted laser desorption/ionization (MALDI), electrospray ionization (ESI), fast atom bombardment (FAB), chemical ionization (CI), atmospheric-pressure chemical ionization (APCI), desorption electrospray ionization (DESI), atmospheric pressure photoionization (APPI), or secondary ion mass spectrometry (SIMS). The pharse “hard ionization technique” refers to an ionization technique that produces ions with greater fragmentation compared to soft ionization techniques. For example, the hard ionization technique comprises electronic ionization (EI). In an embodiment, the mass spectrometry-based analytical technique comprises selected reaction monitoring (SRM), parallel reaction monitoring (PRM), scheduled parallel reaction monitoring (sPRM), multiple reaction monitoring (MRM), scheduled multiple reaction monitoring (SMRM), immunoprecipitation scheduled parallel reaction monitoring (iSPRM), or nanoelectrospray-tandem mass spectrometry (NanoES-MS/MS).

In another embodiment, the mass spectrometry-based analytical technique comprises tandem mass spectrometry (MS²). The term “tandem mass spectrometry” refers to a mass spectrometry based analytical technique where an analyte is ionized to generate ions which are then separated by a mass analyzer and subsequently undergo further fragmentation and are subjected to at least one other mass analyzer before detection. Herein, the term “tandem mass spectrometry” can also refer to multi-stage or sequential mass spectrometry (MSⁿ), where n is a number greater than 2 and n-1 is the generation of product ion spectra. The term “product ion” refers to an ion that is the product of a fragmentation of a parent ion. For example, the tandem mass spectrometry (MS²) technique comprises selected reaction monitoring (SRM), parallel reaction monitoring (PRM), scheduled parallel reaction monitoring (sPRM), multiple reaction monitoring (MRM), scheduled multiple reaction monitoring (sMRM), immunoprecipitation scheduled parallel reaction monitoring (iSPRM), or nanoelectrospray-tandem mass spectrometry (NanoES-MS/MS). Additionally, the MS² technique can comprise data-independent acquisition (DIA) or data-dependent acquisition (DDA). For example, MSⁿ can be MS², MS³, MS⁴, MS⁵, or sequential generations of the target ions. For example, to conduct a relative quantification of markers between two samples, a labeling tag such as TMT will be applied, and MS³ is used to generate quantitative results.

In an embodiment, tandem mass spectrometry (MS²) comprises a technique to induce fragmentation comprises collision-induced dissociation (CID), electron capture dissociation (ECD), electron transfer dissociation (ETD), negative electron transfer dissociation (NETD), electron-detachment dissociation (EDD), charge transfer dissociation (CTD), photodissociation, infrared multiphoton dissociation (IRMPD), blackbody infrared radiative dissociation (BIRD), or surface induced dissociation (SID).

In addition to an ionization technique, the mass spectrometry technique utilizes at least one mass analyzer comprising a quadrupole mass analyzer, a time of flight (TOF) mass analyzer, a magnetic sector mass analyzer, an electrostatic sector mass analyzer, a quadrupole ion trap mass analyzer, an orbitrap mass analyzer, or ion cyclotron resonance mass analyzer.

In a further embodiment, the invention is drawn towards a mass spectrometry-based analytical technique applied to the disease-specific biomarker bound to the AMSS. In another embodiment, the invention is drawn towards a mass spectrometry-based analytical technique applied to an eluted disease-specific biomarker.

In some embodiments, the methods use MALDI-TOF-MS based methods, but in other embodiments, other mass spectrometry techniques can be applied by one skilled in the art. In MALDI (matrix assisted laser desorption ionization) TOF (time-of-flight) mass spectrometry, a sample/matrix mixture is placed on a defined location (“spot”, or “sample spot” herein) on a metal plate, known as a MALDI plate. A laser beam is directed onto a location on the spot for a very brief instant, causing desorption and ionization of molecules or other components of the sample. The sample components “fly” to an ion detector. The instrument measures mass to charge ratio (m/z) and relative intensity of the components (molecules) in the sample in the form of a mass spectrum. The term mass to charge ratio (m/z) refers to the ratio of the mass of the species to the charge state of the species. Described herein, it is understood that a m/z peak refers to the [M+H]⁺ ion. It is understood by one skilled in the art that the hydrogen ion can be replaced with another ion. For example, the ion can be a cation or an anion. Non-limiting examples of cations include sodium (Na⁺), potassium (K⁺), and cesium (Cs⁺). It is also understood by one skilled in the art that the nominal or exact mass of the analyte will not change, but the m/z will change based upon the counter ion used. For example, the m/z for an [M+H]⁺ peak will be different than the m/z for [M+Na]⁺ peak, while the mass of M remains the same. An m/z peak can also refer to multiply charged species. As described herein the presence of particular disease-associated target antigens in the sample provides infection information, for example for tuberculosis (TB) diagnosis and for monitoring TB therapy.

In some embodiments, the method uses the mass spectromety-based analytical technique combined with a separation technique. In some embodiments, the mass spectrometry-based analytical technique combined with a separation technique comprises LC-MS based methods or gas chromatography-mass spectrometry (GC-MS). For example, the mass-spectrometry-based analytical technique comprises column chromatography with liquid-solid interaction (forward and/or reverse phase interactions) coupled to a mass spectrometry technique. Liquid chromatography (LC) coupled mass spectrometry (MS) is an accurate analytical method for biomarker analysis when used with targeted MS approaches multiple reaction monitoring (MRM), selective reaction monitoring (SRM) and parallel reaction monitoring (PRM) modes. In LC-MS, target protein identification is enhanced by detection of target peptides with characteristic LC column elution times and charge-to-mass ratios. Multiple biomarker-derived peptides can be analyzed to provide improved detection specificity and the precursor ions of these peptides can also be fragmented in the MS collision cell to allow direct determination of their amino acid sequences. In some embodiments, the method uses LC-MRM-MS based methods. In some embodiments, the method uses LC-iSPRM-MS based methods. In some embodiments, the LC-MS based method comprises liquid chromatography mass spectrometry (LC-MS), liquid chromatography with tandem mass spectrometry (LC-MS-MS or LC-MS²), liquid chromatography scheduled parallel reaction monitoring mass spectrometry (LC-sPRM-MS), or immunoprecipitation coupled liquid chromatography scheduled parallel reaction monitoring mass spectrometry (LC-iSPRM-MS). In some embodiments, the method uses immunoprecipitation (IP) with nanoelectrospray-tandem MS (NanoES-MS/MS).

Accordingly, in another aspect, provided herein is a method of detecting and identifying pathogen-associated peptides in a sample. In some embodiments, MALDI-TOF-MS-based detection methods where specific Mycobacterium-associated peptides are detected and identified in enzyme-digested human serum or plasma samples using antibody-modified iron oxide nanoparticles. Unlike conventional uses of magnetic dynabeads, where the enriched and bound target peptides are eluted out after enrichment, the present methods include spotting antibody-modified NPs directly onto a MALDI plate after capturing target peptide if present in the sample. In this manner, the process is simplified and has a lower detection limit. By combining different peptides digested from Mycobacterium proteins, infection caused by Mycobacterium tuberculosis can be distinguished from M. kansasii and other mycobacteria. As described herein, the methods provide a new tool for diagnosing a subject as having a particular Mycobacterium infection or co-infection with two or more mycobacteria species.

In some cases, the method comprises contacting a trypsin-digested biological sample with antibody-modified magnetic nanoparticles (NPs) under conditions that promote binding of the antibody-modified magnetic NPs to its target if present in the contacted biological sample, where the antibodies bind specifically to a disease-specific target antigen; spotting the contacted antibody-modified magnetic NPs to a MALDI plate; performing MALDI-TOF-MS analysis of the spotted NPs, whereby a mass spectrum of a molecule is obtained; and detecting m/z [M+H]⁺ peaks in the mass spectrum, where a m/z peak at 1594 is indicative of a disease-specific target antigen associated with co-infection by Mycobacterium tuberculosis complex subspecies and one or more of M. kansasii, M. marinum, and M. ulcerans infection, where a m/z peak at 1901 or 2004 is indicative of a disease-specific target antigen associated with infection by Mycobacterium tuberculosis complex subspecies, and where a m/z peak at 2032 is indicative of a disease-specific target antigen associated with infection by M. kansasii. This mass target can also be observed from M. gordonae, which is another NTM species causing human infection.

The magnetic nanoparticles can be Fe₃O₄, MnFe₂O₄, CoFe₂O₄, NiFe₂O₄, or ZnFe₂O₄. In some cases, the antibody-modified magnetic nanoparticles are prepared by the steps of: surface functionalizing magnetic nanoparticles by contacting (3-Glycidyloxypropyl) trimethoxysilane (GLYMO) to a monodispersed preparation of magnetic nanoparticles; and conjugating antibodies that bind specifically to the disease-specific target antigen to the GLYMO contacted magnetic nanoparticles.

In some cases, wherein, prior to conjugating antibodies, the GLYMO contacted magnetic nanoparticles are contacted to a blocking solution selected from Tris-NaCl buffer (pH>8.0), (2-aminoethyl) ethanol, or NH₂(CH₂CH₂O) nCH₃; where n is a number from 1 to 100.

In some embodiments, Mycobacterium-specific peptides are peptides detected following trypsin digestion of mycobacterial proteins such as 10-KDa culture filtrate proteins (CFP-10), which are early secretory antigens. In some cases, the disease-specific target antigen has a molecular weight in the range of about 500 Daltons (Da) to about 5000 Daltons (Da). In particular, the disease-associated antigens include two segments of CFP-10 detected following trypsin digestion: peptide 1594 (TDAATLAQEAGNFER, MW: 1593.75; SEQ ID NO:1) and peptide 2004 (TQIDQVESTAGSLQGQWR, MW: 2003.98; SEQ ID NO:2). Peptide 2004 is only found in Mycobacterium tuberculosis complex subspecies (M. Tb, M. africanum, M. bovis, M. canettii, M. caprae, M. orysis, M. pinnipedii); while peptide 1594 is found in Mycobacterium tuberculosis complex subspecies (M. Tb) as well as M. kansasii, M. marinum, M. ulcerans, and or M. gastrii. Accordingly, by detecting the presence or absence of particular peptides in a trypsin-digested patient serum or plasma sample, it is possible to distinguish between infection by Mycobacterium tuberculosis complex subspecies and by M. kansasii, M. marinum, M. ulcerans, and or M. gastrii. See Table 1.

TABLE 1
Diagnosis of infection based on the detection of peptide sequence after serum digestion

| Peptide 1594 | Peptide 1901 | Peptide 2004 | Peptide 2032 | Diagnosis |
|---|---|---|---|---|
| + | − | + | − | Co-infection with M. Tb and |
| | | | | M. kansasii, M. marinum, |
| | | | | M. ulcerans, and/or M. gastrii |
| − | + | + | − | Infected with M. Tb complexes |
| − | − | − | + | Infected with M. kansasii, |
| | | | | M. marinum, M. ulcerans, |
| | | | | and/or M. gastrii. |
| − | − | − | − | No infection with M. Tb |
| | | | | complexes or M. kansasii, |
| | | | | M. marinum, M. ulcerans, |
| | | | | and/or M. gastrii |

A m/z [M+H]⁺ peak at 1594 is indicative of a disease-specific target antigen associated with co-infection by Mycobacterium tuberculosis complex subspecies and one or more of M. kansasii, M. marinum, and M. ulcerans infection. A m/z [M+H]⁺ peak at 1901 or 2004 is indicative of a disease-specific target antigen associated with infection by Mycobacterium tuberculosis complex subspecies. A m/z [M+H]⁺ peak at 2032 is indicative of a disease-specific target antigen associated with infection by M. kansasii and/or M. gastrii.

TABLE 12
The VP40 peptide targets for five Ebola species identification

| | SEQ | | | | | | |
|---|---|---|---|---|---|---|---|
| Peptide Sequence | ID NO: | M + H⁺ | EBOV | SUDV | BDBV | TAFV | RESTV |
| LGPGIPDHPLR* | 17 | 1171.7 | + | | + | + | + |
| LGQGIPDHPLR | 18 | 1202.7 | | + | | | |
| LRPILLPNK | 19 | 1063.7 | + | | | | |
| LRPILLPGK* | 20 | 1006.7 | | | + | | + |
| LRPILLPGR | 21 | 1034.7 | | | | + | |
| LRPVLLPGK | 22 | 992.7 | | + | | | |

Table 12 shows a m/z [M+H]⁺ peak at 1171.7 is indicative of a disease-specific peptide sequence LGPGIPDHPLR* (SEQ ID NO: 17) which indicates infection with Ebola virus (EBOV), Budibugyo virus (BDBV), Taï Forest virus (TAFV), and/or Reston virus (RESTV). A m/z [M+H]⁺ peak at 1202.7 is indicative of the disease specific peptide sequence LGQGIPDHPLR (SEQ ID NO: 18) which indicates infection with Sudan virus (SUDV). A m/z [M+H]⁺ peak at 1063.7 is indicative of the disease specific peptide sequence LRPILLPNK (SEQ ID NO: 19) which indicates infection with Ebola virus (EBOV). A m/z [M+H]⁺ peak at 1006.7 is indicative of the disease specific peptide sequence LRPILLPGK* (SEQ ID NO: 20) which indicates infection with Bundibugyo virus (BDBV) and/or Reston virus (RESTV). A m/z [M+H]⁺ peak at 1034.7 is indicative of the disease-specific peptide LRPILLPGR (SEQ ID NO: 21) which indicates infection with Taï Forest virus (TAFV). A m/z [M+H]⁺ peak at 992.7 is indicative of the disease-specific peptide sequence LRPVLLPGK (SEQ ID NO: 22) which indicates infection with Sudan virus (SUDV).

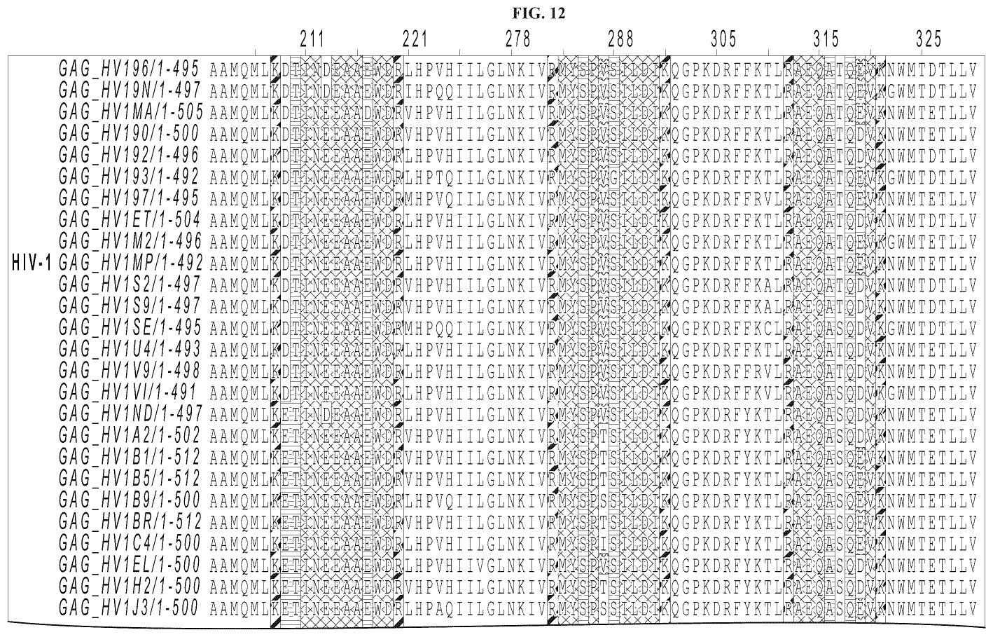

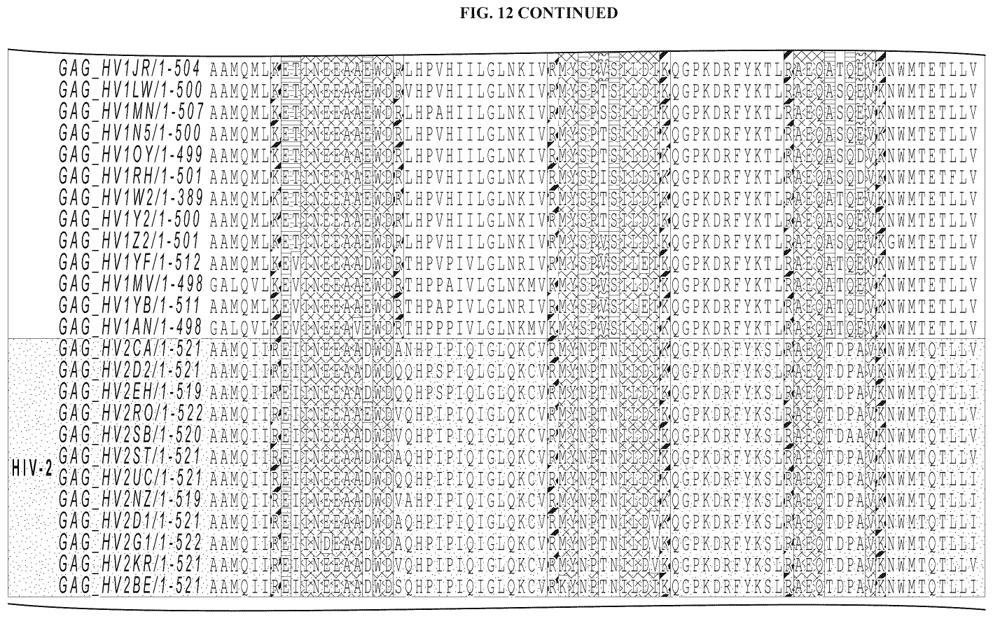

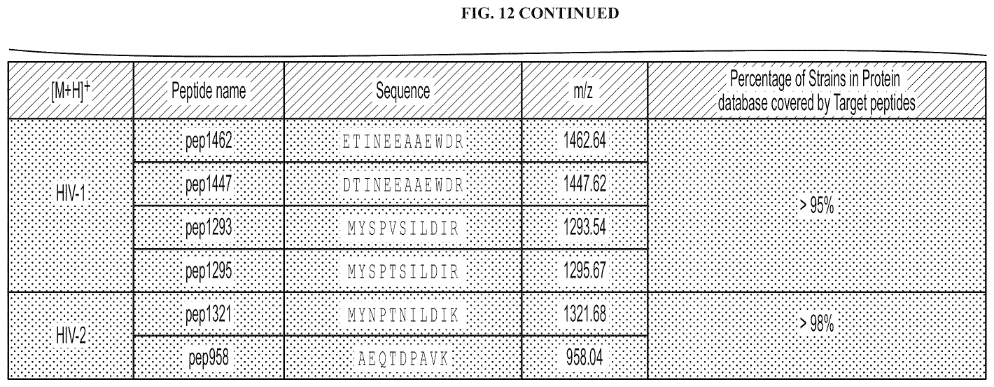

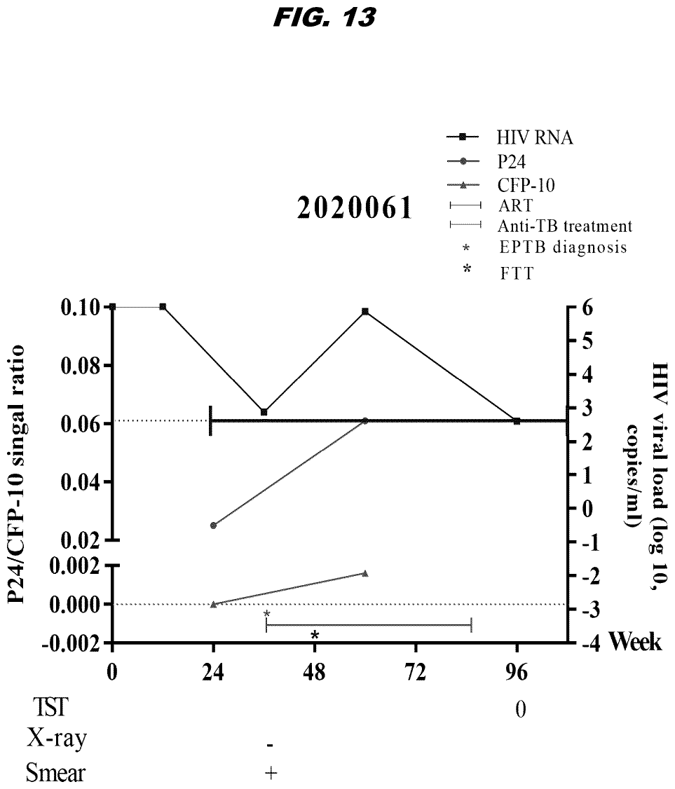

In some embodiments, HIV-1/2 specific peptides are peptides detected following trypsin digestion. For example, HIV-1 specific peptides include ETINEEAAEWDR (SEQ ID NO: 5) (m/z peak at 1462.64); DTINEEAAEWDR (SEQ ID NO: 6) (m/z peak at 1447.62); MYSPVSILDIR (SEQ ID NO: 9) (m/z peak at 1293.54); and MYSPTSILDIR (SEQ ID NO: 7) (m/z peak at 1295.54), which collectively cover 95% of HIV-1 stains. HIV-2 specific peptides include MYNPTNILDIK (SEQ ID NO: 23) (m/z peak at 1321.68), MYNPPTNILDIK (SEQ ID NO: 12) (m/z peak at 1321.68), and AEQTDPAVK (SEQ ID NO: 11) (m/z peak at 958.04) which collectively cover 98% of HIV-2 strains. See FIG. 12. For example, with HIV-2, multiple strains are covered. Without wishing to be bound by theory, more than one sequence is targeted here due to the natural mutation.

In some cases, the trypin-digested biological sample comprises one or more internal reference standards. In an embodiment, the internal reference standard comprises an isotopically labed sample. In one embodiment, the sample is an synthetic peptide that has the same sequence as the disease-specific biomarker, but is labeled with a stable heavy isotope to shift its m/z ratio by a fixed amount. For example, the isotopically labeled synthetic peptide is spiked into the sample in a known amount so that its signal can be compared to the biomarker target signal to allow the absolute quantification of an unknown amount of biomarker present in a sample.

In some cases, the method further comprises generating a reference mass spectrum in which a peak corresponding to one or more microorganism antigens of interest is reliably present in the reference mass spectrum.

In some embodiments, the method used to detect serum/plasma biomarkers is integration of immunoprecipitation (IP) with nanoelectrospray-tandem MS (NanoES-MS/MS). This approach is used to detect the circulating level of a virulence factor secreted by Mycobacterium tuberculosis (Mtb), the 10 kDa culture filtrate protein (CFP-10) described above. Before one can perform such targeted MS analyses, patient serum/plasma samples need to be digested to liberate target peptides from their respective biomarkers. This process is complicated by the high abundance, diversity and dynamic range of the proteins present in serum. To address this issue, a sample denaturation and protein chromatography protocol is used to reduce the abundance of off-target proteins in these samples, after which the protein depleted samples are trypsin digested and subjected to IP without a buffer exchange step. This process reduces the amount of trypsin required to efficiently and reproducibly generate target peptides used for biomarker identification and quantitation. This process has several advantages over standard approaches, as it: 1) avoids sample dilution and can be scaled as-needed to account for the observed concentration of the target biomarker in the sample; 2) permits efficient liberation of trace amount of target peptide even at low enzyme-to-substrate mass ratios (i.e., 1:500-1:1,000); and 3) does not require the time-consuming reduction and alkylation of disulfide bonds, as many pathogen-derived biomarkers do not contain these features. This workflow allows a target biomarker to be detected down to sub-pM lower limits of detection and allows multiplex enrichment and detection of target peptides from one or more biomarkers of interest.

In some embodiments, the method used to detect serum/plasmid biomarkers is LC-PRM-MS.

As used herein, the term “sample” means non-biological samples and biological sample and encompasses clinical specimens (diagnostic samples collected as part of standard clinical procedures). Non-biological samples include those prepared in vitro comprising varying concentrations of a target molecule of interest in solution. Biological samples include, without limitation, whole blood, lymph, serum, plasma, urine, saliva, sputum, breath extract (meaning exhaled air captured in a solution), bone marrow, aspirates (nasal, lung, bronchial, tracheal), eye fluid, amniotic fluid, feces other bodily fluids and secretions, cells, and tissue specimens and dilutions of them. Any suitable biological sample (“biosample”) can be used. For example, a biological sample can be a specimen obtained from a subject or can be derived from such a subject. A subject can provide a plurality of biological samples, including a solid biological sample, from for example, a biopsy or a tissue. In some cases, a sample can be a tissue section or cells that are placed in or adapted to tissue culture. A biological sample also can be a biological fluid such as urine, blood, plasma, serum, saliva, tears, cerebrospinal fluid, semen, bile, lymph, or mucus, or such a sample absorbed onto a paper or polymer substrate. A biological sample can be further fractionated, if desired, to a fraction containing particular cell types. In some embodiments, a sample can be a combination of samples from a subject (e.g., a combination of a tissue and fluid sample). In some cases, serum or plasma is obtained from the subject using techniques known in the art. For example, the sample is a liquid biopsy sample.

By “subject” or “individual” or “animal” or “patient” or “mammal,” is meant any subject, for example a mammalian subject, for whom diagnosis, prognosis, or therapy is desired. Mammalian subjects include humans, domestic animals, farm animals, and zoo, sport, or pet animals such as dogs, cats, guinea pigs, rabbits, rats, mice, horses, cattle, cows, and so on. Thus, in addition to being useful for human diagnostic and prognostic applications (e.g., diagnosing a disease in a human patient), the methods and devices of the present invention may also be useful for veterinary treatment of mammals, including companion animals.

In another aspect, provided herein is an article of manufacture such as a kit comprising materials and reagents that can be used to determine whether a subject has a Mycobacterium infection and to identify the Mycobacterium species. An article of manufacture can include, for example, one or more of antibody-modified solid supports (such as magnetic nanoparticles), antibodies, a MALDI-TOF matrix, and sampling materials (e.g., swabs, elution buffer). The article of manufacture can also include instructions for use in practicing a method for detecting and identifying a target antigen associated with infection by one or more Mycobacterium species or virus as provided herein.

In some cases, the kit comprises antibody-modified magnetic nanoparticles (NPs), wherein the antibodies bind specifically to a Mycobacterium or viral peptide. The Mycobacterium or viral peptide can have a sequence comprising TDAATLAQEAGNFER (SEQ ID NO:1), TQIDQVESTAGSLQGQWR (SEQ ID NO:2), WDATATELNNALQNLAR (SEQ ID NO:3), or TQIDQVESTAASLQAQWR (SEQ ID NO:4).

The kits described herein also can optionally include instructions for treating a patient based on the presence or absence of particular mycobacterial antigens in the patient's sample as described herein.

The terms “determining”, “measuring”, “evaluating”, “assessing,” “assaying,” and “analyzing” can be used interchangeably herein to refer to any form of measurement and include determining if an element is present or not. These terms can include both quantitative and/or qualitative determinations. Assessing may be relative or absolute. “Assessing the presence of” includes determining the amount of something present, as well as determining whether it is present or absent.

In yet other embodiments, the instructions are not themselves present in the kit, but means for obtaining the instructions from a remote source, e.g., via the Internet, are provided. An example of this embodiment is a kit that includes a web address where the instructions can be viewed and/or from which the instructions can be downloaded. Conversely, means may be provided for obtaining the subject programming from a remote source, such as by providing a web address. Still further, the kit may be one in which both the instructions and software are obtained or downloaded from a remote source, as in the Internet or World Wide Web. Some form of access security or identification protocol may be used to limit access to those entitled to use the subject invention. As with the instructions, the means for obtaining the instructions and/or programming is generally recorded on a suitable recording medium.

### EXAMPLES

Examples are provided below to facilitate a more complete understanding of the invention. The following examples illustrate the exemplary modes of making and practicing the invention. However, the scope of the invention is not limited to specific embodiments disclosed in these Examples, which are for purposes of illustration only, since alternative methods can be utilized to obtain similar results.

### Example 1: Identifying Target Antigens Associated With Mycobacterium infection

### Selection of Tryptic Digested Peptides Segment From CFP-10

10-KDa culture filtrate proteins (CFP-10) are a series early secretory antigens from mycobacteria. Two CFP-10 segments from CFP-10 resulting from trypsin digestion (peptide 1594: TDAATLAQEAGNFER (SEQ ID NO: 1), MW: 1593.75, and peptide 2004: TQIDQVESTAGSLQGQWR (SEQ ID NO: 2), MW: 2003.98) were selected as targets. Peptide 2004 is found only in Mycobacterium tuberculosis complex subspecies (MTb, M. africanum, M. bovis, M. canettii, M. caprae, M. orysis, M. Pinnipedii, etc); while peptide 1594 can be found in CFP-10 secreted from M. kansasii, M. marinum, M. ulcerans, etc, as well as the Mycobacterium tuberculosis complexes. Therefore, by detecting presence or absence of two peptides in patient serum/plasma, we can distinguish the infection of Mycobacterium tuberculosis from infection of M. kansasii or M. marinum, and thus provide reasonable information for physicians to medically treat patients.

Synthesis of Monodispersed Iron Oxide Fe₃O₄ NPs

Monodispersed iron oxide nanoparticles (Fe₃O₄) were synthesized by solvothermal method using FeCl_{3 7}H₂O in ethylene glycol. After separation using a NdFeB magnet, the NPs were washed with ethanol.

After drying the washed NPs in vacuum overnight, the iron oxide NPs were ultrasonically washed with diluted HCl acid, followed by ethanol washing two additional times following by drying overnight under a vacuum.

### Bioconjugation of Superparamagnetic Nanoparticles With Anti-1593 Antibody

10 mg of iron oxide NPs were dispersed in 800 uL of PBS buffer (pH: 8.3, adjusted by using 1.0 M NaOH), followed by adding in anti-1593 antibody, with an antibody/iron oxide ratio of 7 μg/mg. The mixture were incubated at room temperature for 3 hours. The antibody modified NPs were separated by DynaMag2. The NPs were dispersed in 1 mL of Tris-NaCl buffer (Tris: 0.2 M; NaCl 0.1 M; pH: 8.0) which is used to block excess epoxy group on the NPs surface. The mixture was incubated on Hula mixer for another 30 minutes (min.). The antibody modified NPs were separated by Dynamag2 and washed using deionized (DI) water trice. The final antibody modified NPs were dispersed in 500 μL DI water, and stored at −4 C for further use.

### Bioconjugation of Superparamagnetic Nanoparticles With Anti-2004 Antibody

10 mg of iron oxide NPs were dispersed in 800 uL of PBS buffer (pH: 8.3, adjusted by using 1.0 M NaOH), followed by adding in anti-2004 antibody, with an antibody/iron oxide ratio of 7 μg/mg. The mixture were incubated at room temperature for 3 hours. The antibody modified NPs were separated by DynaMag2. The NPs were dispersed in 1 mL of Tris-NaCl buffer (Tris: 0.2 M; NaCl 0.1 M; pH: 8.0) which is used to block excess epoxy groups on the NPs surface. The mixture was incubated on Hula mixer for another 30 min. The antibody-modified NPs were separated by Dynamag2 and washed using deionized water trice. The final antibody-modified NPs were dispersed in 500 μL DI water, and stored at −4° C. for further use.

### Tryptic Digestion of Patient's Serum Plasma

To 100 μL of serum/plasma were added 400 μL of NH₄HCO₃ (100 mM), followed by 10 uL of trypsin (1 μg/μl). The mixture was digested at 20% microwave power for 5 min in water bath, the water was changed then repeat the microwave digestion for total 6 times. The mixture was incubated in a Hula mixer overnight at 37° C., and then centrifuged at 10,000 rcf for 5 min. The top clear supernatant was transferred to a new EP tube.

### Capture of Peptide 1594 From CFP-10

Isotopic peptide 1603 (50 nM, 2 μL, total 100 fmol) was added into a trypsin digested plasma/serum sample. The sample was then vortexed for about 10 seconds to mix the sample. Five microliters of antibody-modified NPs were added to the mixture. The mixture was then incubated with Hula mixer for 3 hours at room temperature. The mixture was centrifuged for less than 10 seconds to retain liquid to the bottom of the tube. A magnet (or DynaMag 2) was used to separate supernatant from antibody-modified NPs. The NPs were washed 3 times in a washing buffer (50 mM Tris HCl, 150 mM NaCl, 0.2% triton x-100, pH: 7.4˜7.5, approximately 80 μL/tube). Before the supernatant was removed, the tube was centrifuged for fewer than 10 seconds to keep liquid down to the tube bottom. 100 μL of DI water was used to wash the NPs. Wash the NPs until no bubbles are visible in the solution. The tube of washed NPs was centrifuged and NPs were separated with a magnet. Washed NPs were dispered in about 4 μL of DI water, and then spotted onto a MS 96-well MALDI plate (2 μL per spot). Onto each spot, α-Cyano-4-hydroxycinnamic acid (CHCA) (6 mg/mL in CH₃CN/H₂O=6/4 (v/v) in 0.1% TFA; 0.7 μL per spot) was spotted.

### Capture of Peptide 1594 From CFP-10

Repeat procedure in example 5, in which isotopic peptide 2014 were used to replace isotopic peptide 1604.

### Example 2: Immunoprecipitation and Targeted MS Detection of Proteolytic Peptides Dervied From Human Pathogen in Serum/Plasma

MS analyses have shown promise for improved understanding and diagnosis of several important human diseases, including tuberculosis, Alzheimer's, and multiple cancers, to name just a few. MS offers greater potential for the specific detection of a target biomarker than enzyme linked immunoassays (ELISAs), a common approach for clinical biomarker analysis. ELISA relies on the ability of two assay antibodies to recognize two distinct epitopes on a biomarker of interest. ELISA specificity is determined by the restriction of these epitopes to the target protein and by the fact that both regions must be recognized on the same protein to produce a positive signal.

LC-MS analyses can demonstrate greater specificity for a biomarker target, since they identify a target protein by the detection of target peptides with characteristic LC column elution times and charge-to-mass ratios. Multiple biomarker-derived peptides can be analyzed to provide improved detection specificity and the precursor ions of these peptides can also be fragmented in the MS collision cell to allow direct determination of their amino acid sequence. The SISCAPA (Stable Isotope Standards and Capture by Anti-Peptide Antibodies) approach allows immunoprecipitation (IP) of proteolytic peptides of interest using target peptide antibodies prior to MS analysis of selected MRM transitions.

At least five factors need to be considered in developing a specific IP-MS workflow targeting a pathogen-derived peptide. These are 1) the affinity and specificity of the antibody to the target peptide, 2) the selection criteria for the diagnostic pathogen-derived protein and its signature peptides, 3) the abundance of the protein of interest in the circulation and its limit of detection in the assay, 4) the ability to efficiently separate the target peptide from interfering factors, and 5) the MS signal quality of the selected peptide. Most IP-based assays focus on the first four factors.

SISCAPA employs an addition-only trypsin digestion approach capable of automation, employs a wash step with 3-((3-cholamidopropyl) dimethylammonio)-1-propanesulfonate (CHAPS) buffer to reduce nonspecific binding events, and optimizes the transition for each targeted peptide. Even with these efforts, low abundance peptide species are frequently under-sampled compared to high abundance peptide species and cannot be identified easily by the MS detector. More effort is still required to improve MS assay workflows to overcome this bias.

To address this issue, a new protocol was to produce and isolate target peptides from pathogen-derived proteins present in serum or plasma samples. This method employs a heat-and detergent-based denaturing step suitable for use with a wide range of sample volumes, and which allows a subsequent addition-only trypsin digestion procedure for the generation of target peptides.

This denaturation process involves a simple addition and mixing step with a solution containing phosphate buffered saline (PBS), triton X-100 and sodium dodecyl sulphate (SDS) to achieve a define final concentration, followed by a 5-minute heating step. This procedure has previously been employed to dissociate protein complexes, including antigen-antibody complexes generated as part of the normal immune response to a pathogen, prior to ELISA. This streamlined process also efficiently denatures sample proteins to enhance their efficient cleavage during a subsequent trypsin digestion step. Notably, this procedure does not use reducing and alkylation reagents employed in most proteomic sample processing workflows, and which can interfere with trypsin activity during a subsequent digestion step required to release target peptides for MS analysis. Eliminating this step may improve assay sensitivity if the target protein does not contain one or more disulfide bond, as is the case for many pathogen-derived biomarkers. Detergents present in the denaturing buffer can also suppress non-specific binding during the antibody capture step.

The process used to conjugate an affinity molecule (e.g. a biomarker peptide-specific antibody) to an enrichment matrix is another significant consideration for methods based on affinity capture of target biomarkers. This process can determine the orientation of the affinity molecule on the matrix and thus its ability to interact with its biomarker target. It can also determine the stability of this interaction, which can directly and indirectly influence biomarker detection. Any significant loss of the affinity molecule during the binding step directly decreases the detection sensitivity for the target biomarker as it is not captured on the affinity matrix for subsequent analysis. Release of the affinity molecule during the elution phase, however, can reduce biomarker detection since its binding to the LC column may significantly reduce the binding capacity of the column for the biomarker peptides of interest.

Protein G is used for antibody capture in the SISCAPA workflow, since antibodies captured by interaction with protein G are situated so that both their antigen recognition sites are available for antigen binding. However, this is an affinity interaction, not a covalent interaction, and antibodies can dissociate during both the biomarker binding and elution steps. This is a particular concern during biomarker elution where conditions that disrupt the antigen-antibody complex may also disrupt the antibody-protein G complex, although potential was an issue not described in the SISCPA workflow. Epoxy functional group have also been used to bind biomarker-specific antibodies to an IP matrix. This is a covalent binding and thus there should be very little antibody loss during either the antigen binding or elution procedure, but antibodies are not bound to the affinity matrix in a defined orientation, which may reduce the number of antigen recognition sites available for antigen binding.

We selected protein G magnetic beads as the solid surface for our protocol, since this approach requires less antibody and is faster than the procedure employed to conjugate antibodies to an epoxy-functionalized matrix. However, to prevent the loss of LC column binding capacity through binding of antibody released during the elution of the target peptide, we employed StageTips to bind and antibodies present in the eluate and to capture any large particulates (e.g. capture bead contamination) present in this sample. This clean-up step, which is not part of the SISCAPA workflow, markedly increased the quality of the antibody-associated MS signal, as reflected the reduction in the number and intensity of off-target peaks in the LC spectrum. The ability to rapidly remove particulate contaminants prior to LC analysis is particularly important, since such particles can rapidly obstruct a LC column, leading to a pressure increase that can rapidly destroy it.

MS signal quality relies on the sequence of the target peptides and the parameter settings used for MS data acquisition, which influence MS scanning speed, mass resolution and sensitivity due to the fixed nature of the MS detector. Matrix assisted laser desorption ionization-time of flight MS (MALDI-TOF MS) permits rapid analysis since samples are not first separated on an LC column. MALDI-TOF MS is not sensitive to minor particulate carryover that can destroy LC columns, but is less sensitive than LC-MS and is subject to reduced specificity by contaminating peptides.

LC-based targeted MS workflows offer additional separation of peptides in the upstream of MS analysis, which improves assay specificity. More importantly, optimum MRM transitions of the targeted peptide ion and standardized data processing and interpretation algorithm provide confirmation of its source protein in the analyzed serum sample. We have developed a biomarker specific protocol to optimize the detection of these transitions to improve detection specificity.

By changing the denaturing buffer, addition of a clean-up step, selection of optimum MRM transitions and better interpreting MS signal, this workflow showed an improved sensitivity and specificity in clinical samples

### Sample Pre-Treatment Using A Protein Denaturing Buffer

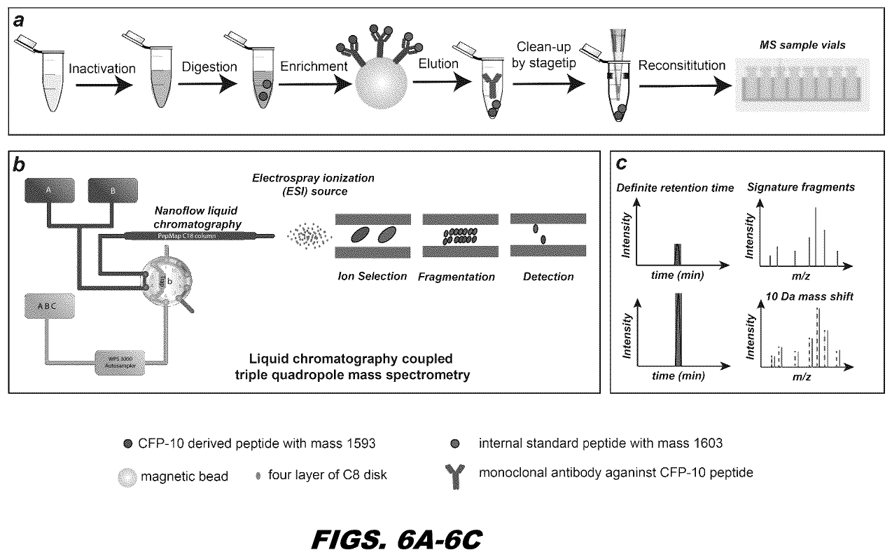

Serum samples are diluted 10× with denaturing buffer, as this was determined to be the optimum dilution for subsequent trypsin digestion and IP. Standard assays utilize 100 μL of serum that is mixed with 900 μL of denaturing buffer in a 1.5 mL of centrifuge tube, which is then sealed and incubated for 5 min in a 100° C. heat block. Caution! When moving the samples from heat block, it is recommended that the assay operator wear thermal insulation gloves. The total volume of serum processed for a sample analysis can be scaled to comply with assay requirements reflecting to serum concentration range of the biomarker of interest. Scaled sample volumes should be processed in 1 mL total aliquots in 1.5 mL centrifuge tubes to ensure consistent heat transfer, which would differ if scaled sample volumes are processed in larger tubes. For example, in an assay for serum detection of the Mtb antigen CFP10, which used 200 μL of serum, two aliquots were prepared for each sample. The schematic present in FIGS. 6A-6C depicts an overview of this workflow.

After this 5 min denaturation step, samples are transferred to a 25° C. Branson 2800 sonicating water bath (2.84 L) and sonicated for 5 minutes at 40 KHz, then transferred to a suitable centrifuge tube rack to stabilize the tubes for subsequent sample processing steps.

After the sonication/cooling step, the tube is pulse-centrifuged (SCILOGEX D1008, 5s at 1,340 g and 25° C.) to spin down any liquid trapped in its cap and 20 μL of IM Tris (hydroxymethyl) aminomethane buffer (pH 10) is added to 1 mL of the denatured serum sample to adjust its pH to ˜8.5. This sample is then vortexed to thoroughly mix its contents and pulse-centrifuged again.

### Protein Digestion

After pH adjustment, denatured serum samples are mixed with 10 μg/mL of sequence grade trypsin, placed on a Hulamixer® and incubated at 20 RPM for 16 hours at 37° C.

After overnight digestion, samples are adjusted to PH ˜7 by the addition of 10 μL of 10% trifluoroacetic acid (TFA), vortexed to clarify the solution of precipitates that may form after adding TFA, and then pulse-centrifuged as above to spin down any liquid present in the tube lid.

### Binding of Peptide-Specific Antibody to Protein G Magnetic Beads

The optimum amounts of magnetic beads and antibody vary among different peptide targets. For the antibody we generated to CFP-10 peptide TDAATLAQEAGNFER (SEQ ID NO: 1), these parameters were determined to be 3 mg of protein G-conjugated magnetic beads and 50 μg of the CFP-10 peptide-specific antibody. Magnetic beads need to be washed with 200 μL of PBS one time to remove any buffer remnant. Beads and antibody are incubated for 1 hour at 25° C. in 400 μL of binding buffer on Hulamixer® set to 20 RPM. After binding, the beads are washed 2× with 200 μL of binding buffer to remove unbound antibody.

Antibody-conjugated beads are suspended in 400 μL of binding buffer and stored at 4° C. for <2 weeks in order to maintain antibody activity.

### Immunoprecipitation (IP) of the Target Peptide

In order to capture CFP-10 peptide TDAATLAQEAGNFER (SEQ ID NO: 1) from serum, 100 μL of trypsin-digested serum is mixed with 0.15 mg of antibody-conjugated magnetic beads at 25° C. on a Hulamixer® set to 20 RPM. This amount of beads/antibody was determined empirically by assessing the MS signal intensity of the target peptide upon analysis of a serum sample spiked with a set amount of CFP-10 protein. The optimum amount of beads/antibody required to analyze another peptide must be determined by this method prior to testing clinical samples.

The beads are combined from two aliquot and suspended in 200 μL of PBS buffer. The beads are transferred into a new centrifuge tube and washed 2× with 200 μL of PBS buffer, and then washed 1× with 100 μL of LC-grade water.

Captured peptides are eluted by incubating the beads with 100 μL of 1% formic acid at 25° C. for 30 minutes on a Hulamixer® set to 20 RPM.

After incubation, microcentrifuge tubes containing the capture beads in elution buffer are transferred to a rare earth magnet and incubated for 20 minutes at 25° C. to allow complete capture of the antigen-depleted beads on the magnet before transfer of the eluate.

### Clean-Up of IP Eluent by Stage Tips

StageTips packed with a C8 disk are conditioned by the addition of 50 μL of 0.1% TFA in acetonitrile followed by 50 μL of 0.1% TFA in water. Added buffer is remove by centrifuging tips placed in adaptor-filled centrifuge tubes for 3 minutes at 2,000 g and 25° C. Caution! Due to the changed height of tubes containing StageTips, the rotor lid cannot be attached during this centrifugation step. Centrifuge tubes must also be placed into the rotor in an ordered fashion so that their will not contact the inner wall of the centrifuge.

After the conditioning process described above, Stage Tips are loaded with the IP eluent and centrifuged using the same conditions as in step 12.

StageTips are then loaded with 50 μL of 0.1% TFA in acetonitrile and centrifuged under as in step 12 to permit completed recovery of the target peptide.

StageTips are removed from these tubes and the StageTip elutate is is evaporated under vacuum, and then reconstituted in 1% formic acid.

After centrifuging the reconstituted samples at 25° C. and 21,000 g for 15 minutes to remove any residual particulates that might escape the StageTip filter, the supernatant is transfer into an MS sample vial. Caution! Prevent contacting the bottom of tube and picking up any invisible particle by leaving one uL of solution.

### MRM Analysis of the Targeted Peptide

To set up the LC and MS methods for a targeted peptide, it is better to prepare a various concentrations of internal standard of that peptide solutions and get the gradient of organic solvent and collision energy optimized based on the MS signal of these solutions. The gradient of organic solvent and MS parameters used here is listed in Document for LC-MRM method.

After loading the sample onto LC column, the MS spectra are collected using the optimum MS method setting.

### MS Data Analysis Using Skyline

MRM data can be processed by a number of available software tools. Based on the design of these tools, they may require the MS/MS spectra of targeted peptide as a reference in order to build a spectral library for peak recognition and evaluation. Here we recommend Skyline as the tool of MRM data processing, due to its compatibility with multiple MS instruments. Skyline needs MS/MS spectrum of targeted CFP-10 peptide to build the library. In this regard, a tryptic digest of Mtb culture filtrates was analyzed by LC-MS/MS and the MS/MS spectra were searched against Mtb protein sequence database through PEAKS Studio. One MS/MS spectrum was identified as the targeted CFP-10 peptide after search and was imported into Skyline. Skyline utilizes it to construct the spectral library.

### Algorithm Development for Data Interpretation

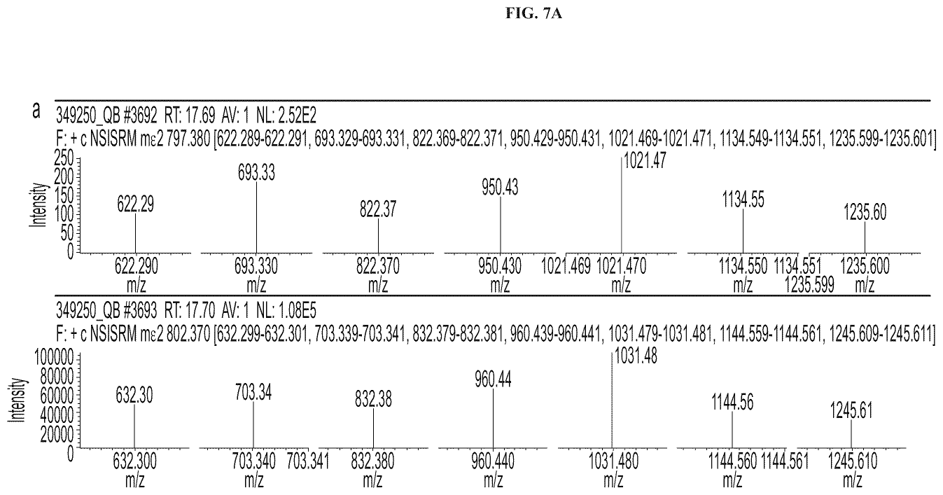

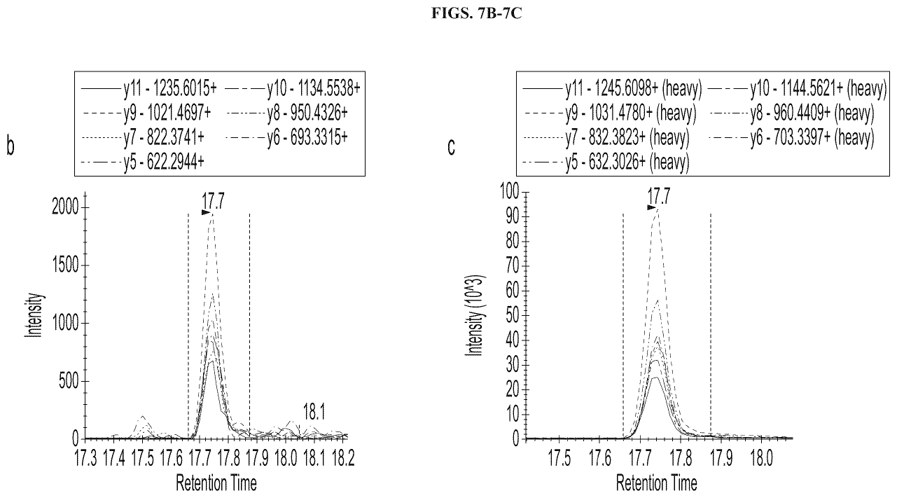

Using the Thermo Xcalibur software, the MRM signal can be reviewed in a transition fashion (FIG. 7a). However, it needs further evaluation of the MS signal quality in order to interpret the result appropriately. When checking the peaks in the ion chromatography, there are numerous peaks which are formed by some transitions (FIG. 7b). The important task is to define the unique peak that belongs to the targeted CFP-10 peptide (light peptide). Since the internal standard (IS) peptide (heavy peptide) was spiked into the sample before IP (FIGS. 6A-6C), we can observe the peak of heavy peptide in a definitive retention time among all samples.

By setting the retention time of heavy IS peptide as a reference, many interfering peaks can be directly filtered out of the MS spectrum. However, some false positive peaks caused by instrumental or chemical noise cannot be filtered out by retention time, since this noise is stochastically distributed across the entire LC gradient.

To evaluate the validity of the target MS signal, it is therefore important to evaluate the relative intensities of each fragment that appears to derive from the target peptide and its matching IS peptide and calculate the similarity between the light (serum) and heavy (IS) peptide fragments. Similar light and heavy peptide signals provide greater confident for the positive detection of the target biomarker signal. The Skyline program returns a dot product (rdotp) to reflect this similarity, where an rdtop value equal to 1 denotes identical signal.

Adjusting the rdotp cutoff produces a corresponding change in the area under the curve (AUC) of the receiver operating characteristic (ROC) curve, which denotes the ability to distinguish positive from false-positive signals. However, the accuracy of this analysis heavily depends on the accuracy of the reference method, which is not well established in tuberculosis diagnosis. To discriminate possible Mtb-infected patients from subjects without TB, more parameters are needed to interpret the MS signal. The number of observed fragments from a target peptide, and their intensities, are useful indicators.

The baseline of the background noise in an MS spectrum must be subtracted from target peaks to determine if these peaks represent artifacts or real signal. Skyline performs this task before exporting the peak area, so values greater than zero can be interpreted as positive signal for that fragment ion.

When multiple fragments of the target peptide are observed, each fragment can theoretically function as a surrogate signal for the targeted peptide ion. Each fragment can be evaluated through SRM collider, after setting the human proteome as background, under the assumption that fragments with fewer interfering fragments from the human proteome may be more specific indicators of valid CFP-10 peptide signal. We restricted ion types to b/y ions and within the 200-1,500 m/z range that represent the best resolution region of the MRM-MS instrument. This analysis indicated that there were 24 fragments of our target peptide that were suitable for MRM detection. In practice, however, only some of these ions can be observed in the MS spectrum due to incomplete fragmentation.

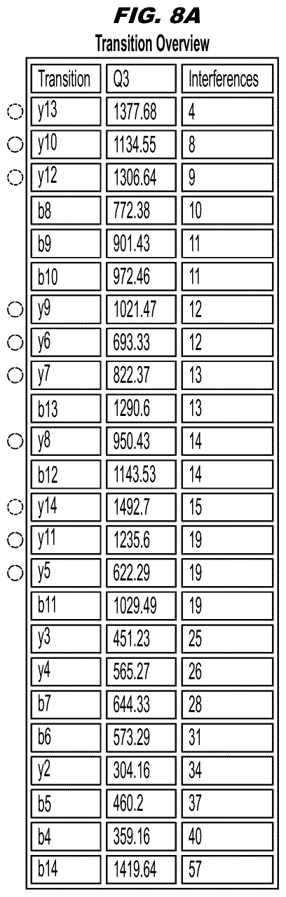

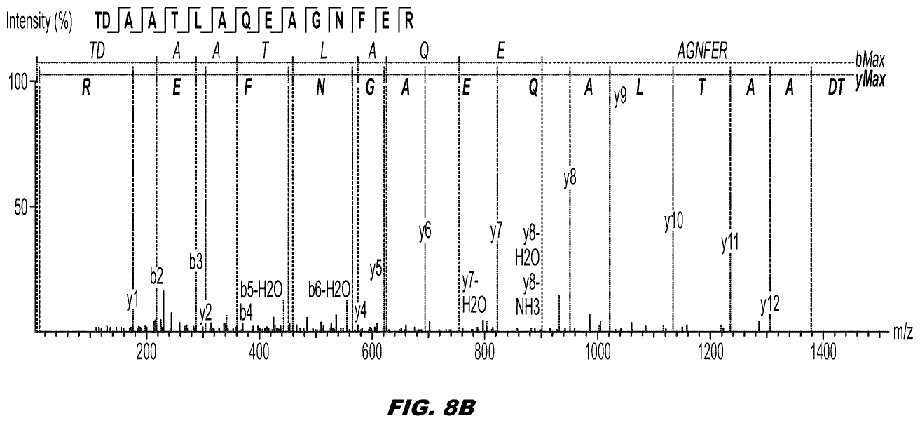

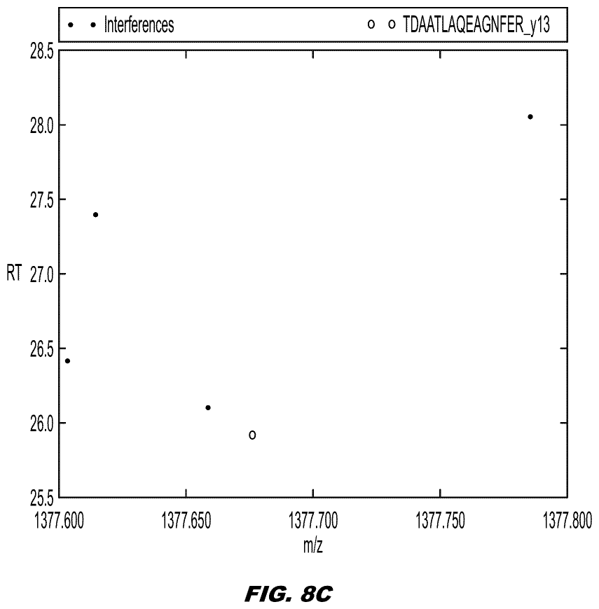

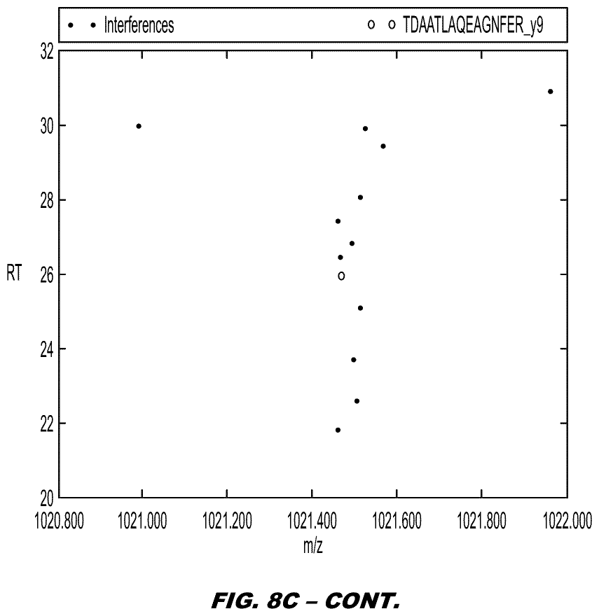

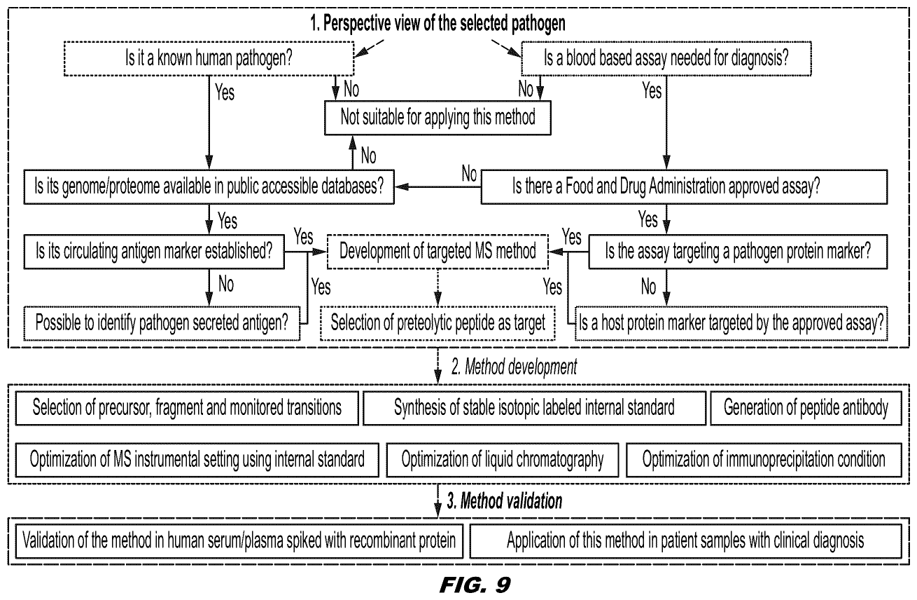

As shown in FIGS. 8A-8C, y9 is the most intense fragment ion produced by the CFP-10 peptide TDAATLAQEAGNFER (SEQ ID NO: 1), and this ion peak has 100-fold greater intensity than that of y13. Some fragments (e.g, y14 and y3) were not found on the MS/MS spectrum by the peak picking and scoring algorithm. After evaluation by SRM Collider, there are more interferences with y9 than that with y 13 (FIG. 8c). However, most of the interferences of y9are distant in their retention times, which means they are quite different in their hydrophobicity and can be easily separated by the nanoflow LC employed in this analysis. Further, only five of the examples matching the interference peptide sequence (Table 3—Exhibit B) did not have a carbamidomethylated cysteine, which was present in the remaining seven peptides.

This example indicates an advantage of our modified workflow, since its omission of the standard reduction/akylation step, removes 60% of the interference for y9 (7of 12 instances). The y9 ion is the most intense fragment produced by our target CFP-10 peptide and is highly specific for it, and therefore represent an optimal diagnostic fragment. The detection specificity for the CFP-10 target peptide can be further increased by the selection of additional product ions using the same approach.

We have developed an algorithm of how to evaluate MRM transitions and interpret their MS signal as shown in Box 1. It is based on the intensities and uniqueness of observed MRM transitions as well as their rdotp values. This approach indicates a clear rationale for evaluating whether or not an MS signal for a target peptide (e.g. our CFP-10 target peptide) can be called a true positive based on its score from this approach.

### Algorithm Optimization Based on ROCC

A training set and a validation set of MS signals from two clinical cohorts are used to further improve the algorithm for data interpretation (Box 1).

Table 2 shows MRM transition list for Mtb antigens.

| | | Start | End | | | | Collision |
|---|---|---|---|---|---|---|---|
| | SEQ | Time | Time | | Precursor | Product | Energy |
| Compound | ID NO: | (min) | (min) | Polarity | (m/z) | (m/z) | (V) |
| LYASAEATDSK_Mpt32 | 24 | 9.4 | 11.4 | Positive | 578.28 | 650.3 | 30 |
| LYASAEATDSK_Mpt32 | 24 | 9.4 | 11.4 | Positive | 578.28 | 721.34 | 30 |
| LYASAEATDSK_Mpt32 | 24 | 9.4 | 11.4 | Positive | 578.28 | 808.37 | 30 |
| LYASAEATDSK_Mpt32 | 24 | 9.4 | 11.4 | Positive | 578.28 | 879.41 | 30 |
| LYASAEATDSK_Mpt32 | 24 | 9.4 | 11.4 | Positive | 578.28 | 1042.47 | 30 |
| NDPTQQIPK_Ag85b | 25 | 13.5 | 15.5 | Positive | 520.77 | 230.08 | 30 |
| NDPTQQIPK_Ag85b | 25 | 13.5 | 15.5 | Positive | 520.77 | 244.17 | 30 |
| NDPTQQIPK_Ag85b | 25 | 13.5 | 15.5 | Positive | 520.77 | 406.24 | 30 |
| NDPTQQIPK_Ag85b | 25 | 13.5 | 15.5 | Positive | 520.77 | 613.37 | 30 |
| NDPTQQIPK_Ag85b | 25 | 13.5 | 15.5 | Positive | 520.77 | 714.41 | 30 |
| NDPTQQIPK_Ag85b | 25 | 13.5 | 15.5 | Positive | 520.77 | 811.47 | 30 |
| EsxN_AQAASLEAEHQAIVR | 26 | 15.3 | 17.3 | Positive | 797.42 | 697.87 | 30 |
| EsxN_AQAASLEAEHQAIVR | 26 | 15.3 | 17.3 | Positive | 797.42 | 723.43 | 30 |
| EsxN_AQAASLEAEHQAIVR | 26 | 15.3 | 17.3 | Positive | 797.42 | 923.51 | 30 |
| EsxN_AQAASLEAEHQAIVR | 26 | 15.3 | 17.3 | Positive | 797.42 | 1052.55 | 30 |
| EsxN_AQAASLEAEHQAIVR | 26 | 15.3 | 17.3 | Positive | 797.42 | 1165.63 | 30 |
| FLSAATSSTPR_Mpt64 | 27 | 15.4 | 17.4 | Positive | 569.3 | 547.28 | 30 |
| FLSAATSSTPR_Mpt64 | 27 | 15.4 | 17.4 | Positive | 569.3 | 648.33 | 30 |
| FLSAATSSTPR_Mpt64 | 27 | 15.4 | 17.4 | Positive | 569.3 | 719.37 | 30 |
| FLSAATSSTPR_Mpt64 | 27 | 15.4 | 17.4 | Positive | 569.3 | 790.41 | 30 |
| FLSAATSSTPR_Mpt64 | 27 | 15.4 | 17.4 | Positive | 569.3 | 877.44 | 30 |
| VEYVDVR_GlnA | 28 | 15.8 | 17.8 | Positive | 440.23 | 274.19 | 30 |
| VEYVDVR_GlnA | 28 | 15.8 | 17.8 | Positive | 440.23 | 389.21 | 30 |
| VEYVDVR_GlnA | 28 | 15.8 | 17.8 | Positive | 440.23 | 488.28 | 30 |
| VEYVDVR_GlnA | 28 | 15.8 | 17.8 | Positive | 440.23 | 651.35 | 30 |
| VEYVDVR_GlnA | 28 | 15.8 | 17.8 | Positive | 440.23 | 780.39 | 30 |
| IESENPDAVANVQAR_AcpM | 29 | 16.4 | 18.4 | Positive | 806.9 | 658.36 | 30 |
| IESENPDAVANVQAR_AcpM | 29 | 16.4 | 18.4 | Positive | 806.9 | 757.43 | 30 |
| IESENPDAVANVQAR_AcpM | 29 | 16.4 | 18.4 | Positive | 806.9 | 828.47 | 30 |
| IESENPDAVANVQAR_AcpM | 29 | 16.4 | 18.4 | Positive | 806.9 | 943.5 | 30 |
| IESENPDAVANVQAR_AcpM | 29 | 16.4 | 18.4 | Positive | 806.9 | 1040.55 | 30 |
| IESENPDAVANVQAR_AcpM | 29 | 16.4 | 18.4 | Positive | 806.9 | 1154.6 | 30 |
| GVTEETTTGVLR_SahH | 30 | 17.05 | 19.05 | Positive | 631.83 | 545.27 | 30 |
| GVTEETTTGVLR_SahH | 30 | 17.05 | 19.05 | Positive | 631.83 | 747.44 | 30 |
| GVTEETTTGVLR_SahH | 30 | 17.05 | 19.05 | Positive | 631.83 | 876.48 | 30 |
| GVTEETTTGVLR_SahH | 30 | 17.05 | 19.05 | Positive | 631.83 | 1005.52 | 30 |
| GVTEETTTGVLR_SahH | 30 | 17.05 | 19.05 | Positive | 631.83 | 1106.57 | 30 |
| LEEENPEAAQALR_AcpM | 31 | 17.2 | 19.2 | Positive | 735.365 | 629.37 | 30 |
| LEEENPEAAQALR_AcpM | 31 | 17.2 | 19.2 | Positive | 735.365 | 758.415 | 30 |
| LEEENPEAAQALR_AcpM | 31 | 17.2 | 19.2 | Positive | 735.365 | 855.47 | 30 |
| LEEENPEAAQALR_AcpM | 31 | 17.2 | 19.2 | Positive | 735.365 | 969.51 | 30 |
| LEEENPEAAQALR_AcpM | 31 | 17.2 | 19.2 | Positive | 735.365 | 1098.55 | 30 |
| LEEENPEAAQALR_AcpM | 31 | 17.2 | 19.2 | Positive | 735.365 | 1227.6 | 30 |
| NOAETLVYQTEK_DnaK | 32 | 17.3 | 19.3 | Positive | 712.357 | 668.33 | 30 |
| NQAETLVYQTEK_DnaK | 32 | 17.3 | 19.3 | Positive | 712.357 | 767.39 | 30 |
| NQAETLVYQTEK_DnaK | 32 | 17.3 | 19.3 | Positive | 712.357 | 981.53 | 30 |
| NQAETLVYQTEK_DnaK | 32 | 17.3 | 19.3 | Positive | 712.357 | 1110.57 | 30 |
| NQAETLVYQTEK_DnaK | 32 | 17.3 | 19.3 | Positive | 712.357 | 1181.6 | 30 |
| GSLVEGGIGGTEAR_cfp2 | 33 | 17.6 | 19.6 | Positive | 651.84 | 590.29 | 30 |
| GSLVEGGIGGTEAR_cfp2 | 33 | 17.6 | 19.6 | Positive | 651.84 | 703.37 | 30 |
| GSLVEGGIGGTEAR_cfp2 | 33 | 17.6 | 19.6 | Positive | 651.84 | 760.39 | 30 |
| GSLVEGGIGGTEAR_cfp2 | 33 | 17.6 | 19.6 | Positive | 651.84 | 817.42 | 30 |
| GSLVEGGIGGTEAR_cfp2 | 33 | 17.6 | 19.6 | Positive | 651.84 | 946.46 | 30 |
| EsxO_AQAGLLEAEHQAIVR | 34 | 19.8 | 21.8 | Positive | 803.44 | 703.9 | 30 |
| EsxO_AQAGLLEAEHQAIVR | 34 | 19.8 | 21.8 | Positive | 803.44 | 723.43 | 30 |
| EsxO_AQAGLLEAEHQAIVR | 34 | 19.8 | 21.8 | Positive | 803.44 | 852.47 | 30 |
| EsxO_AQAGLLEAEHQAIVR | 34 | 19.8 | 21.8 | Positive | 803.44 | 923.51 | 30 |
| EsxO_AQAGLLEAEHQAIVR | 34 | 19.8 | 21.8 | Positive | 803.44 | 1052.55 | 30 |
| EsxO_AQAGLLEAEHQAIVR | 34 | 19.8 | 21.8 | Positive | 803.44 | 1165.63 | 30 |
| SLADPNVSFANK_cfp2 | 35 | 19.9 | 21.9 | Positive | 631.82 | 479.26 | 30 |
| SLADPNVSFANK_cfp2 | 35 | 19.9 | 21.9 | Positive | 631.82 | 566.29 | 30 |
| SLADPNVSFANK_cfp2 | 35 | 19.9 | 21.9 | Positive | 631.82 | 665.36 | 30 |
| SLADPNVSFANK_cfp2 | 35 | 19.9 | 21.9 | Positive | 631.82 | 876.46 | 30 |
| SLADPNVSFANK_cfp2 | 35 | 19.9 | 21.9 | Positive | 631.82 | 1062.52 | 30 |
| IALFGNHAPK_PpiA | 36 | 20 | 22 | Positive | 534.3 | 452.26 | 30 |
| IALFGNHAPK_PpiA | 36 | 20 | 22 | Positive | 534.3 | 566.3 | 30 |
| IALFGNHAPK_PpiA | 36 | 20 | 22 | Positive | 534.3 | 623.33 | 30 |
| IALFGNHAPK_PpiA | 36 | 20 | 22 | Positive | 534.3 | 770.39 | 30 |
| IALFGNHAPK_PpiA | 36 | 20 | 22 | Positive | 534.3 | 883.48 | 30 |
| cfp10_QELDEISTNIR | 37 | 20.65 | 22.65 | Positive | 659.34 | 503.29 | 30 |
| cfp10_QELDEISTNIR | 37 | 20.65 | 22.65 | Positive | 659.34 | 590.33 | 30 |
| cfp10_QELDEISTNIR | 37 | 20.65 | 22.65 | Positive | 659.34 | 703.41 | 30 |
| cfp10_QELDEISTNIR | 37 | 20.65 | 22.65 | Positive | 659.34 | 832.45 | 30 |
| cfp10_QELDEISTNIR | 37 | 20.65 | 22.65 | Positive | 659.34 | 947.48 | 30 |
| cfp10_QELDEISTNIR | 37 | 20.65 | 22.65 | Positive | 659.34 | 1060.56 | 30 |
| SLENYIAQTR_Mpt64 | 38 | 20.7 | 22.7 | Positive | 597.81 | 475.26 | 30 |
| SLENYIAQTR_Mpt64 | 38 | 20.7 | 22.7 | Positive | 597.81 | 588.35 | 30 |
| SLENYIAQTR_Mpt64 | 38 | 20.7 | 22.7 | Positive | 597.81 | 751.41 | 30 |
| SLENYIAQTR_Mpt64 | 38 | 20.7 | 22.7 | Positive | 597.81 | 865.45 | 30 |
| SLENYIAQTR_Mpt64 | 38 | 20.7 | 22.7 | Positive | 597.81 | 994.5 | 30 |
| TTGDPPFPGQPPPVANDTR_Mpt32 | 39 | 21.4 | 23.4 | Positive | 655.32 | 483.75 | 30 |
| TTGDPPFPGQPPPVANDTR_Mpt32 | 39 | 21.4 | 23.4 | Positive | 655.32 | 772.39 | 30 |
| TTGDPPFPGQPPPVANDTR_Mpt32 | 39 | 21.4 | 23.4 | Positive | 655.32 | 795.41 | 30 |
| TTGDPPFPGQPPPVANDTR_Mpt32 | 39 | 21.4 | 23.4 | Positive | 655.32 | 869.45 | 30 |
| TTGDPPFPGQPPPVANDTR_Mpt32 | 39 | 21.4 | 23.4 | Positive | 655.32 | 966.5 | 30 |
| TTGDPPFPGQPPPVANDTR_Mpt32 | 39 | 21.4 | 23.4 | Positive | 655.32 | 998.46 | 30 |
| IPDEDLAGLR_AcpM | 40 | 21.6 | 23.6 | Positive | 549.79 | 493.25 | 30 |
| IPDEDLAGLR_AcpM | 40 | 21.6 | 23.6 | Positive | 549.79 | 529.35 | 30 |
| IPDEDLAGLR_AcpM | 40 | 21.6 | 23.6 | Positive | 549.79 | 644.37 | 30 |
| IPDEDLAGLR_AcpM | 40 | 21.6 | 23.6 | Positive | 549.79 | 773.415 | 30 |
| IPDEDLAGLR_AcpM | 40 | 21.6 | 23.6 | Positive | 549.79 | 888.44 | 30 |
| IPDEDLAGLR_AcpM | 40 | 21.6 | 23.6 | Positive | 549.79 | 985.49 | 30 |
| TVANFVGLAQGTK_PpiA | 41 | 23.2 | 25.2 | Positive | 653.36 | 504.28 | 30 |
| TVANFVGLAQGTK_PpiA | 41 | 23.2 | 25.2 | Positive | 653.36 | 674.38 | 30 |
| TVANFVGLAQGTK_PpiA | 41 | 23.2 | 25.2 | Positive | 653.36 | 773.44 | 30 |
| TVANFVGLAQGTK_PpiA | 41 | 23.2 | 25.2 | Positive | 653.36 | 1034.56 | 30 |
| TVANFVGLAQGTK_PpiA | 41 | 23.2 | 25.2 | Positive | 653.36 | 1105.6 | 30 |
| LVFLTGPK_GarA | 42 | 23.5 | 25.5 | Positive | 437.77 | 301.19 | 30 |
| LVFLTGPK_GarA | 42 | 23.5 | 25.5 | Positive | 437.77 | 402.22 | 30 |
| LVFLTGPK_GarA | 42 | 23.5 | 25.5 | Positive | 437.77 | 515.32 | 30 |
| LVFLTGPK_GarA | 42 | 23.5 | 25.5 | Positive | 437.77 | 662.39 | 30 |
| LVFLTGPK_GarA | 42 | 23.5 | 25.5 | Positive | 437.77 | 761.46 | 30 |
| MPAVTDLVK_DnaK | 43 | 23.5 | 25.5 | Positive | 487.27 | 421.75 | 30 |
| MPAVTDLVK_DnaK | 43 | 23.5 | 25.5 | Positive | 487.27 | 575.34 | 30 |
| MPAVTDLVK_DnaK | 43 | 23.5 | 25.5 | Positive | 487.27 | 674.41 | 30 |
| MPAVTDLVK_DnaK | 43 | 23.5 | 25.5 | Positive | 487.27 | 745.45 | 30 |
| MPAVTDLVK_DnaK | 43 | 23.5 | 25.5 | Positive | 487.27 | 842.5 | 30 |
| AFDWDQAYR_Mpt64 | 44 | 24 | 26 | Positive | 586.26 | 409.22 | 30 |
| AFDWDQAYR_Mpt64 | 44 | 24 | 26 | Positive | 586.26 | 537.28 | 30 |
| AFDWDQAYR_Mpt64 | 44 | 24 | 26 | Positive | 586.26 | 652.3 | 30 |
| AFDWDQAYR_Mpt64 | 44 | 24 | 26 | Positive | 586.26 | 838.38 | 30 |
| AFDWDQAYR_Mpt64 | 44 | 24 | 26 | Positive | 586.26 | 953.41 | 30 |
| TVGDVVAYIQK_AcpM | 45 | 24 | 26 | Positive | 596.832 | 551.32 | 30 |
| TVGDVVAYIQK_AcpM | 45 | 24 | 26 | Positive | 596.832 | 622.36 | 30 |
| TVGDVVAYIQK_AcpM | 45 | 24 | 26 | Positive | 596.832 | 721.42 | 30 |
| TVGDVVAYIQK_AcpM | 45 | 24 | 26 | Positive | 596.832 | 820.49 | 30 |
| TVGDVVAYIQK AcpM | 45 | 24 | 26 | Positive | 596.832 | 935.52 | 30 |
| TVGDVVAYIQK_AcpM | 45 | 24 | 26 | Positive | 596.832 | 992.54 | 30 |
| cfp10_TDAATLAQEAGNFER | 46 | 24.3 | 26.3 | Positive | 797.379 | 693.33 | 30 |
| cfp10_TDAATLAQEAGNFER | 46 | 24.3 | 26.3 | Positive | 797.379 | 822.37 | 30 |
| cfp10_TDAATLAQEAGNFER | 46 | 24.3 | 26.3 | Positive | 797.379 | 950.43 | 30 |
| cfp10_TDAATLAQEAGNFER | 46 | 24.3 | 26.3 | Positive | 797.379 | 1021.47 | 30 |
| cfp10_TDAATLAQEAGNFER | 46 | 24.3 | 26.3 | Positive | 797.379 | 1134.55 | 30 |
| cfp10_TDAATLAQEAGNFER | 46 | 24.3 | 26.3 | Positive | 797.379 | 1235.6 | 30 |
| VVDWLVDK_DnaK | 47 | 24.5 | 26.5 | Positive | 487.27 | 361.21 | 30 |
| VVDWLVDK_DnaK | 47 | 24.5 | 26.5 | Positive | 487.27 | 474.29 | 30 |
| VVDWLVDK_DnaK | 47 | 24.5 | 26.5 | Positive | 487.27 | 660.37 | 30 |
| VVDWLVDK_DnaK | 47 | 24.5 | 26.5 | Positive | 487.27 | 775.4 | 30 |
| VVDWLVDK_DnaK | 47 | 24.5 | 26.5 | Positive | 487.27 | 874.47 | 30 |
| cfp10_TQIDQVESTAGSLQGQWR | 48 | 25.3 | 27.3 | Positive | 1002.493 | 787.42 | 30 |
| cfp10_TQIDQVESTAGSLQGQWR | 48 | 25.3 | 27.3 | Positive | 1002.493 | 931.47 | 30 |
| cfp10_TQIDQVESTAGSLQGQWR | 48 | 25.3 | 27.3 | Positive | 1002.493 | 1103.56 | 30 |
| cfp10_TQIDQVESTAGSLQGQWR | 48 | 25.3 | 27.3 | Positive | 1002.493 | 1190.61 | 30 |
| cfp10_TQIDQVESTAGSLQGQWR | 48 | 25.3 | 27.3 | Positive | 1002.493 | 1319.63 | 30 |
| AADMWGPSSDPAWER_Ag85B | 49 | 25.4 | 27.4 | Positive | 838.36 | 389.15 | 30 |
| AADMWGPSSDPAWER_Ag85B | 49 | 25.4 | 27.4 | Positive | 838.36 | 575.23 | 30 |
| AADMWGPSSDPAWER_Ag85B | 49 | 25.4 | 27.4 | Positive | 838.36 | 658.33 | 30 |
| AADMWGPSSDPAWER_Ag85B | 49 | 25.4 | 27.4 | Positive | 838.36 | 1044.47 | 30 |
| AADMWGPSSDPAWER_Ag85B | 49 | 25.4 | 27.4 | Positive | 838.36 | 1101.5 | 30 |
| AADMWGPSSDPAWER_Ag85B | 49 | 25.4 | 27.4 | Positive | 838.36 | 1287.58 | 30 |
| IPLDVAEGDTVIYSK_GroS | 50 | 26.2 | 28.2 | Positive | 810.43 | 882.46 | 30 |
| IPLDVAEGDTVIYSK_GroS | 50 | 26.2 | 28.2 | Positive | 810.43 | 1011.5 | 30 |
| IPLDVAEGDTVIYSK_GroS | 50 | 26.2 | 28.2 | Positive | 810.43 | 1082.54 | 30 |
| IPLDVAEGDTVIYSK_GroS | 50 | 26.2 | 28.2 | Positive | 810.43 | 1181.6 | 30 |
| IPLDVAEGDTVIYSK_GroS | 50 | 26.2 | 28.2 | Positive | 810.43 | 1296.63 | 30 |
| FSDPSKPNGQIWTGVIGSPA | 51 | 27.2 | 29.2 | Positive | 1044.85 | 625.34 | 30 |
| ANAPDAGPPQR_Mpt32 | | | | | | | |
| FSDPSKPNGQIWTGVIGSPA | 51 | 27.2 | 29.2 | Positive | 1044.85 | 837.42 | 30 |
| ANAPDAGPPQR_Mpt32 | | | | | | | |
| FSDPSKPNGQIWTGVIGSPA | 51 | 27.2 | 29.2 | Positive | 1044.85 | 1022.5 | 30 |
| ANAPDAGPPQR_Mpt32 | | | | | | | |
| FSDPSKPNGQIWTGVIGSPA | 51 | 27.2 | 29.2 | Positive | 1044.85 | 1164.58 | 30 |
| ANAPDAGPPQR_Mpt32 | | | | | | | |
| FSDPSKPNGQIWTGVIGSPA | 51 | 27.2 | 29.2 | Positive | 1044.85 | 1261.63 | 30 |
| ANAPDAGPPQR Mpt32 | | | | | | | |
| FLLDQAITSAGR_GarA | 52 | 27.7 | 29.7 | Positive | 646.35 | 604.34 | 30 |
| FLLDQAITSAGR_GarA | 52 | 27.7 | 29.7 | Positive | 646.35 | 675.38 | 30 |
| FLLDQAITSAGR_GarA | 52 | 27.7 | 29.7 | Positive | 646.35 | 803.44 | 30 |
| FLLDQAITSAGR_GarA | 52 | 27.7 | 29.7 | Positive | 646.35 | 918.47 | 30 |
| FLLDQAITSAGR_GarA | 52 | 27.7 | 29.7 | Positive | 646.35 | 1031.55 | 30 |
| EAPYELNITSATYQSAIPPR_Mpt64 | 53 | 28.4 | 30.4 | Positive | 1111.06 | 931.5 | 30 |
| EAPYELNITSATYQSAIPPR_Mpt64 | 53 | 28.4 | 30.4 | Positive | 1111.06 | 1032.55 | 30 |
| EAPYELNITSATYQSAIPPR_Mpt64 | 53 | 28.4 | 30.4 | Positive | 1111.06 | 1103.58 | 30 |
| EAPYELNITSATYQSAIPPR_Mpt64 | 53 | 28.4 | 30.4 | Positive | 1111.06 | 1190.62 | 30 |
| EAPYELNITSATYQSAIPPR_Mpt64 | 53 | 28.4 | 30.4 | Positive | 1111.06 | 1291.66 | 30 |
| EAPYELNITSATYQSAIPPR_Mpt64 | 53 | 28.4 | 30.4 | Positive | 1111.06 | 1404.75 | 30 |
| GFQSIHESDMLLLPDPETAR_GlnA | 54 | 29 | 31 | Positive | 752.7 | 573.3 | 30 |
| GFQSIHESDMLLLPDPETAR_GlnA | 54 | 29 | 31 | Positive | 752.7 | 785.38 | 30 |
| GFQSIHESDMLLLPDPETAR_GlnA | 54 | 29 | 31 | Positive | 752.7 | 898.46 | 30 |
| GFQSIHESDMLLLPDPETAR_GlnA | 54 | 29 | 31 | Positive | 752.7 | 1011.43 | 30 |
| GFQSIHESDMLLLPDPETAR_GlnA | 54 | 29 | 31 | Positive | 752.7 | 1132.47 | 30 |
| GFQSIHESDMLLLPDPETAR_GlnA | 54 | 29 | 3 | Positive | 752.7 | 1245.56 | 30 |
| SVFDDGLAFDGSSIR_GlnA | 55 | 30.1 | 32.1 | Positive | 793.378 | 781.38 | 30 |
| SVFDDGLAFDGSSIR_GlnA | 55 | 30.1 | 32.1 | Positive | 793.378 | 852.42 | 30 |
| SVFDDGLAFDGSSIR_GlnA | 55 | 30.1 | 32.1 | Positive | 793.378 | 965.51 | 30 |
| SVFDDGLAFDGSSIR_GlnA | 55 | 30.1 | 32.1 | Positive | 793.378 | 1022.53 | 30 |
| SVFDDGLAFDGSSIR_GlnA | 55 | 30.1 | 32.1 | Positive | 793.378 | 1137.55 | 30 |
| SVFDDGLAFDGSSIR_GlnA | 55 | 30.1 | 32.1 | Positive | 793.378 | 1252.58 | 30 |
| PGLPVEYLQVPSPSMGR_Ag85B | 56 | 31.1 | 33.1 | Positive | 913.97 | 731.87 | 30 |
| PGLPVEYLQVPSPSMGR_Ag85B | 56 | 31.1 | 33.1 | Positive | 913.97 | 830.42 | 30 |
| PGLPVEYLQVPSPSMGR_Ag85B | 56 | 31.1 | 33.1 | Positive | 913.97 | 958.48 | 30 |
| PGLPVEYLQVPSPSMGR_Ag85B | 56 | 31.1 | 33.1 | Positive | 913.97 | 1071.56 | 30 |
| PGLPVEYLQVPSPSMGR_Ag85B | 56 | 31.1 | 33.1 | Positive | 913.97 | 1234.62 | 30 |
| PGLPVEYLQVPSPSMGR_Ag85B | 56 | 31.1 | 33.1 | Positive | 913.97 | 1363.67 | 30 |
| ESAT6_WDATATELNNALQNLAR | 57 | 33.6 | 35.6 | Positive | 950.98 | 601.34 | 30 |
| ESAT6_WDATATELNNALQNLAR | 57 | 33.6 | 35.6 | Positive | 950.98 | 899.51 | 30 |
| ESAT6_WDATATELNNALQNLAR | 57 | 33.6 | 35.6 | Positive | 950.98 | 1013.55 | 30 |
| ESAT6_WDATATELNNALQNLAR | 57 | 33.6 | 35.6 | Positive | 950.98 | 1126.63 | 30 |
| ESAT6_WDATATELNNALQNLAR | 57 | 33.6 | 35.6 | Positive | 950.98 | 1255.68 | 30 |
| AGANLFELENFVAR_Bfrb | 58 | 33.7 | 35.7 | Positive | 775.9 | 735.39 | 30 |
| AGANLFELENFVAR_Bfrb | 58 | 33.7 | 35.7 | Positive | 775.9 | 848.46 | 30 |
| AGANLFELENFVAR_Bfrb | 58 | 33.7 | 35.7 | Positive | 775.9 | 977.51 | 30 |
| AGANLFELENFVAR_Bfrb | 58 | 33.7 | 35.7 | Positive | 775.9 | 1124.57 | 30 |
| AGANLFELENFVAR_Bfrb | 58 | 33.7 | 35.7 | Positive | 775.9 | 1237.66 | 30 |
| WETFLTSELPGWLQANR_Ag85A | 59 | 38.1 | 40.1 | Positive | 1024.52 | 417.1 | 30 |
| WETFLTSELPGWLQANR_Ag85A | 59 | 38.1 | 40.1 | Positive | 1024.52 | 941.5 | 30 |
| WETFLTSELPGWLQANR_Ag85A | 59 | 38.1 | 40.1 | Positive | 1024.52 | 1054.58 | 30 |
| WETFLTSELPGWLQANR_Ag85A | 59 | 38.1 | 40.1 | Positive | 1024.52 | 1183.62 | 30 |
| WETFLTSELPGWLQANR_Ag85A | 59 | 38.1 | 40.1 | Positive | 1024.52 | 1270.65 | 30 |
| Cfp2_DPASAPDVPTAAQLTSLLNSL | 60 | 38.9 | 40.9 | Positive | 1042.2 | 566.29 | 30 |
| ADPNVSFANK | | | | | | | |
| Cfp2_DPASAPDVPTAAQLTSLLNSL | 60 | 38.9 | 40.9 | Positive | 1042.2 | 753.34 | 30 |
| ADPNVSFANK | | | | | | | |
| Cfp2_DPASAPDVPTAAQLTSLLNSL | 60 | 38.9 | 40.9 | Positive | 1042.2 | 876.46 | 30 |
| ADPNVSFANK | | | | | | | |
| Cfp2_DPASAPDVPTAAQLTSLLNSL | 60 | 38.9 | 40.9 | Positive | 1042.2 | 991.48 | 30 |
| ADPNVSFANK | | | | | | | |
| Cfp2_DPASAPDVPTAAQLTSLLNSL | 60 | 38.9 | 40.9 | Positive | 1042.2 | 1062.52 | 30 |
| ADPNVSFANK | | | | | | | |

TABLE 3
[]interference ions for each transition.

| Q1 | RT | Sequence | SEQ ID NO: | Transitions |
|---|---|---|---|---|
| 797.42 | 27.45 | TNLTTEQIANYVAR | 61 | y14 y6 y5 y3 |
| | | | | b9 b10 b14 |
| 796.41 | 26.68 | DPALC[160]QHKPLTPQGDELSKP | 62 | y8 y7 y3 b8 |
| | | R | | b9 b11 |
| 797.42 | 26.7 | LGGLSAPPWAKPEDR | 63 | y8 y6 y3 b7 |
| | | | | b9 b14 |
| 796.89 | 22.32 | DLEVETLTASSEGNK | 64 | y11 y3 b6 b9 |
| | | | | b10 |
| 797.44 | 27.7 | TTLPGVVNGANNPAIR | 65 | y14 y12 y11 |
| | | | | b4 b14 |
| 797.4 | 28.15 | YSDIEPSTGGEVVLK | 66 | y11 y8 y2 b4 |
| | | | | b7 |
| 797.38 | 29.01 | QYHVQFFGDAPER | 67 | y8 y2 b7 b13 |
| | | | | b14 |
| 796.4 | 29.5 | MTSEIETNIVAVER | 68 | y3 y2 b6 b9 |
| | | | | b12 |
| 797.39 | 21.07 | MEPGPDGPAASGPAAIR | 69 | y12 y11 b4 |
| | | | | b14 |
| 796.04 | 21.36 | VNDPTESQQEDQLIAGAQDEAK | 70 | y7 y5 b11 |
| | | | | b12 |
| 797.41 | 22.49 | EQVQIGAHSPPQFR | 71 | y8 b6 b7 b14 |
| 797.38 | 24.24 | TEQEVVEGMDISTR | 72 | y14 y11 b4 |
| | | | | b14 |
| 796.39 | 25.46 | VVC[160]TESWPLAHHR | 73 | y14 b4 b5 |
| | | | | b12 |
| 797.43 | 26.45 | EPGPIAPSTNSSPVLK | 74 | y11 y4 b4 |
| | | | | b11 |
| 795.88 | 26.57 | AHFNAMFQPSSPTR | 75 | y11 b5 b7 b8 |
| 797.37 | 26.75 | MDQFGNGLEIDQAR | 76 | y6 y3 b9 b14 |
| 796.73 | 28.69 | DPALC[160]QHKPLTPQGDELSEP | 77 | y8 y7 y3 b8 |
| | | R | | |
| 797.07 | 29.06 | METRPTALMSSTVAAAAPAAGA | 78 | y12 y3 b4 |
| | | ASR | | b11 |
| 796.43 | 29.4 | TRPTDLVFVVDSSR | 79 | y9 y5 y4 b11 |
| 796.89 | 29.92 | ELGFVQPSGVTDGMR | 80 | y7 b5 b6 b7 |
| 795.88 | 30.82 | MLTDPDLPQEFER | 81 | y3 y2 b5 b7 |
| 795.89 | 20.98 | C[160]STPLLHQQYTSR | 82 | y5 b5 b6 |
| 796.4 | 21.15 | ISC[160]GPPAHVENAIAR | 83 | b4 b5 b8 |
| 797.41 | 22.12 | LLEAVGSSSGTPNAPPP | 84 | y6 y3 b9 |
| 797.71 | 22.51 | TPTQHSPVPPEEVTGPSQMDTR | 85 | y7 y4 b8 |
| 797.42 | 22.79 | TTYVSQSGQVISAPR | 86 | y14 y4 b14 |
| 797.4 | 22.98 | SFPQSSQLSQETVR | 87 | y10 b5 b14 |
| 795.87 | 23.43 | MNGDHMVLGSSVTDK | 88 | y6 y4 b7 |
| 797.38 | 23.66 | EYPLYINQTC[160]HR | 89 | y9 b6 b14 |
| 797.37 | 24.12 | GEGAIGSLDYTPEER | 90 | y2 b13 b14 |
| 797.39 | 24.58 | DVVHPLGGEEPSMAR | 91 | y3 b12 b14 |
| 797.38 | 25.1 | VYQPFLTTC[160]DGHR | 92 | y8 b7 b14 |
| 797.36 | 25.19 | AFDC[160]PSSFQIHER | 93 | y2 b13 b14 |
| 796.4 | 26.1 | TLAPQVC[160]SSFATGPR | 94 | y13 y12 y7 |
| 797.37 | 26.24 | SEPC[160]DDLQIPNTNVHLSHDA | 95 | y11 b9 b11 |
| | | K | | |
| 796.39 | 26.39 | FNNVQLNLTDEER | 96 | y2 b4 b7 |
| 796.39 | 26.48 | QLVDEFQASGGVGER | 97 | y2 b5 b7 |
| 797.04 | 26.88 | EDASGQLSC[160]IQLPVDSQGGD | 98 | b5 b9 b11 |
| | | ANK | | |
| 797.34 | 26.92 | FTSDMSNTEWGYR | 99 | y3 b12 b14 |
| 797.36 | 27.1 | MAFMAATDHSDQLR | 100 | y12 y5 b14 |
| 797.41 | 27.17 | LEIGPVYSSVSSEAR | 101 | y7 b8 b14 |
| 796.38 | 27.4 | TIGMPATEEVDC[160]IR | 102 | y13 y9 bil |
| 797.41 | 27.63 | LLETVEYNISGAER | 103 | y2 b13 b14 |
| 797.38 | 27.99 | SQGDNNVSLVEEFR | 104 | y3 b12 b14 |
| 796.46 | 28.05 | TIQAPTQVPVVVSPR | 105 | y13 y10 b4 |
| 797.41 | 28.06 | C(160}TLHLGIEPPDSVR | 106 | y9 b6 b14 |
| 796.41 | 28.21 | SEITNQLSVSDINSQSVGGARPK | 107 | b7 b10 b11 |
| 797.72 | 29.09 | WAGGPPGTGGHGPLSLNSPDPYE | 108 | y7 y5 y4 |
| | | K | | |
| 796.39 | 29.1 | GC[160]FTPVVTDPITER | 109 | y2 b7 b11 |
| 797.37 | 30.38 | GSIDDVFNC[160]NLSPR | 110 | y11 b4 b14 |
| 796.37 | 30.43 | TFISLSSTDVSPNQSNTSNEMK | 111 | y11 y2 b12 |
| 796.07 | 30.61 | HVLTSHIDEPPTQNQSDLLNK | 112 | y8 y4 y3 |
| 795.89 | 30.85 | STLGPALEAVSMDGDK | 113 | y7 y4 b4 |
| 797.4 | 21.2 | VWTHC[160]QTQHGIVK | 114 | y11 b4 |
| 795.88 | 21.4 | QSGQC[160]LDGVSLSSPR | 115 | b4 b6 |
| 797.35 | 21.54 | ENYGSITSMGYESR | 116 | y4 b14 |
| 797.37 | 21.58 | EGDYIVSVNGQPC[160]R | 117 | y4 b14 |
| 796.41 | 21.7 | EGYLQIGANTQAAQK | 118 | y5 y4 |
| 795.88 | 21.78 | TNGVPTTEEVDC[160]IR | 119 | y9 b12 |
| 797.44 | 22.47 | VQTDKPHLVSLGSGR | 120 | b5 b14 |
| 796.36 | 22.49 | YSSNLGNFNYEQR | 121 | y5 y4 |
| 796.72 | 22.54 | VVAGDHNLSQNDGTEQYVSVQK | 122 | y9 y6 |
| 797.39 | 22.71 | ILDETQEAVEYQR | 123 | y4 b14 |
| 797.02 | 22.83 | DDSC[160]SGDSSAQLSSGEHLLG | 124 | y5 y4 |
| | | PNR | | |
| 795.88 | 23.03 | SVQWC[160]AVSQPEATK | 125 | b5 b12 |
| 795.88 | 23.36 | NTSSEQEEVVEALR | 126 | b4 b7 |
| 796.9 | 23.42 | TTTWQRPTMESVR | 127 | y2 b10 |
| 797.4 | 23.55 | TASESISNLSEAGSIK | 128 | y14 b5 |
| 797.41 | 23.71 | TPSTTTSSHYLGTLK | 129 | y14 b5 |
| 797.42 | 24.1 | TVGFNHLTLGHNQR | 130 | y14 b14 |
| 796.34 | 24.12 | GGHINDAFMTEDER | 131 | y2 b7 |
| 797.3 | 24.31 | GFGC[160]C[160]FPC[160]C[160]SV | 132 | y2 b6 |
| | | DK | | |
| 796.41 | 24.31 | WTC[160]SKPKPSTMLR | 133 | y2 b6 |
| 796.86 | 24.36 | MPAYHSSLMDPDTK | 134 | y4 b5 |
| 797.36 | 24.44 | SSLGLDNSLSTSSEDPHSGC[160]P | 135 | b4 b6 |
| | | SR | | |
| 797.7 | 24.88 | GSEC[160]WHLSSGSVHPSPGSAP | 136 | b7 b13 |
| | | AQR | | |
| 796.91 | 25.05 | C[160]QPLGSALPPQAPTR | 137 | y9 y8 |
| 797.42 | 25.15 | NIQHLNSQIHSFR | 138 | y2 b14 |
| 795.9 | 25.18 | VTTSDEDIGINAISR | 139 | b5 b6 |
| 797.38 | 25.36 | FVELQVC[160]DHYQR | 140 | b4 b14 |
| 797.37 | 25.65 | HFITSSSSKPC[160]EPEEHYVQK | 141 | b8 b11 |
| 797.38 | 25.71 | TTGSTQSNFNFYVK | 142 | y14 b6 |
| 797.03 | 25.82 | GLHGAATVVLGQGQHGGC[160] | 143 | y2 b5 |
| | | APEEED | | |
| 796.86 | 25.85 | TLTAEEAEEEWER | 144 | y2 b4 |
| 796.93 | 26.14 | FGGTPIHFPGGRPPR | 145 | b5 b6 |
| 796.37 | 26.41 | TLDENSDSAGLWQR | 146 | y13 b6 |
| 797.04 | 26.42 | LNVAGAGGGGDSGGGPGAATDG | 147 | y6 b11 |
| | | NFTAGLA | | |
| 795.91 | 26.52 | VFSQNAYLIDHQR | 148 | y4 b7 |
| 796.92 | 26.66 | EGSGNPTPLINPLAGR | 149 | y8 b13 |
| 797.41 | 26.69 | TFAQTTYLIDHQR | 150 | y14 b14 |
| 796.89 | 26.73 | VASMAPVTAEGFQER | 151 | y2 b5 |
| 796.42 | 26.79 | SVLPPDGNGSPVLPDK | 152 | y9 b4 |
| 797.45 | 26.98 | VNPDLQVEVKPSIR | 153 | y12 b14 |
| 797.71 | 27.08 | TSETNTPQGNQEQLVTVMEER | 154 | y2 b7 |
| 797.41 | 27.36 | QIQGTETEFNSLVK | 155 | y11 b4 |
| 796.89 | 27.47 | DEIYIPLQEEDTK | 156 | y11 b10 |
| 795.87 | 27.53 | VEDESLDNTWLNR | 157 | y3 b5 |
| 797.37 | 27.67 | C[160]VPRPGGAVC[160]EC[160]P | 158 | y8 b11 |
| | | GGFQLDASR | | |
| 796.85 | 27.67 | MGGGGALQWNC[160]SGGIQ | 159 | b10 b13 |
| 796.41 | 27.77 | DVEEKPAHAPARPEAPVDSMLK | 160 | y5 b6 |
| 795.9 | 27.91 | MAGSYPEGAPAILADK | 161 | y2 b6 |
| 796.37 | 28.35 | LDC[160]ASAIQNYLSGTHGGSPG | 162 | y2 b5 |
| | | PER | | |
| 796.91 | 28.68 | HALPSPLEGSFQPGR | 163 | y6 b4 |
| 797.38 | 28.69 | HC[160]PSTFFSSPGLTR | 164 | b4 b14 |
| 796.87 | 28.81 | ISDDTPLEMMTSPR | 165 | b4 b5 |
| 796.32 | 28.9 | ELC[160]DC[160]EQC[160]GEVFSE | 166 | y11 b7 |
| | | HSC[160]LK | | |
| 797.41 | 29.06 | EGVYEISLSPTGVSR | 167 | b6 b14 |
| 795.89 | 29.13 | SESEVHFDVETAIK | 168 | y2 b7 |
| 796.43 | 29.18 | LIGTNC[160]IIYPVNSK | 169 | b6 b8 |
| 795.9 | 29.42 | C[160]DVVVVGGGISGMAAAK | 170 | b5 b6 |
| 796.91 | 29.6 | EEVYIVQASNVDVK | 171 | y11 b5 |
| 797.4 | 29.62 | QEAASTGPGMEPETTATTILASV | 172 | y6 b11 |
| | | K | | |
| 796.4 | 29.71 | VVETMQSTLDAEIR | 173 | y14 b4 |
| 797.45 | 29.92 | INSELQVPPTQVLR | 174 | y12 b14 |
| 796.4 | 29.96 | QQNAQGGFSSTQDTVVALHALSK | 175 | y9 bS |
| 796.41 | 30.08 | FPSLLTQNENMVAK | 176 | y3 b9 |
| 796.07 | 30.14 | QHHHQSSFPGSLPQETNLTLK | 177 | y5 b12 |
| 796.72 | 30.18 | SGGEALAVANDSTSTPQNANGLW | 178 | y4 b11 |
| | | K | | |
| 797.34 | 30.31 | SSC[160]SDMDLLHSWR | 179 | b6 b14 |
| 796.38 | 20.94 | AGSVHYGHYTALC[160]R | 180 | b10 |
| 796.37 | 21.03 | C[160]NTQAELLAAGC[160]QR | 181 | y4 |
| 795.85 | 21.1 | GVLVC[160]DEC[160]C[160]SVHR | 182 | b7 |
| 795.8 | 21.1 | TFMDMDQDSEDEK | 183 | y2 |
| 796.85 | 21.18 | LTWHSYPSEDDDK | 184 | b10 |
| 796.91 | 21.19 | FAVVENNSSAVTAQR | 185 | b13 |
| 796.85 | 21.19 | VEDTC[160]VEWDPTGGK | 186 | y10 |
| 795.89 | 21.32 | AFSQNSQFIQHQR | 187 | b7 |
| 795.9 | 21.34 | C[160]LPNPTPEGGAVPGPK | 188 | b13 |
| 796.84 | 21.35 | GEGQGLVC[160]DLC[160]NDR | 189 | b11 |
| 796.86 | 21.44 | SSTNTSLPDDNGAWK | 190 | y3 |
| 795.91 | 21.46 | LGEVVNTHGPVEPDK | 191 | b4 |
| 796.38 | 21.49 | QSSNRPAHNISHILGHDC[160]SSA | 192 | b6 |
| | | V | | |
| 796.73 | 21.54 | GPQGPTGSEGTPGLPGGVGQPG | 193 | b14 |
| | | AVGEK | | |
| 797.39 | 21.61 | QGTLSTAAPTTSPAPC[160]LSNH | 194 | b11 |
| | | HNK | | |
| 795.87 | 21.63 | ENSENTTAPEVFPR | 195 | b5 |
| 796.38 | 21.64 | EQQMVPGIPQGAHEA | 196 | y7 |
| 797.38 | 21.67 | LVAAC[160]PESC[160]VVC[160]TK | 197 | y2 |
| 795.88 | 21.89 | ASC[160]GQDQAAAETLLR | 198 | b10 |
| 796.37 | 21.9 | SMNDISLTPNTDQR | 199 | b6 |
| 796.71 | 21.91 | EDGTVSTANQNGVSSNGPGEILN | 200 | y6 |
| | | K | | |
| 796.88 | 22.14 | SNPEDQILYQTER | 201 | y2 |
| 796.39 | 22.23 | LEGMNETVSNLTQR | 202 | b5 |
| 796.38 | 22.3 | KPSSETDIENWASK | 203 | b7 |
| 796.88 | 22.31 | MSESLDTADPAVTGAK | 204 | y8 |
| 796.38 | 22.39 | VTLYEC[160]HSQGEIR | 205 | y14 |
| 797.41 | 22.43 | VHSHEVAAYLASPGR | 206 | b14 |
| 797.35 | 22.46 | AGC[160]QVVAPSDMMDGR | 207 | b14 |
| 796.37 | 22.52 | SISGTSTSEKPNSMDTANTSPFK | 208 | y6 |
| 795.89 | 22.6 | NFQSESVPALGGQEK | 209 | y6 |
| 797.36 | 22.64 | DSPVC[160]PSYSPTMPR | 210 | b14 |
| 797.42 | 22.71 | DRPSLPQERPGWR | 211 | b14 |
| 797.7 | 22.97 | EDGEVVQEEEVC[160]AKPSVTEE | 212 | y5 |
| | | K | | |
| 795.89 | 23 | DAETGEEVTHYLVK | 213 | b4 |
| 795.92 | 23.2 | TGSLPHSSEQLLGHK | 214 | b4 |
| 797.39 | 23.31 | ASSVLPEHHEAFNR | 215 | b14 |
| 797.68 | 23.41 | LSGAMC[160]TSC[160]ASQTTAND | 216 | b5 |
| | | PYTVR | | |
| 795.91 | 23.5 | VSTAQDVIQQTLC[160]K | 217 | b4 |
| 795.9 | 23.61 | VSSSSESEPELAQLK | 218 | b12 |
| 796.62 | 23.61 | TSC[160]SNC[160]TSNGMEC[160] | 219 | b12 |
| | | MWC[160]SSTK | | |
| 796.89 | 23.77 | ESSQPPVAFSSSIEK | 220 | y2 |
| 797.35 | 23.82 | NC[160]QTVLAPC[160]SPNPC[160] | 221 | b4 |
| | | ENAAVC[160]K | | |
| 797.36 | 23.83 | GQDTQFWAGHYGAR | 222 | b14 |
| 796.89 | 23.92 | MTENVVC[160]TGAVNAVK | 223 | b5 |
| 797.41 | 23.94 | YPSTSEAVNIQGISK | 224 | b6 |
| 795.9 | 24.04 | AGFPC[160]FKPSGAAPQR | 225 | b7 |
| 796.41 | 24.06 | STLEEQGLHVHSVR | 226 | b13 |
| 796.9 | 24.06 | GTANTC[160]IPSISSIGSK | 227 | y3 |
| 797.42 | 24.19 | SFNQNSHLIIHQR | 228 | b14 |
| 795.87 | 24.23 | IPIDNMTNEMEQR | 229 | b7 |
| 796.42 | 24.31 | INLPAPNPDHVGGYK | 230 | y2 |
| 796.93 | 24.48 | SPGTHLGALAQTVQGR | 231 | y10 |
| 797.04 | 24.48 | LSNC[160]DPPPTYEEATGQVNLQ | 232 | b6 |
| | | R | | |
| 795.87 | 24.76 | EDISAC[160]LQGTHGFR | 233 | b6 |
| 796.37 | 24.76 | EDC[160]C[160]PGKPLNVFR | 234 | y4 |
| 795.82 | 24.82 | EC[160]DMC[160]FSQASSLR | 235 | y4 |
| 795.84 | 24.91 | DC[160]AANTFIEDSGYK | 236 | y5 |
| 796.4 | 24.93 | LQESGQVTISELC[160]K | 237 | y10 |
| 795.88 | 24.95 | SQAC[160]GGNLGSIEELR | 238 | b6 |
| 797.08 | 25.01 | GPHPQALPGHLPGAGDSGAGAG | 239 | b9 |
| | | GGVVR | | |
| 795.89 | 25.08 | LEYSGAISAHC[160]NLR | 240 | b11 |
| 797.38 | 25.1 | MGVTC[160]VSQMPVAEGK | 241 | y12 |
| 797.33 | 25.14 | TC[160]DFFSPYENGEK | 242 | y14 |
| 796.84 | 25.16 | FGYYGDALQQDC[160]R | 243 | b12 |
| 797.41 | 25.26 | LLSLHSPNSYYGSR | 244 | b14 |
| 796.91 | 25.27 | YQVSEEVPSGTVIGK | 245 | y2 |
| 796.9 | 25.38 | ESTATLLGC[160]NASIQK | 246 | b4 |
| 796.9 | 25.48 | TSVTTSISEPWTQR | 247 | b13 |
| 795.83 | 25.52 | MSYSC[160]C[160]LPSLGC[160]R | 248 | y3 |
| 796.36 | 25.56 | SEYSSYPDINFNR | 249 | b4 |
| 795.91 | 25.71 | HSHGLALQPSFPGSR | 250 | b4 |
| 796.43 | 25.74 | LPYSGRPAPAPAAAPGV | 251 | b5 |
| 795.9 | 25.75 | AEQDITTLEQSISR | 252 | y4 |
| 796.89 | 25.83 | VMSPENFPTASVEGK | 253 | y3 |
| 796.43 | 25.99 | GGNQEIGPLPPTGNLK | 254 | y11 |
| 796.86 | 26.03 | GAEVTYMNMTAYNK | 255 | b10 |
| 797.47 | 26.05 | SPAARPPVPAPPALPR | 256 | b14 |
| 797.4 | 26.12 | C[160]LVPGYYSTHLQR | 257 | b14 |
| 797.45 | 26.16 | LAPISEEGKPQLVGR | 258 | b14 |
| 795.86 | 26.18 | DEDLQEMENLAQR | 259 | b7 |
| 795.89 | 26.26 | WIHFGTEVTNSSGR | 260 | y8 |
| 796.44 | 26.37 | VLPMVPAPPGSSAAAAR | 261 | y14 |
| 796.38 | 26.41 | SNFSPHFASSNQLR | 262 | y5 |
| 796.34 | 26.42 | C[160]LHC[160]LYSC[160]HWR | 263 | ¥9 |
| 795.9 | 26.47 | VGNIPYEATEEQLK | 264 | b7 |
| 797.36 | 26.48 | HPDEAAFFDTASTGK | 265 | y5 |
| 795.89 | 26.58 | SSLTQEEAPVSWEK | 266 | y4 |
| 796.92 | 26.58 | VLNQYTDTIIQER | 267 | y2 |
| 796.4 | 26.64 | LEQGENVFLQATDK | 268 | y7 |
| 796.87 | 26.64 | MAFMAATDHSNQLR | 269 | y5 |
| 796.7 | 26.7 | MNSPSQSSPGMNPGQPTSMLSPR | 270 | b4 |
| 796.93 | 26.9 | FVYPVPYTTRPPR | 271 | y2 |
| 797.36 | 26.94 | WDSNIC[160]ELHYTR | 272 | b14 |
| 796.39 | 27.02 | YPAEEPASAWTPSPPPVTTSSSK | 273 | b8 |
| 795.89 | 27.07 | TSANPETLLGEMEAK | 274 | y2 |
| 796.4 | 27.15 | MTQPFPTQFAPQAK | 275 | y10 |
| 796.89 | 27.18 | TFSQMSSLVYHHR | 276 | y4 |
| 795.91 | 27.21 | VGQC[160]VVVFSQAPSGR | 277 | y12 |
| 797.41 | 27.35 | NPSTNVSVVVFDSTK | 278 | b6 |
| 795.9 | 27.37 | VLMVETHNEIYDK | 279 | b5 |
| 795.87 | 27.41 | MDNC[160]LAAAALNGVDR | 280 | b6 |
| 796.4 | 27.45 | LVIEC[160]GADC[160]NILSK | 281 | b5 |
| 795.85 | 27.52 | FC[160]C[160]EDGTTIVNFK | 282 | y7 |
| 796.41 | 27.55 | YHYAEISSQVPLGK | 283 | y4 |
| 797.43 | 27.55 | LQTLSIQQC[160]LPHR | 284 | b14 |
| 795.91 | 27.57 | LVMVSTLDTSSQPGR | 285 | y10 |
| 796.44 | 27.62 | QPPPGIVAPAAMLSSR | 286 | y5 |
| 796.94 | 27.63 | SPLQAVEPISTSVHK | 287 | y7 |
| 797.4 | 27.8 | IVETDESQGIFVEK | 288 | y3 |
| 795.9 | 27.92 | LSWPQSTGIC[160]SNIK | 289 | y4 |
| 795.91 | 27.97 | SVLDLGSGC[160]GATAIAAK | 290 | b5 |
| 796.88 | 28.05 | AAGFDEIEQDLTQR | 291 | b13 |
| 796.89 | 28.06 | QTVMTSATWPDTVR | 292 | b5 |
| 797.4 | 28.07 | TAFTNHQIYELEK | 293 | y14 |
| 797.44 | 28.17 | APSSPALQALAGQAGVR | 294 | b14 |
| 796.93 | 28.21 | MEPAVGGPGPLIVNNK | 295 | y11 |
| 796.94 | 28.21 | QVHILQQNC[160]IALR | 296 | b4 |
| 796.36 | 28.31 | VEVSGDASC[160]C[160]SPDPISPE | 297 | y11 |
| | | DLPR | | |
| 797.39 | 28.33 | EGFPTDAPYPTTLGK | 298 | b4 |
| 796.9 | 28.4 | SAYALGGLGSGIC[160]PNR | 299 | b4 |
| 796.9 | 28.51 | ASSLC[160]HHASLPWVK | 300 | b4 |
| 797.38 | 28.69 | FLNDTSLPHSC[160]FR | 301 | b14 |
| 796.92 | 28.7 | FLIDSNGQVITTER | 302 | y2 |
| 796.41 | 28.77 | IEGENYLPQPIYR | 303 | y3 |
| 796.89 | 28.89 | VTGLIENHDYEFR | 304 | y3 |
| 796.43 | 28.9 | ILFVSQGSEIASQGR | 305 | b6 |
| 797.42 | 28.93 | DLDQVQLHLEEVR | 306 | b14 |
| 796.87 | 28.98 | FNDITADVYSEYR | 307 | b4 |
| 796.95 | 29.03 | LC[160]VPGIVALQSPPNK | 308 | y11 |
| 797.07 | 29.36 | IEETC[160]QVGMKPPVPGGYTLQ | 309 | b11 |
| | | GK | | |
| 797.42 | 29.42 | VWPQATAPEQAPAPARPYQGVR | 310 | y5 |
| 796.9 | 29.54 | LGVEMLSESQLSDGK | 311 | y8 |
| 797.72 | 29.55 | QNEHHLEGGFSIGSVGPDGQLGR | 312 | y5 |
| 796.39 | 29.62 | NFLETDNEGNGILR | 313 | y10 |
| 796.41 | 29.64 | GILFVGSGVSGGEEGAR | 314 | b5 |
| 797.41 | 29.69 | VTDATETTITISWR | 315 | b14 |
| 796.37 | 29.81 | EWDILETEEHYK | 316 | b6 |
| 796.72 | 29.88 | QVRPPSC[160]PVPFPETFNGESSR | 317 | y9 |
| 796.94 | 29.96 | VVFHLHESFPRPK | 318 | b7 |
| 795.91 | 30 | MYQGHMQVVGVTLK | 319 | b5 |
| 796.03 | 30.05 | SPGDGGPHDVFTSLPSDC[160]QL | 320 | b5 |
| | | GSR | | |
| 797.42 | 30.22 | WAAGAMAAPEPLRPR | 321 | b14 |
| 796.89 | 30.25 | AQLEEEIIAYEER | 322 | y2 |
| 796.87 | 30.39 | MSEDSSALPWSINR | 323 | b8 |
| 796.84 | 30.46 | LMNEDPMYSMYAK | 324 | b4 |
| 797.41 | 30.55 | GLGAFVIDSDHLGHR | 325 | b14 |
| 795.92 | 30.6 | ADANTAAIQAVLYNR | 326 | y4 |
| 797.39 | 30.69 | EDIVTEQIDFSAAR | 327 | b14 |
| 796.71 | 30.71 | MVRPQDTVAYEDLSEDYTQK | 328 | b13 |
| 796.87 | 30.72 | DYFQHPHFSTWK | 329 | y3 |
| 796.4 | 30.73 | IVESMQSTLDAEIR | 330 | b4 |
| 796.7 | 30.74 | SLSNENYGVYNC[160]SIINEAGAG | 331 | y3 |
| | | R | | |
| 796.38 | 30.75 | QSWNPFPDFTPQK | 332 | b10 |
| 797.71 | 30.87 | LTWPTDAGPDDAAVDTSSEITTK | 333 | y9 |

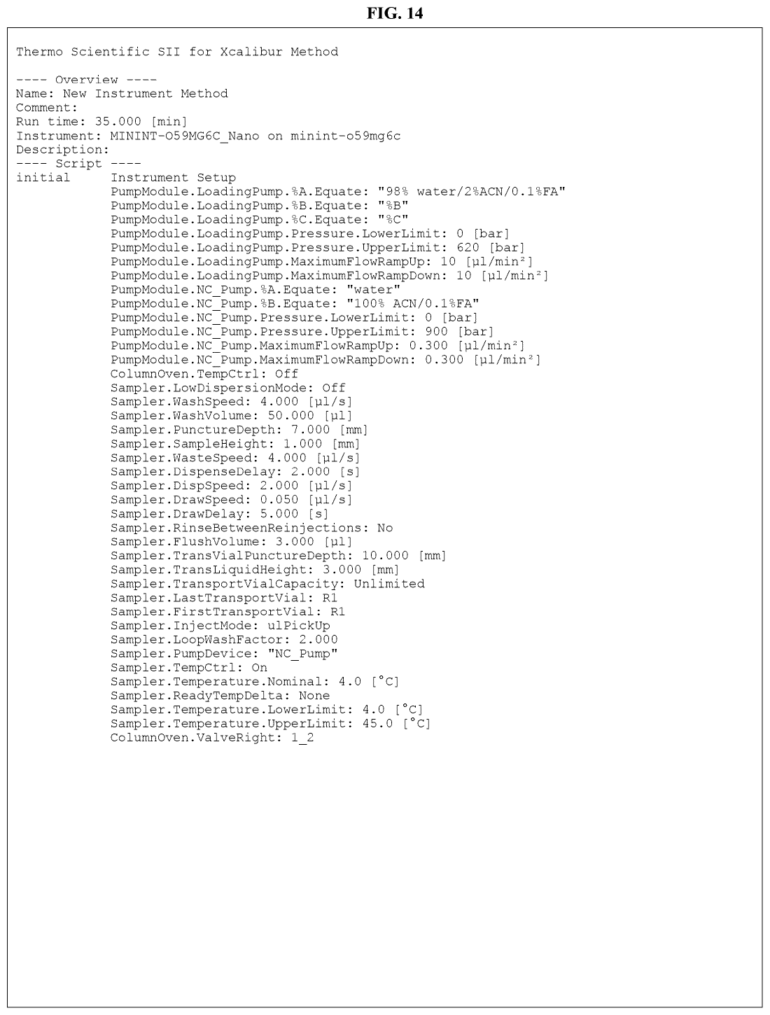

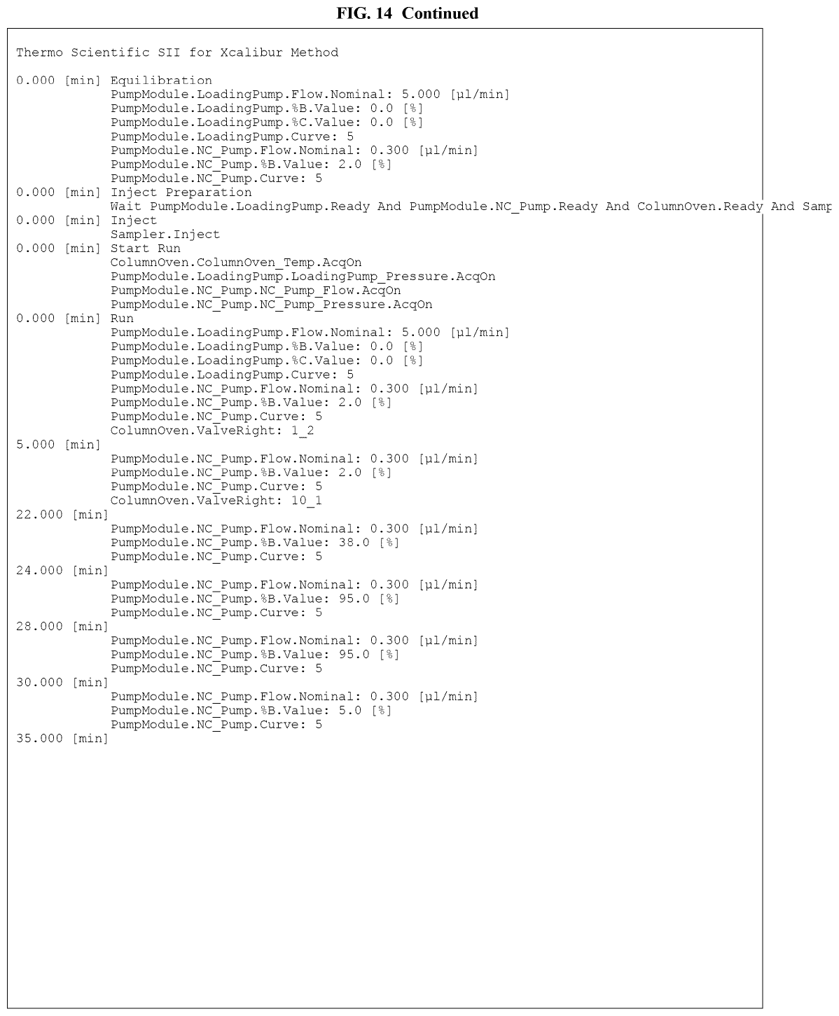

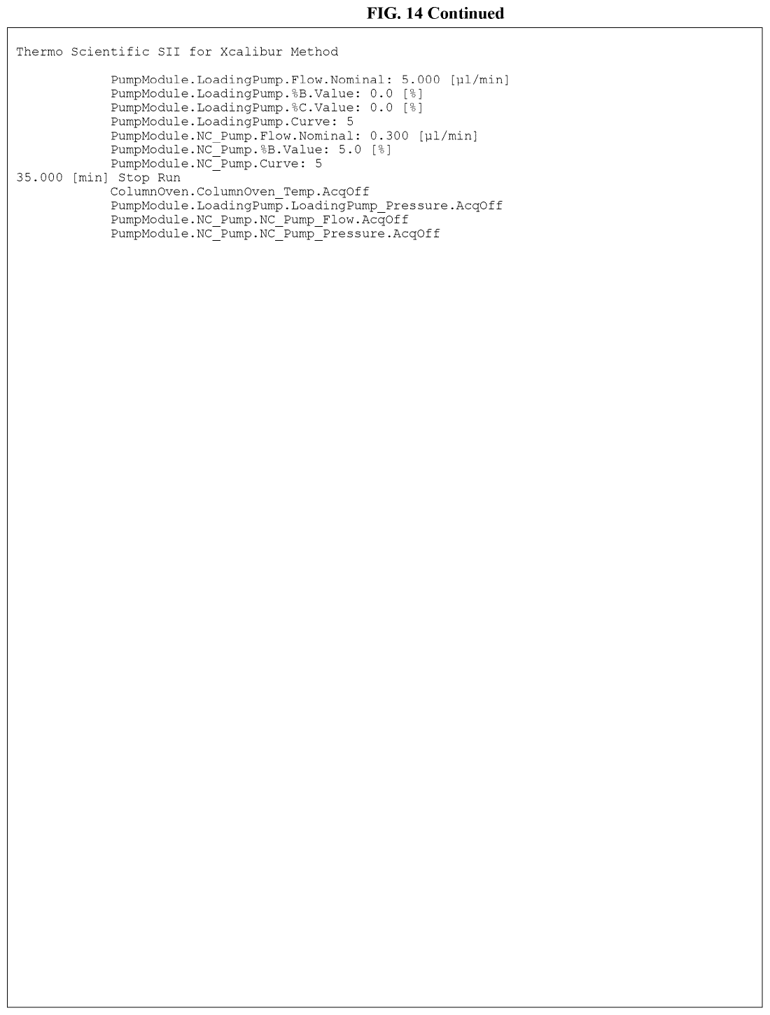

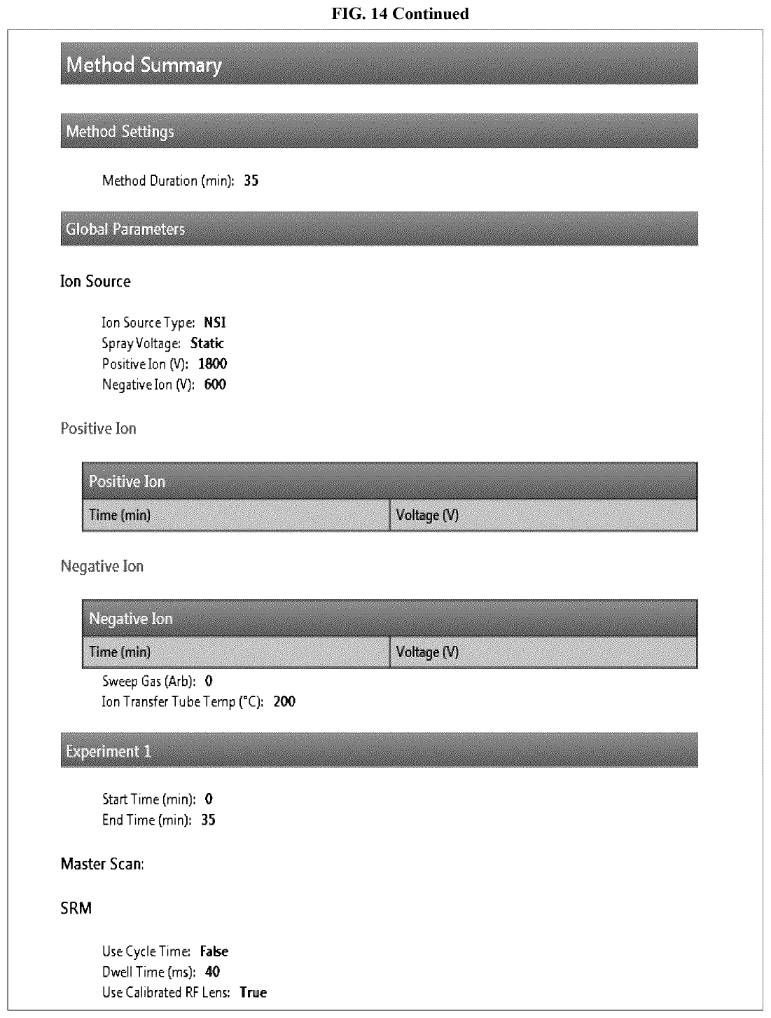

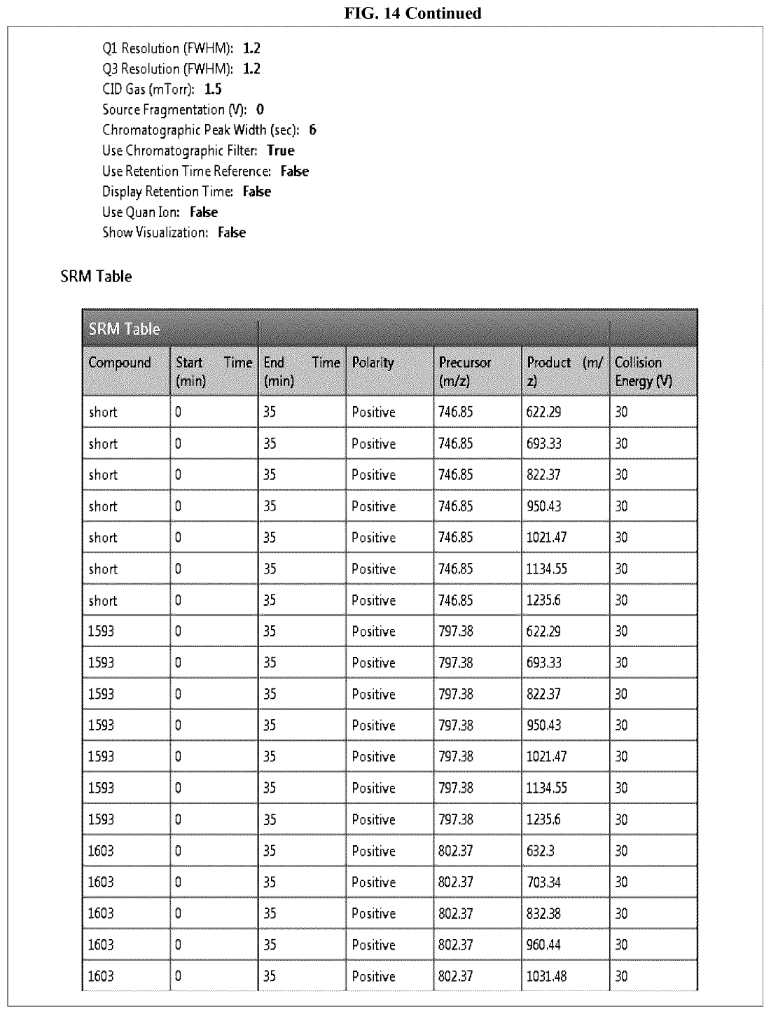

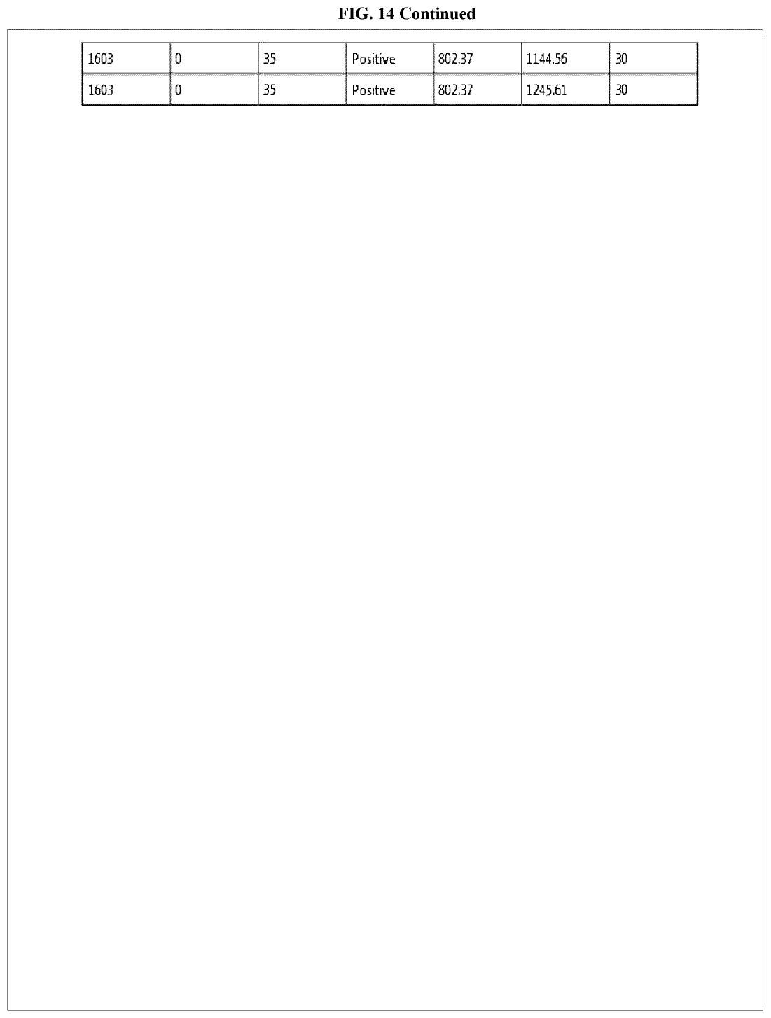

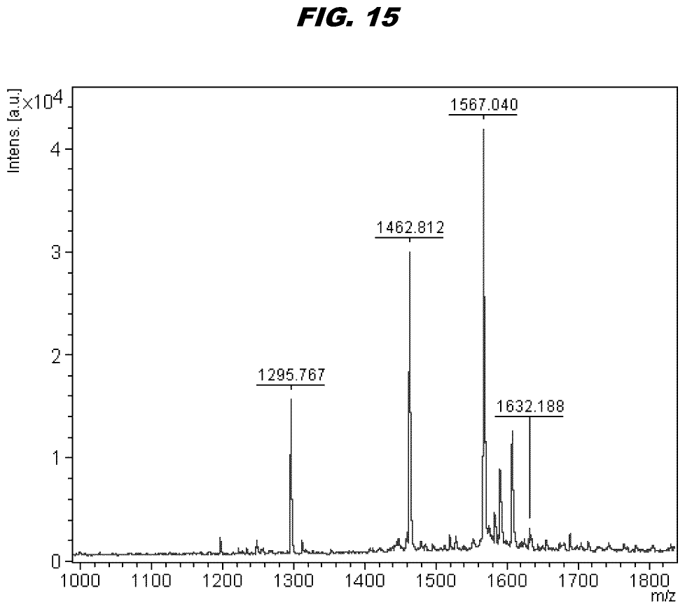

FIG. 14 presents the LC-MRM instrument method.

### Example 3-LC-PRM-MS Based Diagnostic Methods and Proteotypic Peptide Biomarkers Specific to HIV-1/2 p24 Antigen

Human immunodeficiency virus (HIV) testing and counseling is an essential first step in controlling the virus, but the World Health Organization (WHO) reports that only about half of people currently infected with HIV worldwide are aware of their status. Even though the chief rationale for the situations may include socioeconomic factors associated with HIV, such as poverty, the low level of population's knowledge and attitudes about HIV infections, as well as the patient's behaviors, the diagnostic and treatment monitoring challenges in patients with HIV infection, especially those also infected with Tuberculosis (TB), are major contributors for the missed or delayed of HIV diagnosis or treatment.

Currently, the diagnosis of HIV seems to be much straightforward because of the availability of rapid diagnostic tests (RDTs), Ab/Ag Combination Assays (ELISA) and Nucleic Acid Amplification Test (NAT) for HIV infection.

However, the low sensitivity and specificity of RDTs remains to be a major concern, even though it is fast and cost effective ($2.0 per test) for low resource setting.

For Ab/Ag Combination Assays, further validation of the results by western blot and extensive performance evaluation are still needed for Ab/Ag Combination Assay in diverse field settings, even though they are more sensitive and specific than RDTs and with low requirement on equipment and operator. Moreover, due to the presence of antigen-specific host antibodies and immunoglobulin-specific, rheumatoid-factor-like antibodies, as well as the existence of homologs of target antigen proteins, Ab/Ag Combination assay that designed to test the intact antigen protein was confronted with several problems such as false positives, false negatives and other interferences.

More importantly, both of RDTs and Ab/Ag Combination assay are not suitable for infant (<18 months) HIV screening (due to the presence of maternal antibodies) or antiretroviral treatment (ART) monitoring, since the RDTs are mainly designed to test for HIV antibodies, and most of the currently available Ab/Ag combination assays gave only a single result and did not differentiate whether a positive result was due to the presence of the HIV-1 p24 antigen or due to the presence of antibody to HIV-1 or-2. Therefore, infant HIV screening or the standard of care to monitor ART are typically achieved by NAT based on plasma HIV RNA concentration.

However, the NAT is a highly technically demanding, involving the issues of high costs ($160.07 per test), dedicated infrastructure facility, equipment, consumables and technical expertise. Moreover, most of the commercially marketed NAAT test for HIV are monoplex-PCRs, which are bound to miss out the detection of both HIV-1 and HIV-2 in a single NAT test. Furthermore, no commercial NAT test for HIV-2 are available based on our best knowledge.

To address the issues, an LC-iSPRM-MS diagnostic platform for single step, rapid and quantitative detection of HIV-1 and HIV-2 antigens with multiple enhancement of sensitivity and specificity was developed.

We identified a panel of new proteotypic peptide biomarkers specific to HIV-1/2 p24 antigen and developed an LC-iSPRM-MS diagnostic platform for single assay, rapid and quantitative detection of HIV-1 and HIV-2 antigens with multiple enhancement of sensitivity and specificity. LC-iSPRM-MS can not only sensitively and specifically identify and measure pathogen burden directly from clinical samples, including serum, plasma, or whole blood obtained from a human, but also provide one step confirmation and discrimination of HIV-1 or HIV-2 infections based on peptide sequence, and further allows assess treatment response in patients infected with HIV-1 or HIV-2. The detection methods include but not limited to mass spectrometry, chromatography, electrochemistry and chemical sensors, electrophoresis, immunochemical techniques, nanotechnology and microfabrication, and dipstick point of care assays.

### Pretreatment of Serum Plasma

Serum samples (50 μL) were added with 450 μL of Viral Lysis Buffer (0.4% TritonX-100, 0.2% SDS in PBS, pH=7.4), heating at 100° C. for 5 min, then cooling down the temperature into room temperature. Add 10 μl IM Tris.

Microwave digested (P4 level microwave power, 6*5 min) with 10 μg (25 μl) sequence grade Trypsin (sequence grade modified, Promega, more trypsin may be needed (40-50 μg) if aggregation appeared), then 37° C. for 1 h.

### Protein G-Dynabeads Coupling

Prepare protein G Dynabeads®: Resuspend Dynabeads® in the vial (vortex >30 sec or tilt and rotate 5 min). Transfer 100 μL (3 mg) Dynabeads® to a tube. Place the tube on the magnet to separate the beads from the solution and remove the supernatant. Wash the beads once with 400 uL of PBS with 0.2% Tween®-20 and remove the supernatant from the beads.

Binding of Antibody: Add monoclonal HIV-1/2 antibody (Ab) (50 μg) diluted in 400 uL PBS with 0.2% Tween®-20, to the Dynabeads® from above. Incubate with rotation for 1h at room temperature. Place the tube on the magnet and wash the beads twice with 400 μL PBS with Tween®-20. Remove the tube from the magnet and resuspend the beads-Ab complex in 400 μL PBS with Tween®-20. Wash by gentle pipetting.

### Immunoprecipitate Target Antigen

After added TFA to a final concentration of 0.1% (5 μL), the digested serum samples were spiked with 1.0 μL of 50 nmol/L stable isotope labeled internal standard peptide. Added 25 μL of HIV-1/2 antibody conjugated beads into each serum samples. For each sample, the antibody is 50/16=3.1 μg, and the beads is 3/16=0.1875 mg. Incubate with rotation for 1 hour at room temperature. Place the tube on the magnet and remove the supernatant. Wash the beads-Ab complex 2 times with 400 μl PBS, and once with 100 μL of LC grade water. Place the tube on the magnet and remove the supernatant. Add 20 μL Elution Buffer (1% Formic Acid) and incubated at RT for 15 min at HulaMixer. Place the tube on the magnet and transfer the supernatant containing eluted Ab and Ag to an Eppendorf LoBind tube. Transfer 16 μL of the supernatant to sample vial for LC-MS analysis (15 μL injection).

### LC-PRM-MS Analysis

Eluates were loaded on a C18 trap column, eluted onto a C18 analytical column, and fractionated with a 0.3 μL/min acetonitrile/formic acid gradient (5-40%) and analyzed using the PRM Mode on a nano-LC UltiMate 3000 high performance liquid chromatography (HPLC) system coupled with an Orbitrap Fusion™ Lumos™ Tribrid™ Mass Spectrometer (Thermo Fisher Scientific). Skyline software version 4.1.0.18169 (MacCoss Lab Software) was used to analyze serum MS and MS/MS spectra against a library produced using recombinant p24 and CFP-10 digests.

### Example 4—HIV Peptide Alignment

FIG. 15 shows a mass spectrum of a trypsin digest of recombinant p24, which generated only three major peaks that matched those provided by a bioinformatic analysis of this sequence (Table 4).

TABLE 4
List of [M + H]⁺ values for p24 trypic peptides provided by Skyline
analysis (table discloses SEQ ID NOS 334, 5, 335, 7, and 336,
respectively, in order of appearance).

| [M + H]⁺ | SEQUENCE | Peptide ID | Sequence variation within the HIV-1 M group |
|---|---|---|---|
| 1566.8321 | VHPVHAGPIAPGQMR | (PEPTIDE2) | [6 Class M variant aa positions; 6/15: 40.0%] |
| 1462.6444 | ETINEEAAEWDR | (PEPTIDE1) | [2 Class M variant aa positions; 2/12: 16.6%] |
| 1356.6430 | PEPTAPPEESFR | (ND) | |
| 1295.6664 | MYSPTSILDIR | (PEPTIDE3) | [3 Class M variant aa positions; 3/11: 27.2%] |
| 1144.5844 | SGVETTTPPQK | (ND) | |

Peptide IDs were assigned based on their order of appearance in the p24 aa sequence. Variable aa positions in the the p24 sequence of HIV-1 group M viruses were identified after alinging all reviewed p24 gag entries in the UniProtKB virus database (January 2020).

All the HIV p24 sequences found in the UniProtKB database were aligned that had reviewed entries and identified two peptides regions that were completely conserved among the aligned sequences of all these p24 entries (GSDIAGTTS (SEQ ID NO: 337) and QGPKEPFRDYVDRF (SEQ ID NO: 338)). In addition, the alignments of all three Skyline entries with matching tryptic peaks were obtained.

A search of the UniProtKB database with the two completely conserved peptides gave an estimate of the total number of UniProtKB HIV-1 entries that contained this region (Table 5:˜60,000), which were subsequently used to estimate the relative frequency of the amino acid variants of our target peptides among all HIV-1 p24 entries with the corresponding sequence.

Table 5. Estimate of HIV-1 gag sequence entries in the UniProKB database that contain the target peptide regions. Table discloses SEQ ID NOS 337 and 338, respectively, in order of appearance.

### AA SEQUENCE HIV-1 ENTRIES TOTAL UNIPROTKB ENTRIES

GSDIAGTTS ˜60,829 [62,996 total: including 48 HIV2+2,119 SIV]
QGPKEPFRDYVDRF 60,443 [no HIV-2 or SIV]

Alignment of all reviewed HIV-1 p24 entries in the UniProtKB virus database (January 2020) identified two aa sequence regions adjacent to the target peptides that were completely conserved among all HIV-1 p24 entries: GSDIAGTTS (SEQ ID NO: 337) and QGPKEPFRDYVDRF (SEQ ID NO: 338). Peptide searches of the UniProtKB virus database were performed to identify the number of entries that matched these sequences and entries that did not have HIV-1 identifiers were excluded from the estimate of HIV-1 p24 entries.

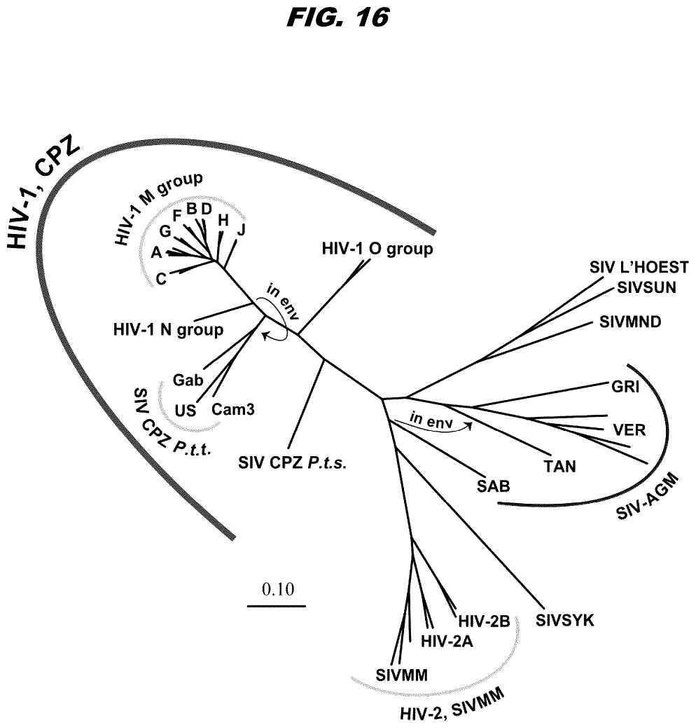

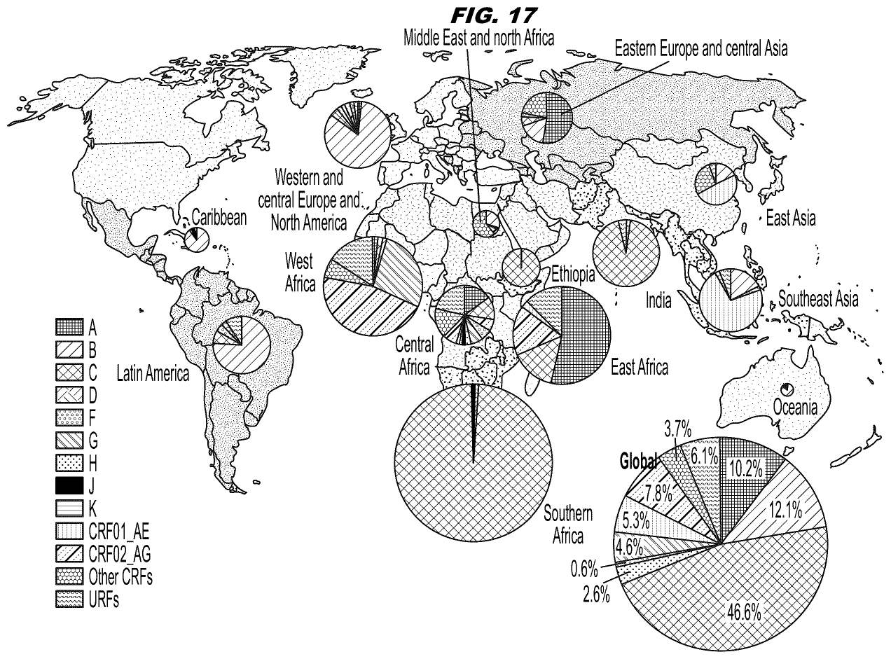

Table 6 depicts the alignment of all three target peptides according to their species, group, subtype and viral isolate, where the evolutionary relationship amongst these groups is shown in FIG. 16. Note that Group M is estimated to account for >90% of HIV/AIDS cases, with the breakdown of the contribution of individual HIV-1 groups shown in FIG. 17.

TABLE 6
Alignment of PEPTIDE1, PEPTIDE2 and PEPTIDE3 in reviewed HIV-
1/2 p24 and SIV p247 sequences found in the UniproKB virus database. Table 6 discloses
“PEPTIDE1”, “PEPTIDE2”, and “PEPTIDE3” sequences as SEQ ID NOS 349-540,
respectively, in order of appearance. Table discloses “PEPTIDE1 and PEPTIDE2”
sequences as SEQ ID NOS 541-604, respectively, in order of appearance.

| ENTRY | PEPTIDE1 (1462) PEPTIDE2 (1566) | PEPTIDE3 (1295) | GROUP: SUBTYPE (ISOLATE) |
|---|---|---|---|
| | MQMLKDTINEEAADWDR | VHPVHAGPIPPGQMREPRGS | NKIVRMYSPVSILDIRQGPKE |
| | M:A (MAL) | | |
| P24740 | MQMLKDTINEEAAEWDR | LHPVHAGPIPPGQMREPRGS | NKIVRMYSPVSILDIRQGPKE |
| | M:A (U455) | | |
| Q73367 | MQMLKETINEEAAEWDR | LHPVQAGPVAPGQMREPRGS | NKIVRMYSPSSILDIKQGPKE |
| | M:B (89.6) | | |
| O12158 | MQMLKDTINEEAAEWDR | LHPVHAGPVAPGQMREPRGS | NKIVRMYSPVSILDIKQGPKE |
| | M:B (92BR025) | | |
| P03349 | MQMLKETINEEAAEWDR | VHPVHAGPIAPGQMREPRGS | NKIVRMYSPTSILDIRQGPKE |
| | M:B (ARV2/SF2) | | |
| P03347 | MQMLKETINEEAAEWDR | VHPVHAGPIAPGQMREPRGS | NKIVRMYSPTSILDIRQGPKE |
| | M:B (BH10) | | |
| P04593 | MQMLKETINEEAAEWDR | VHPVHAGPIAPGQMREPRGS | NKIVRMYSPTSILDIRQGPKE |
| | M:B (BH5) | | |
| P03348 | MQMLKETINEEAAEWDR | VHPVHAGPIAPGQMREPRGS | NKIVRMYSPTSILDIRQGPKE |
| | M:B (BRU/LAI) | | |
| P05887 | MQMLKETINEEAAEWDR | LHPVHAGPIAPGQMREPRGS | NKIVRMYSPISILDIRQGPKE |
| | M:B (CDC-451) | | |
| Q75001 | MQMLKDTINEEAAEWDR | LHPVHAGPVAPGQMRDPRGS | NKIVRMYSPVSILDIKQGPKE |
| | M:B (ETH2220) | | |
| P04591 | MQMLKETINEEAAEWDR | VHPVHAGPIAPGQMREPRGS | NKIVRMYSPTSILDIRQGPKE |
| | M:B (HXB2) | | |
| P12494 | MQMLKETINEEAAEWDR | LHPAQAGPIAPGQMREPRGS | NKIVRMYSPSSILDIRQGPKE |
| | M:B (JH32) | | |
| P20873 | MQMLKETINEEAAEWDR | LHPVHAGPIAPGQMREPRGS | NKIVRMYSPVSILDIRQGPKE |
| | M:B (JRCSF) | | |
| Q70622 | MQMLKETINEEAAEWDR | VHPVHAGPIAPGQMREPRGS | NKIVRMYSPTSILDIRQGPKE |
| | M:B (LW123) | | |
| P05888 | MQMLKETINEEAAEWDR | LHPAHAGPIAPGQMREPRGS | NKIVRMYSPSSILDIRQGPKE |
| | M:B (MN) | | |
| P12493 | MQMLKETINEEAAEWDR | LHPVHAGPIAPGQMREPRGS | NKIVRMYSPTSILDIRQGPKE |
| | M:B (NY5) | | |
| P20889 | MQMLKETINEEAAEWDR | LHPVHAGPIAPGQMREPRGS | NKIVRMYSPTSILDIRQGPKE |
| | M:B (OYI) | | |
| P05890 | MQMLKETINEEAAEWDR | LHPVHAGPIAPGQMREPRGS | NKIVRMYSPISILDIRQGPKE |
| | M:B (RF/HAT3) | | |
| P24736 | MQMLKDTINEEAAEWDR | LHPVHAGPIPPGQMREPRGS | NKIVRMYSPVSILDIRQGPKE |
| | M:B (U455) | | |
| P05889 | MQMLKETINEEAAEWDR | LHPVHAGPIAPGQMREPRGS | NKIVRMYSPTSILDIRQGPKE |
| | M:B (WMJ22) | | |
| P35962 | MQMLKETINEEAAEWDR | LHPVHAGPIAPGQMREPRGS | NKIVRMYSPTSILDIRQGPKE |
| | M:B (YU-2) | | |
| O12157 | MQMLKDTINEEAAEWDR | LHPVHAGPVAPGQMREPRGS | NKIVRMYSPVSILDIKQGPKE |
| | M:C (92BR025) | | |
| Q75002 | MQMLKDTINEEAAEWDR | LHPVHAGPVAPGQMRDPRGS | NKIVRMYSPVSILDIKQGPKE |
| | M:C (ETH2220) | | |
| P04592 | MQMLKETINEEAAEWDR | LHPVHAGPIAPGQMREPRGS | NKIVRMYSPVSILDIRQGPKE |
| | M:D (ELI) | | |
| P18800 | MQMLKETINDEAAEWDR | LHPVHAGPVAPGQMREPRGS | NKIVRMYSPVSILDIRQGPKE |
| | M:D (NDK) | | |
| P12495 | MQMLKETINEEAAEWDR | LHPVHAGPIAPGQMREPRGS | NKIVRMYSPVSILDIRQGPKE |
| | M:D (Z2/CDC-Z34) | | |
| O89291 | MQMLKDTINEEAAEWDR | LHPTQAGPIPPGQIREPRGS | NKIVRMYSPVGILDIRQGPKE |
| | M:F1(93BR020) | | |
| Q9QSR4 | MQMLKDTINEEAAEWDR | LHPVHAGPAPPGQMREPRGS | NKIVRMYSPVSILDIKQGPKE |
| | M:F1(VI850) | | |
| Q9QBZ6 | MQMLKDTINEEAAEWDR | LHPVHAGPIPPGQMREPRGS | NKIVRMYSPVSILDIKQGPKE |
| | M:F2(MP255) | | |
| Q9QBZ2 | MQMLKDTINEEAAEWDR | LHPVHAGPIPPGQMREPRGS | NKIVRMYSPVSILDIKQGPKE |
| | M:F2(MP257) | | |
| POC1K7 | MQMLKDTINDEAAEWDR | IHPQQAGPIPPGQIREPSGS | NKIVRMYSPVSILDIRQGPKE |
| | M:G (92NG083) | | |
| O89939 | MQMLKDTINEEAAEWDR | MHPQQAGPFPPGQIREPRGS | NKIVRMYSPVSILDIRQGPKE |
| | M:G (SE6165) | | |
| O93182 | MQMLKDTINEEAAEWDR | VHPVHAGPIPPGQMREPRGS | NKIVRMYSPVSILDIKQGPKE |
| | M:H (90CF056) | | |
| Q9Q721 | MQMLKDTINEEAAEWDR | LHPVHAGPIPPGQMREPRGS | NKIVRMYSPVSILDIKQGPKE |
| | M:H (VI991) | | |
| Q9WC62 | MQMLKDTINEEAAEWDR | VHPVHAGPVAPGQVREPRGS | NKIVRMYSPVSILDIRQGPKE |
| | M:J (SE9173) | | |
| Q9WC53 | MQMLKDTINEEAAEWDR | VHPVHAGPIAPGQVREPRGS | NKIVRMYSPVSILDIRQGPKE |
| | M:J (SE9280) | | |
| Q9QBY4 | MQMLKDTINDEAAEWDR | LHPVHAGPIPPGQMREPRGS | NKIVRMYSPVSILDIRQGPKE |
| | M:K (96CM-MP535) | | |
| Q9QC00 | MQMLKDTINEEAAEWDR | MHPVQAGPIPPGQIREPRGS | NKIVRMYSPVSILDIRQGPKE |
| | M:K (97ZR-EQTB11) | | |
| Q9IDV8 | MQMLKEVINEEAAEWDR | THPAPVGPLPPGQMRDPRGS | NRIVRMYSPVSILEIKQGPKE |
| | N (YBF106) | | |
| O91079 | MQMLKEVINEEAADWDR | THPVPVGPLPPGQLRDPRGS | NRIVRMYSPVSILEIKQGPKE |
| Q77372 | LQVLKEVINEEAVEWDR | THPPPVGPLPPGQIREPTGS | NKMVKMYSPVSILDIKQGPKE |
| | O (ANT70) | | |
| Q79665 | LQVLKEVINEEAAEWDR | THPPAMGPLPPGQIREPTGS | NKMVKMYSPVSILDIRQGPKE |
| | O (MVP5180) | | |
| HIV2 | PEPTIDE1* (PEPTIDE1-PEPTIDE2) | PEPTIDE3 | GROUP: SUBTYPE (ISOLATE) |
| P24106 | MQIIREIINEEAADWDA-NHPIP-GPLPAGQLRDPRGS | QKCVRMYNPTNILDIKQGPKE | A (isolate CAM2) |
| P04584 | MQIIREIINEEAAEWDV-QHPIP-GPLPAGQLREPRGS | QKCVRMYNPTNILDIKQGPKE | A (isolate ROD) |
| Q74119 | MQIIREIINEEAADWDV-QHPIP-GPLPAGQLREPRGS | QKCVRMYNPTNILDVKQGPKE | A (isolate KR) |
| P20874 | MQIIREIINEEAADWDA-QHPIP-GPLPAGQLREPRGS | QKCVRMYNPTNILDIKQGPKE | A (isolate ST) |
| P17756 | MQIIREIINEEAADWDA-QHPIP-GPLPAGQLRDPRGS | QKCVRMYNPTNILDVKQGPKE | A (isolate D194) |
| P18095 | MQIIREIINEEAADWDS-QHPIP-GPLPAGQLRDPRGS | QKCVRKYNPTNILDIKQGPKE | A (isolate BEN) |
| P18041 | MQIIREIINDEAADWDA-QHPIP-GPLPAGQLRDPRGS | QKCVRMYNPTNILDVKQGPKE | A (isolate Ghana-1) |
| P05891 | MQIIREIINEEAADWDV-AHPIP-GPLPAGQLREPRGS | QKCVRMYNPTNILDINQGPKE | A (isolate NIH-Z) |
| P12450 | MQIIREIINEEAADWDV-QHPIP-GPLPAGQLRDPRGS | QKCVRMYNPTNILDIKQGPKE | A (isolate SBLISY) |
| Q76633 | MQIIREIINEEAADWDQ-QHPIP-GPLPAGQLRDPRGS | QKCVRMYNPTNILDIKQGPKE | B (isolate UC1) |
| P15832 | MQIIREIINEEAADWDQ-QHPSP-GPMPAGQLRDPRGS | QKCVRMYNPTNILDIKQGPKE | B (isolate D205) |
| Q74230 | MQIIREIINEEAADWDQ-QHPSP-GPMPAGQLREPRGS | QKCVRMYNPTNILDIKQGPKE | B (isolate EHO) |
| SIV2 | PEPTIDE1* (PEPTIDE1-PEPTIDE2) | PEPTIDE3 | GROUP: SUBTYPE (ISOLATE) |
| P12496 | MQIIREIINEEAADWDL-QHPQP-GPLPAGQLREPRGS | QKCVRMYNPTNILDVKQGPKE | SIV-mm (isolate F236/smH4) |
| P19504 | MQIIREIINEEAADWDL-QHPQP-GPIPPGQLREPRGS | QKCVRMYNPTNILDVKQGPKE | SIV-mm (isolate PBj14/BCL-3) |
| KU892415.1 | MQIIRDIINEEAADWDL-QHPQP-APQ-QGQLREPSGS | QKCVRMYNPTNILDVKQGPKE | SIV-mac (251/32H/L28) |
| D01065.1 | MQIIRDIINEEAADWDL-QHPQP-APQ-QGQLREPSGS | QKCVRMYNPTNILDVKQGPKE | SIV-mac (32H) |
| KF051800.1 | MQIIRDIINEEAADWDL-QHPQP-APQ-QGQLREPSGS | QKCVRMYNPTNILDVKQGPKE | SIV-mac (251) |
| M33262.1 | MQIIRDIINEEAADWDL-QHPQP-APQ-QGQLREPSGS | QKCVRMYNPTNILDVKQGPKE | SIV-mac (239) |
| P05894 | MQIIRDIINEEAADWDL-QHPQQ-APQ-QGQLREPSGS | QKCVRMYNPTNILDVKQGPKE | SIV-mac (Mm142-83) |
| P05897 | MQIIRDIINEEAADWDL-QHPQP-APQ-QGQLREPSGS | QKCVRMYNPTNILDVKQGPKE | SIV-mac (K6W) |
| P05893 | MQIIRDIINEEAADWDL-QHPQP-APQ-QGQLREPSGS | RLQKCVYNPINILDVKQRPKE | SIV-mac (K6W) |
| P31634 | MQIIREIINEEAADWDV-QHPQP-GPLPAGQLREPSGS | QKCVRMYNPVNILDIKQGPKE | SIV-mac (STM) |

Target peptides are presented with five aa of flanking sequence except where one target peptide abuts another (PEPTIDE1 and PEPTIDE2). A single entry is shown for each isolate. Underlining and bold formatting indicate the highly conserved (bold) and variable (underlined) aa's in each target peptide. Dashes indicate spaces introduced to promote aa sequence alignment.

Table 6 indicates that PEPTIDE1 (ETINEEAAEWDR (SEQ ID NO: 5)) is highly conserved amongst all HIV-1 groups, varying at a single residue (position 1) in all but one of the group M sequence isolates [HIV-1 M: A (MAL)], with both variant positions consisting of conservative D to E substitutions. Additional variants, including one at the same position, are present in individual group N and O isolates, but these minor HIV-1 groups are rare and restricted to very limited geographical areas.

PEPTIDE2 (VHPVHAGPIAPGQMR (SEQ ID NO: 334)) contains multiple sites of amino acid variation spread throughout its length and demonstrates significant variation even within the group M subtypes. Multiple antibodies would be required to capture all the variants of this peptide, making a poor biomarker target.

PEPTIDE1 and PEPTIDE2 would not be generated in HIV-2 or SIV due to a variable amino acid substitution that removes the R reside required to separate these two peptides. An antibody generated to PEPTIDE1, but not PEPTIDE2, might be able to capture this fusion peptide, but it should demonstrate a significantly different elution profile and a would have a much higher mass to charge ratio than PEPTIDE1 isolates from any HIV-1 group and subtype.

PEPTIDE3 (MYSPTSILDIR (SEQ ID NO: 7)) was less conserved than PEPTIDE1 but most of its amino acid variation was restricted to two positions (residues 5 and 11). Most of the HIV-1 group and subtypes, except HIV-1 group M subtype B, had a V residue at position 5 of PEPTIDE3, while a conservative K or R variation at residue 11 was almost equally splits between the various group M subtypes.

PEPTIDEI antibodies were generated using ETINEEAAEWDR (SEQ ID NO: 5) as the immunogen and they also efficiently detected the DTINEEAAEWDR (SEQ ID NO: 6) peptide. This seems to be a safe choice, particularly since the only other variation was much rarer and was also a conservative E to D substitution. Together these two variants accounted for 96.8% of all the HIV-1 p24 sequences present in the UniProtKB database, implying that we should be able to detect p24 peptides from most groups and subtypes, although this analysis is subject to selection bias in that the deposited sequences may not accurately represent the global distribution of HIV-1 p24 sequence variation.

PEPTIDE3 antibodies were generated using MYSPTSILDIR (SEQ ID NO: 7) as the immunogen and these antibodies efficiently detected the MYSPVSILDIK (SEQ ID NO: 8) peptide, although their performance with any of the other group M subtype B sequence variants was not examined. Due to the sequence variation in group M subtype B at this position, it is possible that these antibodies could have reduced affinity of some of these variants. The analysis indicates that 93.1% of the UniProtKB HIV-1 p24 entries reveal the sequence MYSP[T/V]SILDI[K/R] (SEQ ID NO: 339) at this position, so we should have reasonable coverage of the variation even if a monoclonal antibody raised against MYSPTSILDIR (SEQ ID NO: 7) fails to sensitively bind the MYSPSSILDI[K/R] (SEQ ID NO: 340) and MYSPSSILDI[K/R] (SEQ ID NO: 340) variants of PEPTIDE3, which account for another 4% of the variation of this peptide. However, both these substitutions could be considered conservative to with respect to the amino acid variants already recognized by our current polyclonal antibodies, as each differ be the gain or loss of a single hydrogenated carbon functional group (T to S, terminal loss; V to I, internal gain).

TABLE 7
Estimate of the frequency of specific PEPTIDE1 variants in
UniProKB data HIV-1 gag sequence. Table discloses SEQ ID NOS
341, 5, 342-347, and 337, respectively, in order of appearance.

| Short region | Variant1 | Variant2 | Total |
|---|---|---|---|
| *TINEEAAEWDR | ETINEEAAEWDR | DTINEEAF[[f]]AEWDR | [E/D]TINEEAAEWDR |
| 55,646 | 31,595 | 23,965 | 55,560 (91.3%) |
| *TINEEAADWDR | ETINEEAADWDR | DTINEEAADWDR | [E/D]TINEEAADWDR |
| 3,346 | 2,238 | 1,099 | 3,337 (5.5%) |
| | | | 58,897 (96.8% of 60,829 |
| | | | GSDIAGTTS entries) |

### Example 5—Multiplex Serum Detection of HIV-1 and Mycobacterium tuberculosis Infections by a Rapid Affinity-Enriched Mass Spectrometry Assay

Co-infection with HIV and Mycobacterium tuberculosis (Mtb) significantly contributes to the global morbidity and mortality rates of these diseases. Improving early diagnosis rates and monitoring of the treatment responses of individuals with both infections is essential to improve patient outcomes. PCR-based tests exist for each pathogen but have limitations that hinder their utility for joint screening approaches, particularly since the only United States Food and Drug Administration-approved assay is only approved for sputum specimens. This significantly limits its diagnostic utility in patients co-infected with HIV and Mtb (HIV/Mtb cases), who frequently produce sputum specimens with low Mtb abundance. Herein, we describe the development of a diagnostic assay that directly quantifies the level of single peptides of HIV-1 p24 and Mtb culture filtrate protein 10 (CFP-10) to evaluate the systemic burden of each pathogen and its response to treatment. Results from a case control study found that real-time multiplex quantification p24 and CFP-10 target peptides in patient blood specimens was informative for early HIV and Mtb diagnosis and in assessing patient responses to anti-Mtb and anti-retroviral therapy, indicating this approach can provide clinical information that could facilitate the linkage of Mtb and HIV prevention, diagnosis, treatment and care services to improve patient outcomes.

### Introduction

The HIV pandemic presents a significant challenge for global TB control efforts, since people living with HIV (PLHIV) have a 20- to 30-fold increased risk of developing TB, which is the leading cause of morbidity and mortality in this group [1]. PLHIV accounted for 1.2 of the estimated 10.4 million new TB cases in 2016, and 0.4 of the 1.7 million TB deaths [1]. Improving the rate of TB testing in PLHIV and HIV testing in TB patients is a critical step for reducing the prevalence of HIV/TB co-infection by increasing the rate of early treatment to improve patient outcomes. The 2013 Global Tuberculosis Report of the World Health Organization (WHO) recommended regular screening for TB during primary care visits related to HIV screening and treatment [2]. This is required to address the finding that nearly 60% of TB cases in PLHIV are not diagnosed or treated, leading to excess morbidity and mortality [1]. Similarly, the target goal of the Stop TB Partnership's Global Plan to Stop TB was that by 2015 all patients with TB should be tested for HIV [3]. However, in 2016 only 57% of the new and relapsed global TB cases had a documented HIV test. [1].

The primary reasons for this diagnostic gap may include socioeconomic factors associated with HIV/TB, including poverty, poor knowledge of and attitudes about HIV/TB infection, and adverse patient behaviors [4]. However, patients with HIV-associated TB (HIV/TB) often have their TB and HIV managed in separate programs provide treatment for each disease but do not coordinate these therapy regimes. This lack of coordination may be a significant factor in missed or delayed HIV and/or TB diagnosis and treatment, contributing to preventable morbidity and mortality. WHO policy emphasizes the need to establish mechanisms for delivering integrated TB and HIV services, for example at the same time and location [5]. Intensifying TB case-finding in PLHIV and providing HIV testing and counseling to patients with presumptive and diagnosed TB cases are believed to be key requirements to mitigate the burden of TB/HIV in populations at risk of developing, or affected by, both diseases [6].

To further complicate this situation, TB diagnosis in PLHIV presents a major challenge, since PLHIV with TB frequently have “subclinical” TB cases that are often not recognized as TB, delaying their TB diagnosis and treatment. Sputum-based TB diagnostics, including acid-fast bacilli (AFB) smear, Mtb culture, and the GeneXpertR MTB/RIF, exhibit reduced sensitivity with paucibacillary (low bacillary concentration) samples that are frequently associated with HIV/TB cases [7, 8]. New approaches that allow sensitive and specific diagnosis of HIV/TB infections would therefore address an urgent unmet need to improve the integrated management of HIV-1 and TB disease.

Detection and quantification of a pathogen-specific protein in serum or plasma provides direct evidence of infection and can also reflect pathogen burden and thus can be used to both diagnose an infection and monitor its response to treatment. However, conventional immunoassays for such biomarkers are faced with confounding issues. For example, interactions with circulating factors, including antibodies, may significantly attenuate immunoassay sensitivity, while off-target recognition of homologs from related pathogens, such as nontuberculous mycobacteria (NTM), can reduce assay specificity.

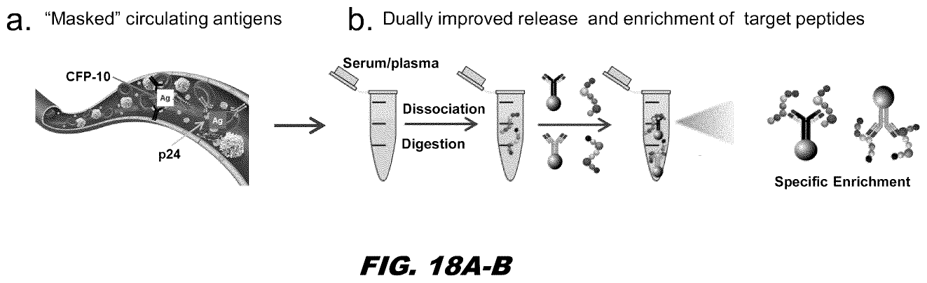

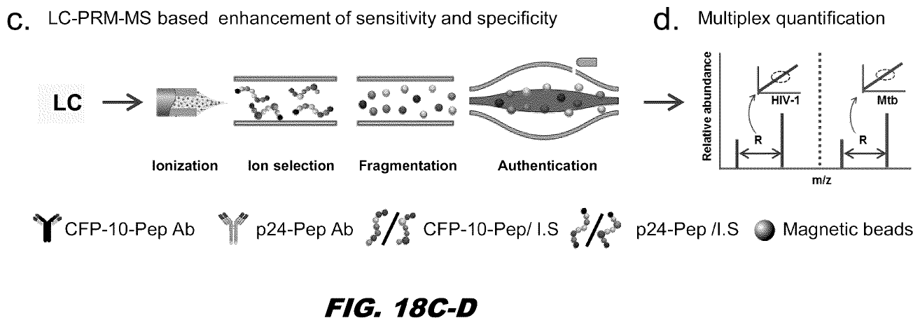

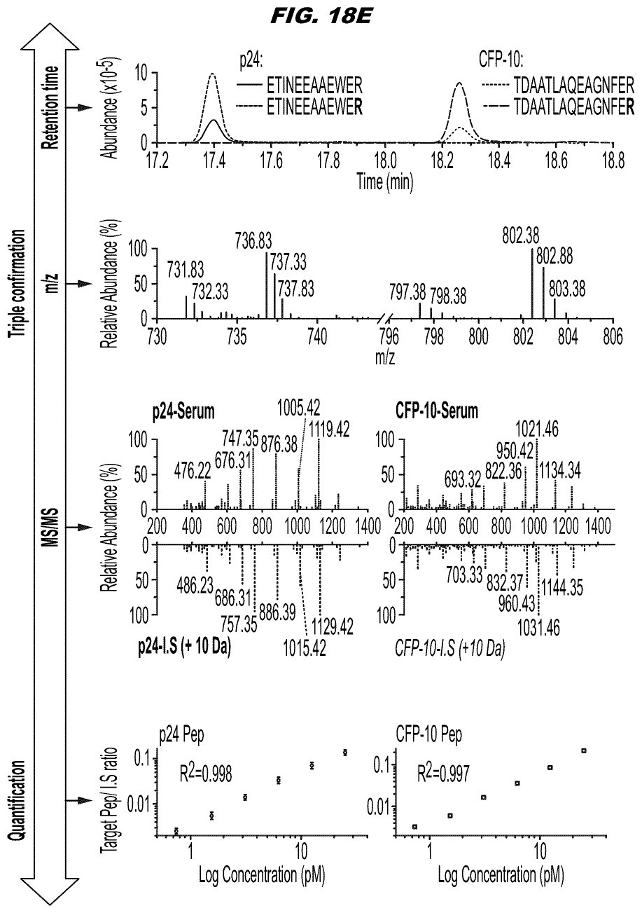

Assays that measure proteotypic biomarker peptides can avoid these issues since the digestion step required to produce these peptides disrupts protein-protein interactions, while the released biomarker peptides can often differentiate between highly similar proteins and their isoforms. We have previously reported that detection of Mtb-derived peptides in patient plasma or serum by mass spectrometry (MS) can diagnose active pulmonary and extrapulmonary TB cases with high sensitivity and specificity in the presence or absence of HIV co-infection [9, 10]. In the current study, we developed a liquid chromatography (LC) MS method that detects and quantifies peptides derived from the Mtb virulence factor culture filtrate protein 10 (CFP-10), and the HIV-1 capsid protein p24 in a single MS spectrum for rapid diagnosis and real-time treatment monitoring of HIV-1/TB co-infections. In this method, target CFP-10 and p24 peptides from trypsin digested plasma or serum samples are analyzed by immunoprecipitation followed by scheduled parallel reaction monitoring (iSPRM) in an LC-iSPRM-MS approach that allows rapid quantification of HIV-1-and Mtb-specific peptides. This assay approach contains multiple features that enhance the sensitivity and specificity of biomarker detection (FIG. 18). Simultaneous and quantitative monitoring of Mtb and HIV-1 infections offered by this assay could open new possibilities for the diagnosis and management of patients with immune reconstitution inflammatory syndrome (IRIS) and facilitate the discrimination of IRIS from TB treatment failure.

We chose CFP-10 as a biomarker of active TB, since it is actively secreted by virulent Mtb strains, detectable soon after infection, attenuates Mtb clearance and can be readily detected in plasma and serum for active TB diagnosis in adults [11]. We selected p24 as a biomarker of HIV-1 infection, since its detection has been shown to be of value in: (i) diagnosing early HIV-1 infections; (ii) screening blood to identify samples from HIV-infected donors; (iii) diagnosing HIV infections in newborns; and (iv) monitoring anti-retroviral therapy (ART) efficacy, provided that the p24 levels is measured with sufficient sensitivity and accuracy [12].

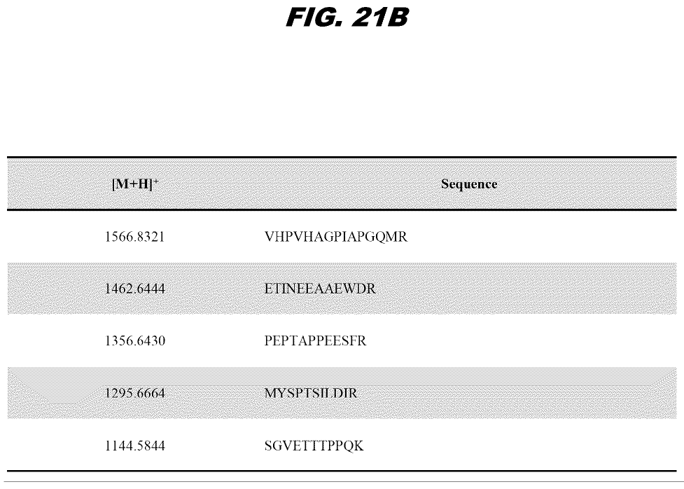

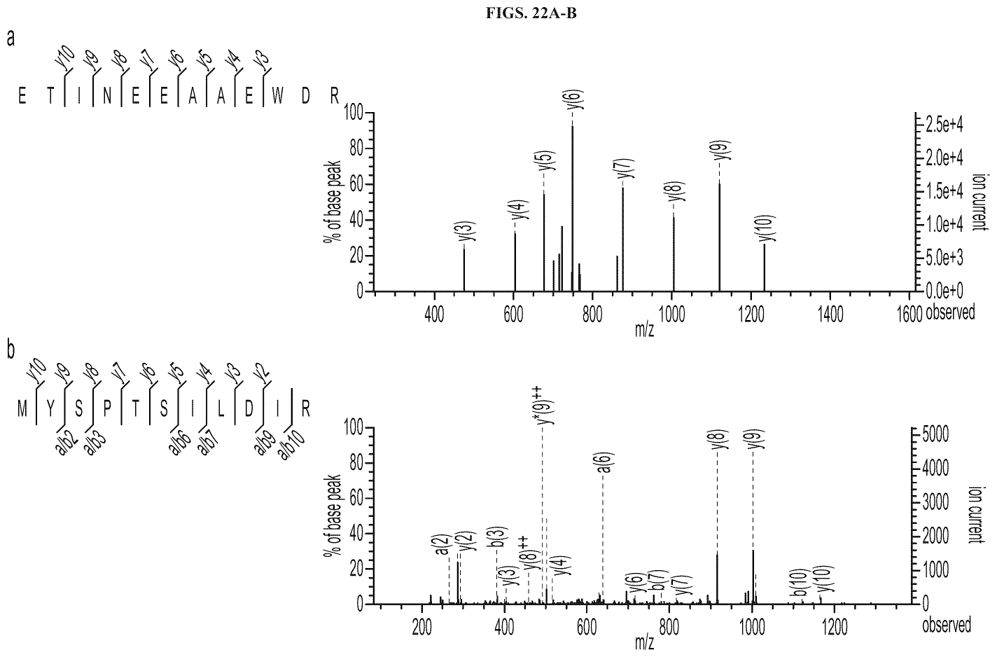

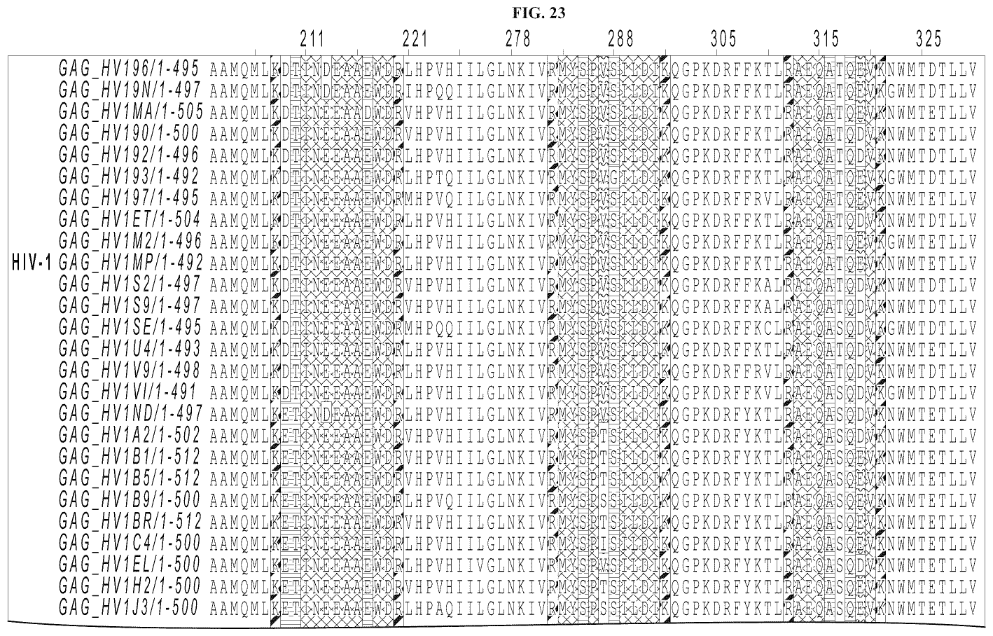

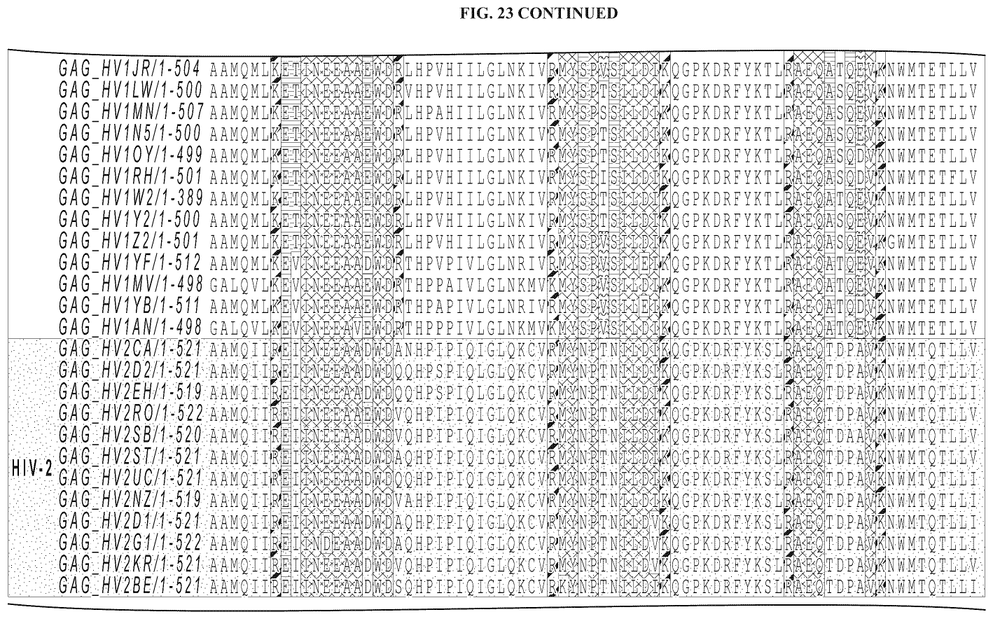

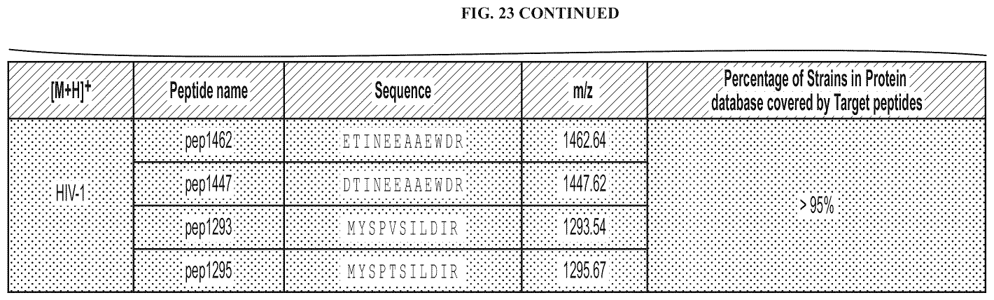

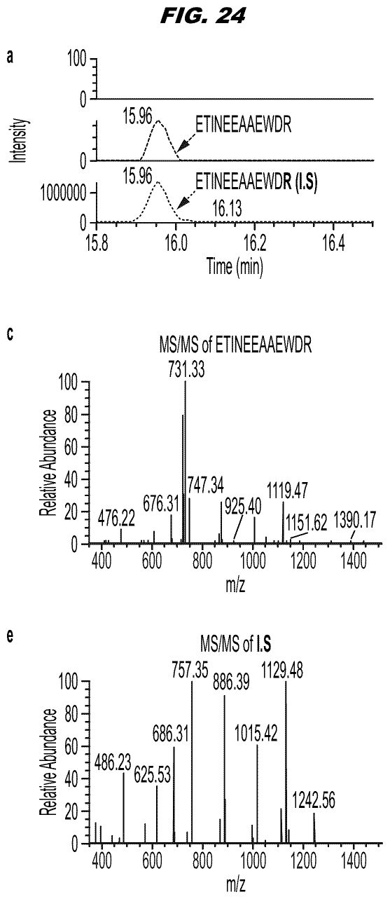

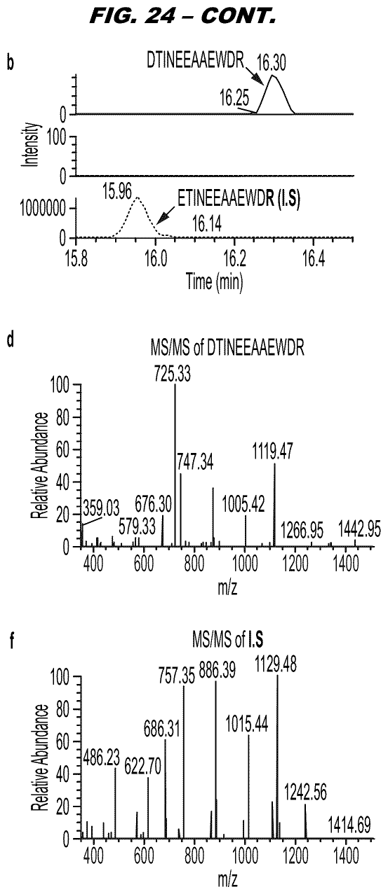

We have previously identified and validated a peptide that can identify and quantitate CFP-10 present in patient serum and plasma samples for TB diagnosis [9, 10]. Proteotypic p24 peptides intended for HIV-1 diagnosis were identified by subjecting tryptic digests of recombinant HIV-1 p24 to matrix-assisted laser desorption/ionization time-of-flight MS and LC electrospray ionization tandem MS analysis. Peptides ETINEEAAEWER (SEQ ID NO: 13) (m/z 1462.83) and MYSPTSILDIR (SEQ ID NO: 7) (m/z 1295.43) detected in this analysis demonstrated signal-to-noise ratios >125 and exhibited HIV-1 specificity when aligned with p24 protein sequence of HIV-1 and HIV-2 (FIGS. 21-23). These peptides, and their variants DTINEEAAEWER (SEQ ID NO: 348) and MYSPVSILDIR (SEQ ID NO: 9), in aggregate matched the p24 sequence of >95% of the analyzed HIV-1 strains, but not HIV-2 p24 sequence. Antibodies raised against ETINEEAAEWER (SEQ ID NO: 13) and MYSPTSILDIR (SEQ ID NO: 7) also efficiently bound DTINEEAAEWER (SEQ ID NO: 348) and MYSPVSILDIR (SEQ ID NO: 9) (FIG. 24, and not shown).

To evaluate the ability of LC-iSPRM-MS to quantify p24 and CFP-10 in human serum, pooled serum from healthy subjects was spiked with recombinant p24 and CFP-10 protein, trypsin digested, and spiked with stable-isotope-labeled internal standard (IS) peptides matching the sequence of the target p24 and CFP-10 peptides, after which both the target and IS peptides were immunoenriched and quantified by LC-iSPRM-MS at high sensitivity. All peptides were detected with high specificity due to the multiple levels of LC-iSPRM-MS data confirmation: retention time, mass-over-charge (m/z) ratio and the MS/MS spectra of the target and internal standard peptides (FIG. 18b). Standard curves generated by plotting the ratio of the MS peak intensity for a target peptide and its corresponding IS in serum samples spiked with known amounts of p24 and CFP-10 exhibited strong linear correlation (R²>0.99) with the amount of input protein (FIG. 18b) and reproducibility (14-22% within-run and 16-23% between-run coefficients of variation). In this analysis, the p24 ETINEEAAEWER (SEQ ID NO: 13) and CFP-10 TDAATLAQEAGNFER (SEQ ID NO: 1) peptides demonstrated limits of detection (LOD) and quantitation (LOQ) of 0.1 pM and 0.5 pM, respectively, while the p24 MYSPTSILDIR (SEQ ID NO: 7) peptide exhibited a 1.0 pM LOD and 2.5 PM LOQ.

We next evaluated the ability of this assay to diagnose HIV-1 infections in a cohort of adults (31 HIV+ and 25 HIV− individuals) enrolled in the Houston Tuberculosis Initiative (HTI), a large, population-based TB surveillance study. This assay was also applied to diagnose HIV in a group of infants (16 HIV⁺ and 10 HIV⁻) enrolled in the International Maternal Pediatric Adolescent AIDS Clinical Trials (IMPAACT) P1041 trial, a TB prevention trial conducted among infants enrolled from 2004 to 2008 during South Africa's roll-out of combination antiretroviral therapy (ART) (Table 8).

TABLE 8
Demographics and clinical characteristics of the
study participants for the diagnosis of HIV-1

| | Controls | HIV-1 cases | p Value |
|---|---|---|---|
| Adult (HTI Cohort) | n = 25 | n = 31 | |
| Sex, male (%)^{a} | 18 (72) | 23 (74.2) | 0.717^{c} |
| Age, years (IQR)^{a} | 46 (40-57) | 37 (36-44) | 0.0025^{c} |
| Log¹⁰ CD4 T cells/μL | — (—) | 1.8 (1.6-2.2) | — |
| (IQR)^{a, b} | | | |
| Log¹⁰ HIV copies/mL(IQR)^{a} | — (—) | 5.5 (4.6-5.7) | — |
| Infants (p1041 Cohort) | n = 10 | n = 16 | |
| Age, months | 3-4 | 3-4 | — |
| Log¹⁰CD4 T cells/μL | — (—) | 3.3 (3-3.4) | — |
| (IQR)^{a, b} | | | |
| Log¹⁰ HIV copies/mL (IQR)^{a} | — (—) | 5.7 (4.6-6) | — |

Data, no. (% or IQR)
IQR interquartile range; n/a not available.
^{a}Percentage or interquartile range of corresponding column population.
^{b}CD4 cell counts were only available for HIV-1 positive subjects.
^{c}p value of Student t test, Mann-Whitney U test, or chi-square test for difference between controls and HIV-1 cases.

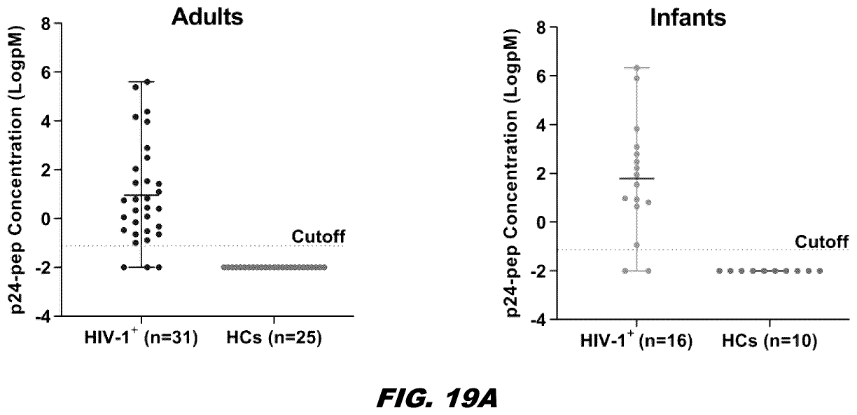

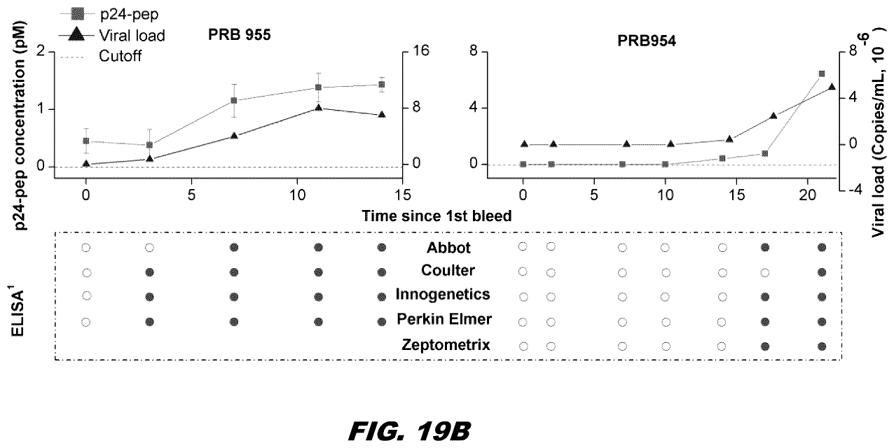

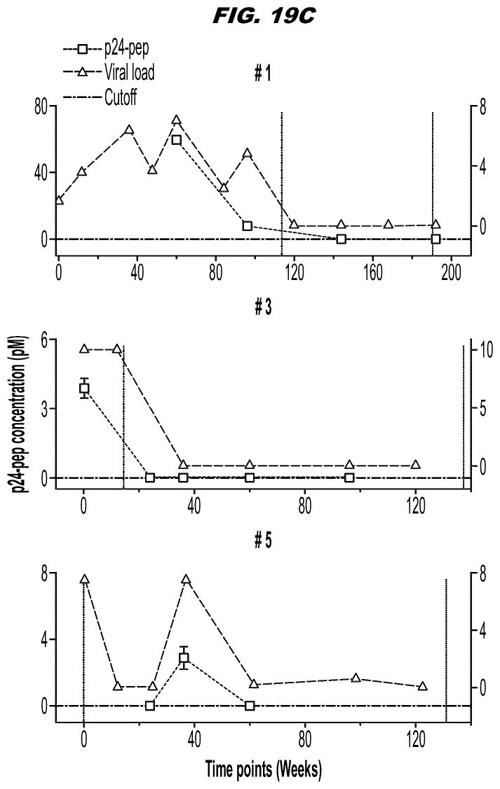

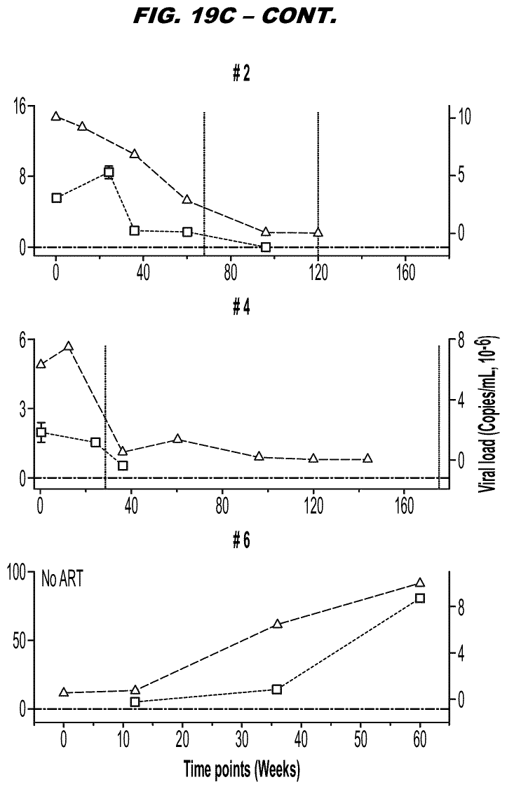

The specificity of this assay for diagnosis of HIV-1 infections was assessed by analysis of 12 confirmed HIV-2 antibody-positive plasma samples purchased from SeraCare Life Sciences Inc. (Milford, MA, USA). LC-iSPRM-MS revealed similar diagnostic sensitivity for HIV-1 cases in HTI adults (90.3%; 95% CI: 74.25-97.96%) and P1041 infants (87.5%; 61.65-98.45%), and 100% specificity in both populations (FIG. 19a and Table 10), which would be useful for the diagnosis of infant HIV-1 cases that are often difficult to diagnose using antibody-based assays due to the presence of maternal HIV antibodies during the first year of life [13, 14]. No false positives were found in the tested HIV-2 samples, confirming the specificity of this assay for the diagnosis of HIV-1 infections.

TABLE 10
Sensitivity and specificity of LC-iSPRM-MS for HIV-1 detection

| | Positive results/ | Sensitivity, | Specificity, |
|---|---|---|---|
| Cohort | total no. | % (95% CI) | % (95% CI) |
| Adult | | | |
| (HTI Cohort) | | | |
| HIV-1+ | 28/31 | 90.3 (74.25-97.96) | |
| Non-HIV-1 | 0/25 | | 100 (86.28-100) |
| Infants | | | |
| (p1041 Cohort) | | | |
| HIV-1+ | 14/16 | 87.5 (61.65-98.45) | |
| Non-HIV-1 | 0/10 | | 100 (69.15-100) |

Accurate diagnosis of acute HIV-1 infection via a p24 assay depends on its ability to sensitively detect p24 sequence variants expressed by different HIV-1 strains. Several factors (e.g., testing method, individual responses and viral characteristics) influence the interval between virus exposure and the earliest reliable detection of HIV RNA, HIV antigens, or anti-HIV antibodies in serum samples. To evaluate LC-iSPRM-MS performance for early HIV diagnosis, we analyzed serum samples obtained from two patients before and after they tested positive for HIV-1 and found that in each case LC-iSPRM-MS detected p24 expression at least one time point earlier than p24 immunoassay (FIG. 19b). LC-iSPRM-MS detected 8 of the 9 HIV⁺ specimens (88.9% sensitivity) versus the 5 to 6 samples detected by p24 ELISA indicating a range of 55.6% to 66.7% sensitivity. LC-iSPRM-MS detected serum p24 expression one sample earlier than the Innogenetics and Perkin Elmer Alliance ELISAs, and one and two samples earlier than the Coulter or Abbot ELISAs, respectively. Notably, the PRB954 samples analyzed in this study spanned the early seroconversion interval, as viral RNA was not detected in first three samples of this patient, and that LC-iSPRM-MS detected p24 expression in 3 of the 4 serum samples with detectable HIV RNA.

Regular monitoring of patients on antiretroviral therapy is critical to ensure ongoing viral suppression and, in the case of treatment failure, to detect the emergence of drug-resistant strains as early as possible. Viral load assays are universally recommended for this purpose, and while serum p24 quantification can have applications for HIV prognosis and treatment monitoring [8], it is not yet clear that it can provide useful data for monitoring HIV treatment responses. We therefore analyzed serial blood samples from HIV-1⁺ infants in the P1041 cohort before and after ART therapy and during long term follow-up and found that there was good correspondence between serum p24 level and viral load, and that these factors tended to decrease and increase in parallel in ART-treated and untreated subjects, respectively (FIG. 19c). LC-iSPRM-MS also identified ART failure in one treated patient (FIG. 19c #5), who demonstrated a p24 increase with a rise in HIV viral load during ART, and a corresponding drop in both p24 level and viral load following a HIV therapy modification. Studies with larger longitudinal cohorts are underway to assess how early changes in antigen level correspond to changes in patient symptoms and treatment outcomes.

WHO recommends integrating clinical services for patients co-infected with TB and HIV as this approach appears to be associated with lower mortality during anti-TB treatment, even in settings where suboptimal fractions of patients initiate ART and complete anti-TB treatment

. To provide proof-of-principle evidence for the clinical utility of LC-iSPRM-MS, we analyzed serum from patients with HIV-1, TB, or combined HIV and TB infections, using positive p24 or CFP-10 signal as the diagnostic criteria for an HIV or TB infection. This case-control study (Table 9) analyzed serum from 1 patient infected with HIV-1 alone, 4 infected with Mtb alone, 7 co-infected with HIV-1 and Mtb, and 8 control subjects who were not infected with HIV-1 or Mtb.

TABLE 9
Characteristics of the study population stratified by TB and HIV status

| | Total | HIV-1 negative | | HIV-1 positive | |
|---|---|---|---|---|---|
| | patients | TB negative | TB positive | TB negative | TB positive |
| | (n = 20) | (n = 8) | (n = 4) | (n = 1) | (n = 7) |
| Sex, male (%)^{a} | 20 (80) | 8 (62.5) | 4 (75) | 1 (100) | 7 (100) |
| Age, years (IQR)^{a} | 52 (41-65) | 59 (37-66) | 59 (41-79) | 46 (—) | 44 (40-54) |
| Culture-positive: no. (%)^{a} | 12 (60) | — (—) | 5 (100) | — (—) | 7 (100) |
| AFB smear positive: no. (%)^{a} | 5 (25) | — (—) | 1 (20) | — (—) | 4 (80) |

Data, no. (% or IQR)
IQR interquartile range,
AFB acid-fast bacilli
^{a}Percentage or interquartile range of corresponding column population.

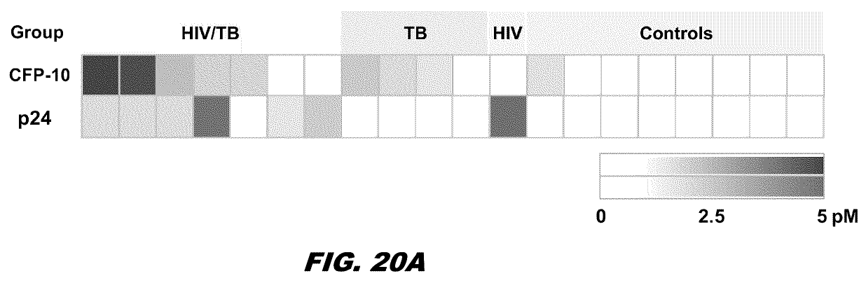

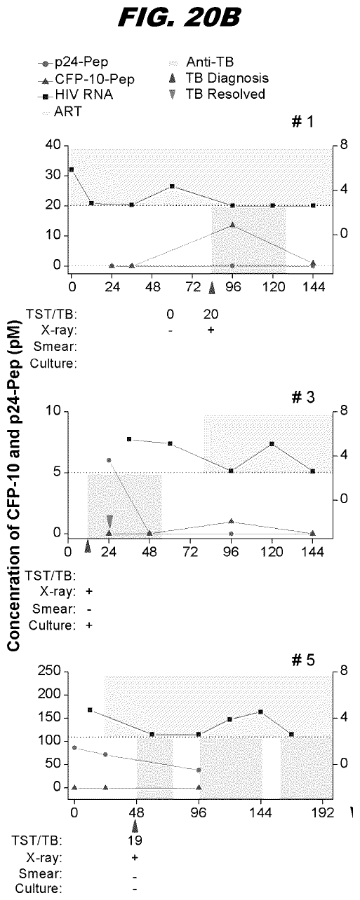

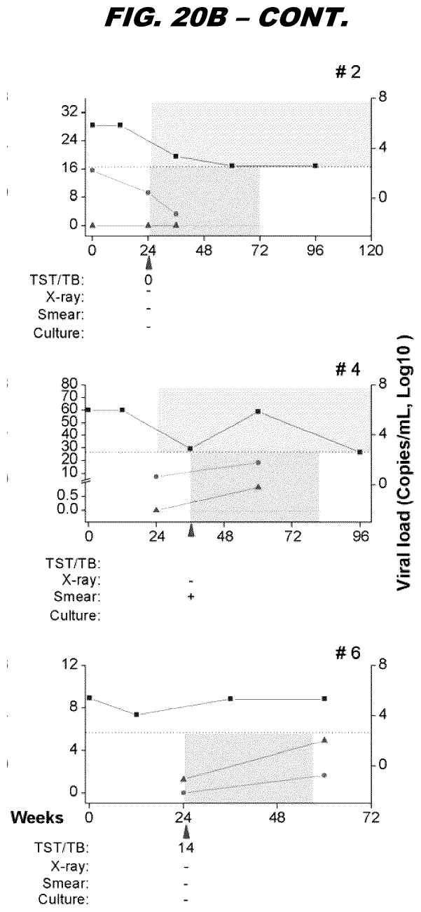

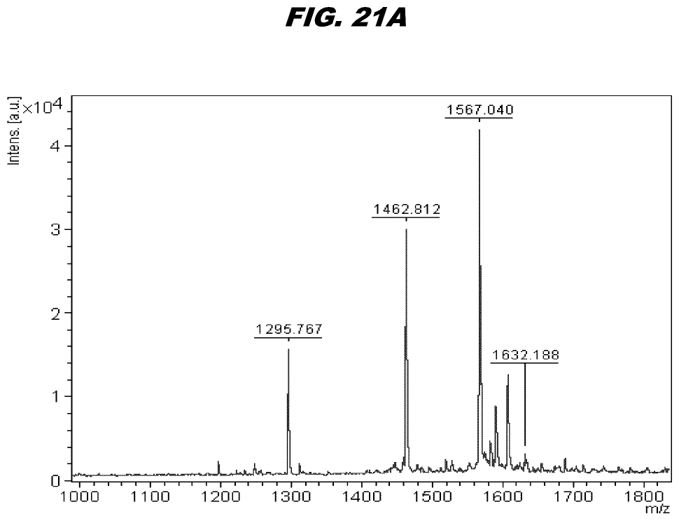

Blinded LC-iSPRM-MS assays detected CFP-10 and p24 target peptides in the serum samples of 9 of the 11 TB cases (81.8%) and 7 of the 8 HIV-1 cases (87.5%), respectively (FIG. 20a and Table 11).

TABLE 11
Sensitivity and specificity of LC-
iSPRM-MS for active TB and HIV-1

| | Positive results/ | Sensitivity, | Specificity, |
|---|---|---|---|
| Group | total no. | % (95% CI) | % (95% CI) |
| TB detection | | | |
| All TB | 9/11 | 81.82 (48.22-97.72) | |
| Culture⁺, | 4/5 | 80.0 (28.36-99.49) | |
| Smear⁺ | | | |
| Culture⁺, | 5/6 | 85.71 (42.13-99.64) | |
| Smear | | | |
| All Non-TB^{a} | 1/9 | | 88.89 (51.75-99.72) |
| HIV-1 | | | |
| detection | | | |
| HIV-1 | 7/8 | 87.5 (47.35-99.68) | |
| Non-HIV | 0/12 | | 100 (73.54-100) |

^{a}Non-TB controls are subjects from patients who were judged to not have active TB but at high risk for TB development.

No p24 signals were detected in any of the HIV subjects (100% specificity), but an apparent false-positive CFP-10 signal was detected in 1 of the 9 at-risk subjects who was judged not to have active TB (88.9% specificity). This degree of diagnostic sensitivity for TB outperformed reported sensitivity values for AFB smear (31%), mycobacteria growth indicator tube (MGIT) culture (69%), and GeneXpert® MTB/RIF (66%) in similar HIV/TB populations [15, 16] and exceeded the WHO-recommended optimal sensitivity (66%) for new high-priority non-

sputum diagnostic tests [17]. WHO guidance recommends the use of GeneXpert® MTB/RIF as an initial diagnostic in HIV⁺ patients with suspected TB, but the sensitivity of this test decreases in AFB smear-negative and Mtb culture-negative TB cases that are prevalent in HIV⁺ populations [18]. LC-iSPRM-MS sensitivity in HIV+ smear-negative/culture-positive TB cases (85.7%; 70.5-95.3) also exceeded GeneXpert® sensitivities (47.3%; 29.2-67.0 to 61.1%; 35.7-82.7) reported in other HIV+ adult cohorts [19]. Caution must be exercised in reaching any final conclusions about the performance of LC-iSPRM-MS in this population, however, due to the small sample size of this study.

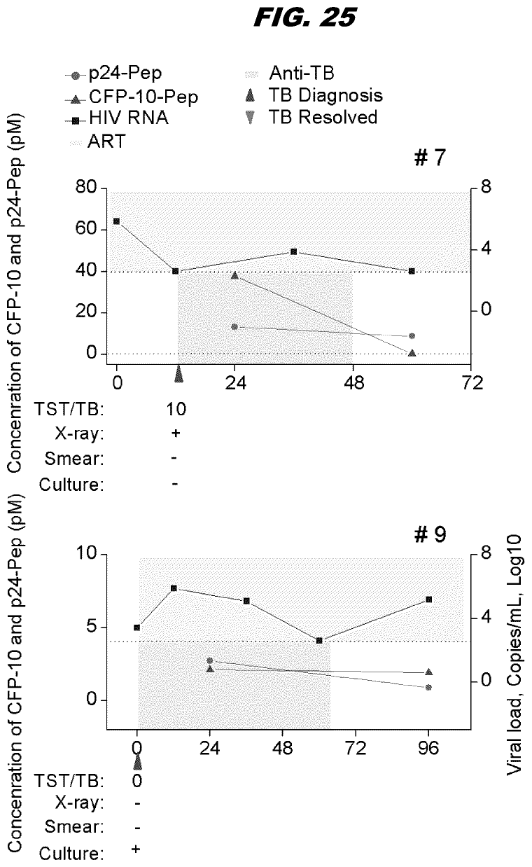

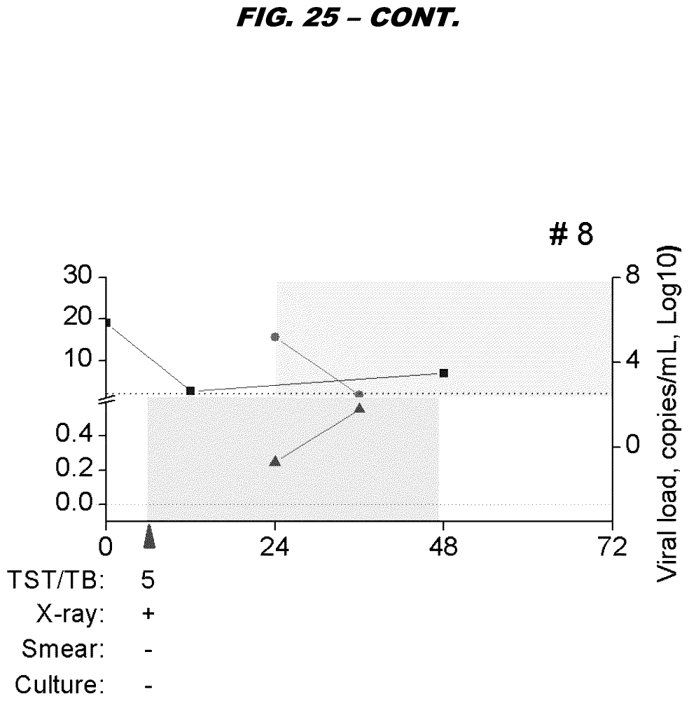

The LC-iSPRM-MS assay was also designed to quantify HIV-1 and TB antigens in serum or plasma to monitor responses to anti-TB therapy or ART. This ability is particularly useful for TB therapy, given that most assays used to monitor anti-TB treatment responses provide semi-quantitative results (AFB smear and GeneXpert®) or have significant latency (Mtb culture). Real-time monitoring of HIV-1 and Mtb antigens in serum during anti-TB therapy and/or ART may reflect therapeutic efficacy, which could distinguish treatment failure due to drug-resistant infections from drug toxicity or IRIS. To evaluate LC-iSPRM-MS performance for real-time monitoring of CFP-10 and p24 antigen levels, we analyzed serial blood samples from HIV-1 positive infants in p1041 cohort, who were receiving ART treatment and anti-TB therapy if diagnosed as TB. The results showed that LC-iSPRM-MS can successfully monitor the CFP-10 and p24 levels in real time and provide useful information for TB and HIV diagnosis, as well as for the assessment of anti-TB or ART treatment response (FIG. 20b and FIG. 25). For instance, the turning of CFP-10 signals from positive to negative in patient after completion of anti-TB treatment may reflect a successful anti-TB response (FIG. 20b #1). Similarly, after initiation of ART, the p24 levels in patient #2 showed continued decreases along with a corresponding drop in viral load, which also may represent a positive response of ART (FIG. 20b #2). By contrast, high CFP-10 or p24 levels after completion of anti-TB therapy could represent treatment failure of TB or HIV, respectively. For example, the CFP-10 signals were still very high even after 9-14 months of anti-TB therapy in patient #6 and #9, which could represent two nonresponsive cases. Actually, patient #6 was found to have very high HIV viral load (>1000, 000 copies) but no receiving ART treatment (FIG. 20b #6), while HIV virus rebounded to be very high (750,000 copies) in patient #9 even though receiving ART (FIG. 25 #9). On the other hand, the positive CFP-10 signals were detected before or shortly after TB diagnosis in several cases (FIG. 20b #1, 4, 6), which indicate that this approach may be also useful for early detection of TB infection.

Intriguingly, when correlated the serum CFP-10 signals with p24 levels or HIV viral loads, the LC-iSPRM-MS can identify TB-IRIS cases. For instance, the CFP-10 levels in patients #3 and #4 were found to be increased shortly after initiation of ART treatment even though whose HIV virus seems to be under controlled by exhibiting undetectable p24 antigen or HIV viral load (FIG. 20b #3, 4). Nonetheless, this proof-of-principle study was not designed to measure rates of decline or to allow comparison with other methods. Future prospective longitudinal studies with frequent sampling are needed to determine how HIV-1 p24 and Mtb CFP-10 serum/plasma changes correspond to patient phenotypes and treatment outcomes. These studies should, in turn, open up new possibilities for the diagnosis and management of patients with IRIS, and facilitate the discrimination of IRIS from TB treatment failure.

Real-time and multiplex LC-iSPRM-MS quantification of p24 and CFP-10 target peptides from patient blood samples was informative for early HIV and TB diagnosis and in assessing patient responses to anti-TB and anti-retroviral therapy in this study. These findings suggest this approach can provide clinical information that could facilitate the linkage of TB and HIV prevention, diagnosis, treatment and care services to improve patient outcomes. Nonetheless, further reductions in operator time and assay cost, along with improvements in instrument portability, are needed to meet WHO guidelines for an optimal noninvasive TB assay.

### Materials and Methods

### Commercial Plasma and Serum Samples

Confirmed HIV-2 antibody-positive plasma samples (9227022, 9250494, 9227024, 10234820, 10266767, 10276442, 10266768, 10279915, 9226992, 10296579, 10231111 and 10266760) were purchased from SeraCare Life Sciences Inc. (Milford, MA, USA) as well as the HIV-1 seroconversion panels PRB955 and PRB954, which included samples drawn before HIV-1diagnosis and throughout the immune response to the ongoing HIV-1 infection.

### Clinical Samples

The HTI cohort that served as a source of case and control samples for adults in this study was a large, population-based TB surveillance study that performed active surveillance of all confirmed TB cases in Houston/Harris County, Texas between 1995 and 2004. Because of its mandate to collect all active TB cases, the HTI archived samples from a variety of TB disease manifestations, including HIV-negative and-positive pulmonary and extrapulmonary TB cases with both positive and negative culture results. Serum samples were obtained from HTI subjects who were notified of the risks of study participation and provided written informed consent. Demographic, microbiology, and diagnostic data are summarized in (Table 8).

The infant samples used in this study were part of serum samples collected form IMPAACT P1041 cohort, which was a multi-center, Phase II-III, randomized, double-blind, placebo-controlled trial evaluating primary isoniazid (INH) prophylaxis in healthy, bacille Calmette-Guérin (BCG) vaccinated, 3- to 4-month-old infants. P1041 enrolled 804 HIV-exposed, uninfected and 547 HIV-infected infants who were followed for up to 4 years. Scheduled visits were quarterly for the first two years for all infants but were reduced to 6-monthly in years 3-4 for those HIV-exposed, uninfected.

### Pretreatment of Serum Plasma

In clinical HIV samples, p24 antigen is present within intact whole HIV viruses and free in solution after viral lysis. However, the availability of analyte is limited by low viral titers in addition to the formation of immune complexes after seroconversion. So to disrupt immune complexes due to maternal antibodies to further improve the trypsin digestion, we employed a heat disruption pretreatment step that denatured the immune-complexing antibodies as well as virus particles by heating the samples to 100° C. for 5 minutes. After allowing the samples to cool to room temperature (25° C.), p24 antigen is available for detection. We empirically tested a range of detergents diluted in phosphate-buffered saline that facilitated heating of plasma to denaturing conditions while precluding the formation of gelled samples. Our chosen buffer, consisting of SDS and NP-40, was robust in allowing a heat spike when a plasma sample is diluted 1:3 in the heat shock buffer (0.4% TritonX-100, 0.2% SDS in PBS, pH=7.4).

### LC-iSPRM-MS Analysis

100 μg of the custom polyclonal antibody (GL Biochem) raised against the HIV-1 and Mtb CFP-10 target peptides were coupled to 10 mg of the Dynabeads® M-270 Epoxy (Thermo Fisher Scientific) as described in the manufacturer's instructions, respectively. The final bead concentration is 10 mg/mL antibody coupled beads and stored at 2° C. to 8° C. until use. Serum samples (100 μL) were diluted with 300 μL of heat shock buffer and incubated to 100° C. for 5 minutes. After the samples cooled down to room temperature (25° C.), the samples were diluted with 1100 μL of 100 mM ammonium bicarbonate, then microwave digested [15], spiked with 300 pmol/L stable-isotope-labeled internal standard peptides (m/z of 1472 or 1305 for HIV-1 and m/z of 1603.60 for Mtb; GenScript) matching the p24 and CFP-10 target peptide sequences (m/z of 1462 or 1295 for HIV-1 and m/z of 1593.75 for Mtb), respectively, and mixed with 30 μL prepared anti-1462/1295 and anti-1593 antibody-labeled beads for 1 h at room temperature. Beads were then washed with PBS/0.05% Tween-20 and incubated with 1% formic acid (pH<2.0) to elute bound peptides. Eluates were loaded on a C18 trap column, eluted onto a C18 analytical column, and fractionated with a 0.3 μL/min acetonitrile/formic acid gradient (5-40%) and analyzed using the PRM Mode on a nano-LC UltiMate 3000 high performance liquid chromatography (HPLC) system coupled with an Orbitrap Fusion™ Lumos™ Tribrid™ Mass Spectrometer (Thermo Fisher Scientific). Skyline software version 4.1.0.18169 (MacCoss Lab Software) was used to analyze serum MS and MS/MS spectra against a library produced using recombinant p24 and CFP-10 digests. Standard curves were generated by spiking healthy donor serum with 0-400 pmol/L recombinant p24 or CFP-10 and converting experimental sample MS intensity ratios to absolute molar concentrations by substitution into these calibration curves. Limits of detection (LOD) and quantification (LOQ) were obtained from the mean of each blank plus three times or ten times the standard deviation of their noise, respectively.

### Statistical Analysis

Calculation of median, interquartile range (IQR), sensitivity and specificity, data normality, analysis of variance (ANOVA) with post-test correction (Dunn's test), Mann-Whitney, and chi-square tests were performed with GraphPad Prism software (version 7.01).

### REFERENCES FOR THIS EXAMPLE

1. World Health Organization (WHO). Global tuberculosis report 2018. https://apps.who.int/iris/bitstream/handle/10665/274453/9789241565646-eng.pdf?ua=1.
2. World Health Organization. Global Tuberculosis Report. Geneva: World Health Organization; (2013).
3. “The Global Plan to Stop TB”, WHO, Geneva, 2011, 12 www.stoptb.org/global/plan/
4. Kigozi N G, Heunis J C, Engelbrecht M C, Janse van Rensburg A P, van Rensburg HCJD. Tuberculosis knowledge, attitudes and practices of patients at primary health care facilities in a South African metropolitan: research towards improved health education. BMC Public Health. 2017; 17(1): 795.
5. WHO Policy on Collaborative TB/HIV Activities. Guidelines for National Programmes and Other Stakeholders. Geneva: World Health Organization; 2012.
6. Boni J, Opravil M, Tomasik Z, et al. Simple monitoring of antiretroviral therapy with a signal-amplification-boosted HIV-1 P24 antigen assay with heat-denatured plasma. AIDS 1997; 1: F47-52.
7. Evans CA. GeneXpert—a game-changer for tuberculosis control? PLOS Med. 2011; 8(7): e1001064.
8. Pai M, Schito M. tuberculosis diagnostics in 2015: landscape, priorities, needs, and prospects. J Infect Dis. 2015; 211 suppl 2: S21-8.
9. Liu C, Zhao Z, Fan J, Lyon C J, Wu H J, Nedelkov D, Zelazny A M, Olivier K N, Cazares L H, Holland S M, et al. Quantification of circulating Mycobacterium tuberculosis antigen peptides allows rapid diagnosis of active disease and treatment monitoring. Proc Natl Acad Sci U S A. 2017; 114(15): 3969-74.
10. Fan J, Zhang H, Nguyen D T, Lyon C J, Mitchell C D, Zhao Z, Graviss E A, Hu Y. Rapid diagnosis of new and relapse tuberculosis by quantification of a circulating antigen in HIV-infected adults in the Greater Houston metropolitan area. BMC Med. 2017, 15(1): 188.
11. Cardona P-J (2012) Understanding Tuberculosis: Deciphering the Secret Life of the Bacilli (In Tech, Rijeka, Croatia).
12. Schüpbach J. Viral RNA and p24 antigen as markers of HIV disease and antiretroviral treatment success. Int Arch Allergy Immunol. 2003; 132(3): 196-209
13. Donovan M, Palumbo P. Diagnosis of HIV: challenges and strategies for HIV prevention and detection among pregnant women and their infants. Clin Perinatol. 2010; 37(4): 751-763, viii.
14. Read J S, Committee on Pediatric AIDS, American Academy of Pediatrics. Diagnosis of HIV-1 infection in children younger than 18 months in the United States. Pediatrics. 2007; 120(6): e1547-1562.
15. World Health Organization (2013) Policy Update: Xpert MTB/RIF Assay for the Diagnosis of Pulmonary and Extrapulmonary TB in Adults and Children. (WHO, Geneva).
16. Theron G, Peter J, van Zyl-Smit R, Mishra H, Streicher E, Murray S, Dawson R, Whitelaw A, Hoelscher M, Sharma S, et al. Evaluation of the Xpert MTB/RIF assay for the diagnosis of pulmonary tuberculosis in a high HIV prevalence setting. Am J Respir Crit Care Med. 2011; 184(1): 132-40.
17. Joseph S. Cavanaugh, Surbhi Modi, Susan Musau, Kimberly McCarthy, Heather Alexander, Barbara Burmen, Charles M. Heilig, Ray W. Shiraishi, Kevin Cain. Comparative yield of different diagnostic tests for tuberculosis among people living with HIV in western Kenya. PLOS One. 2016; 11(3): e0152364
18. Zeka A N, Tasbakan S, Cavusoglu C. Evaluation of the GeneXpert MTB/RIF Assay for Rapid Diagnosis of Tuberculosis and Detection of Rifampin Resistance in Pulmonary and Extrapulmonary Specimens. J Clin Microbiol. 2011; 49(12): 4138-41.
19. World Health Organization (2014) High-Priority Target Product Profiles for New Tuberculosis Diagnostics: Report of a Consensus Meeting. (WHO, Geneva).

### Example 6—Added Value/Improvements to Protocol for TB-Detection and TB-Diagnostic Kit

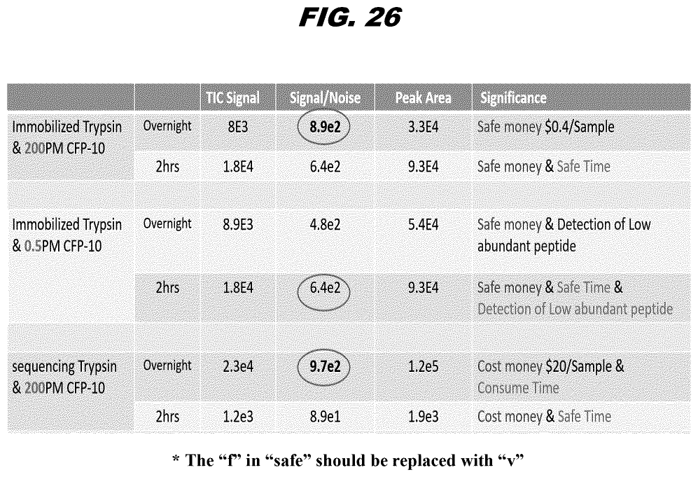

Using half of the required patient's plasma/serum volume (100 ul instead of 200 ul) which minimize the volume required to be collected from the patient and add facilitate the sample result confirmation by having enough material for repeating the assay.
Using Immobilized TPCK Trypsin instead of Sequencing grade Trypsin which add multiple values (FIG. 26) including:
a. Reduce the cost of sample from ($0.4/sample vs $20/sample for the sequencing grade)
b. Speeding up the process by allowing automation for high throughput processing

Reduce the sample digestion time from overnight to as short as two hours.

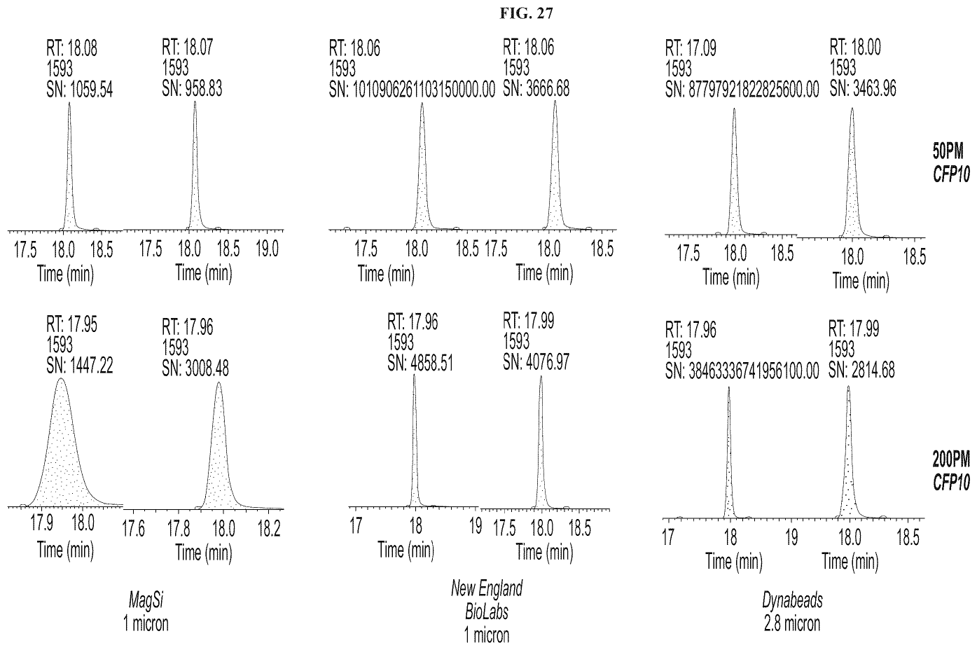

Using variety of magnetic nanoparticles from different manufacturers and with various sizes (1 micron and 2.8 micron) not only show the versatility and applicability of our optimized protocol (FIG. 27) but also provide platform for high throughput automation and Secure the supply chain and resources for one of the most value items in the diagnostic kit and in vitro protocol.

Simplifying the protocol by omitting unneeded steps and materials such as 10% TFA in the immunoprecipitation step.

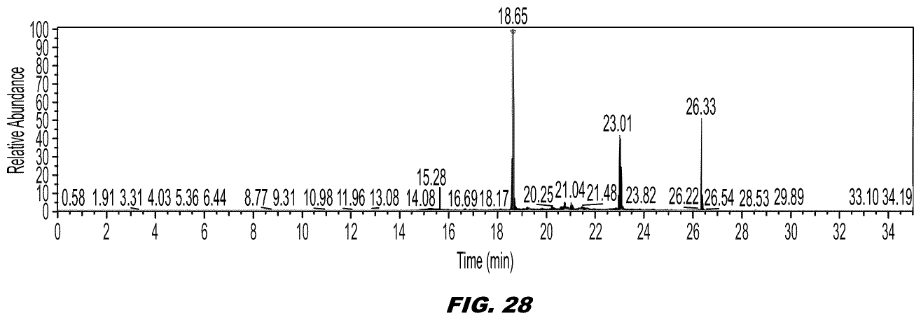

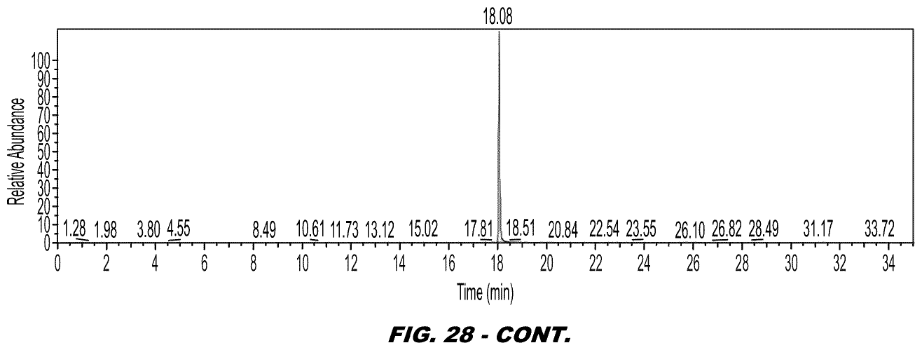

Improving the purity by removing the unspecific binding using simple was step with 75% acetonitrile/25% PBS for 30 seconds (FIG. 28).

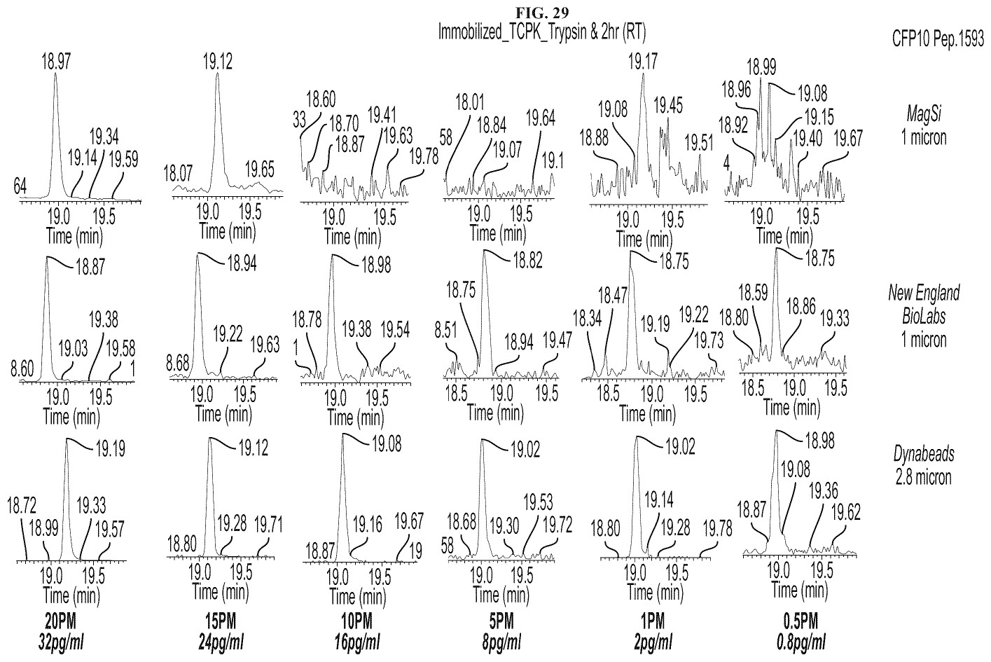

Achieving markedly low Limit of Detection (LOD) ˜0.8 pg/ml of the pathogen target peptide 1593 in a complex biological serum/plasma samples with at least two different G-Protein magnetic beads platform (FIG. 29).

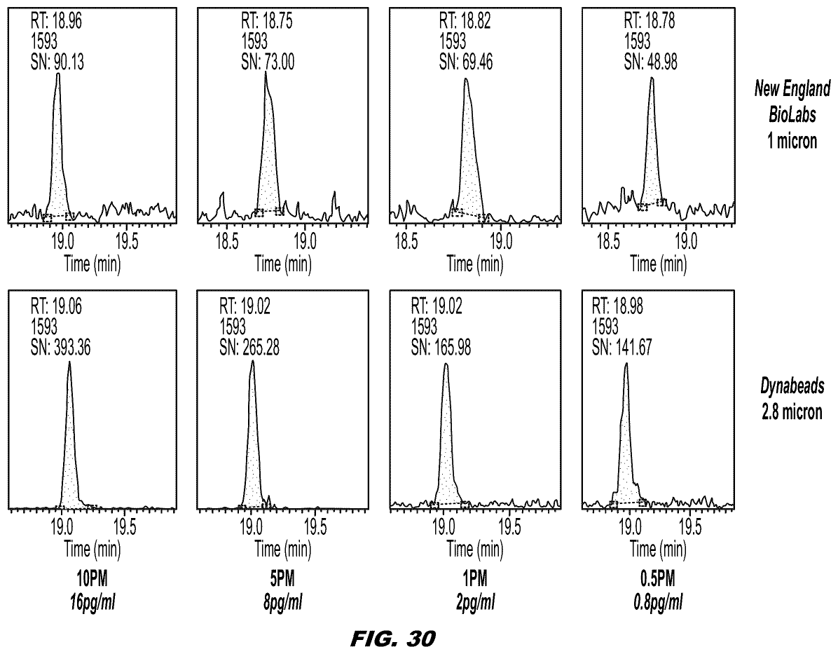

Validating our protocol using top-of-the-line ultra-sensitive Thermo TSQ Altis quadrupole mass spectrometer using nanoflow with that allow us to detect as low concentration as sub-picomolar of the target peptide 1953 (FIG. 30).

The present invention can be used on multiple mass spectrometry platforms. Non-limiting examples include Thermo TSO Altis, Waters XevoTQ-XS, and SCIEX 6500+. For example, analytical validation using ultra-sensative quadrupole mass spectrometer from two other manufacturers Waters Xevo TQ-XS and SCIEX 6500+ can be used to show the versatility and applicability of the protocols described herein.

Shortening the LCMS analysis time of each sample from 35 min to 10 min or less are undergoing using both nanoflow and microflow platform with multiple manufacturers.

The present invention has been described in terms of one or more preferred embodiments, and it should be appreciated that many equivalents, alternatives, variations, and modifications, aside from those expressly stated, are possible and within the scope of the invention.

### Example 7: The Selection Process of the Spectrum Identified by the Spectral Library of the Targeted Peptide in Skyline Database

1. Rank the ions based on the number of interferences they are predicted to have, from the least to the most one.
2. Rank the ions based on their relative abundances in MS spectra, from the highest to the lowest one.
3. Obtain the product ion scans spectra of the peptide target using its synthetic version (MS2 likely sspectra but using triple quadrupole instrument instead of orbitrap or ion trap.) Use the Skyline software to calculate the similarity score rdotp of the spectra by comparing it to the library spectrum and adjusting the ions used for calculation.
4. Select the most abundance ions, if two ions are with identical abundance, the one with less interferences will be kept.
5. Determine the final combination of transitions to achieve the highest rdotp.

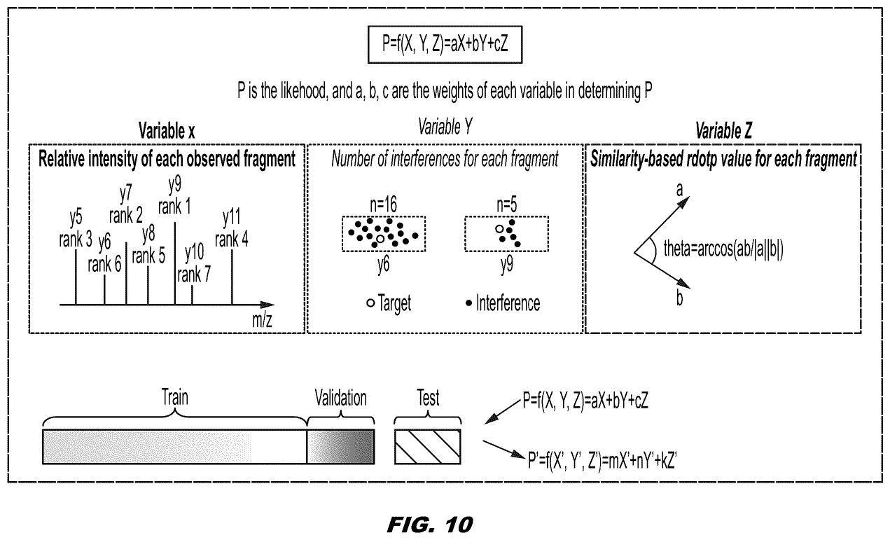

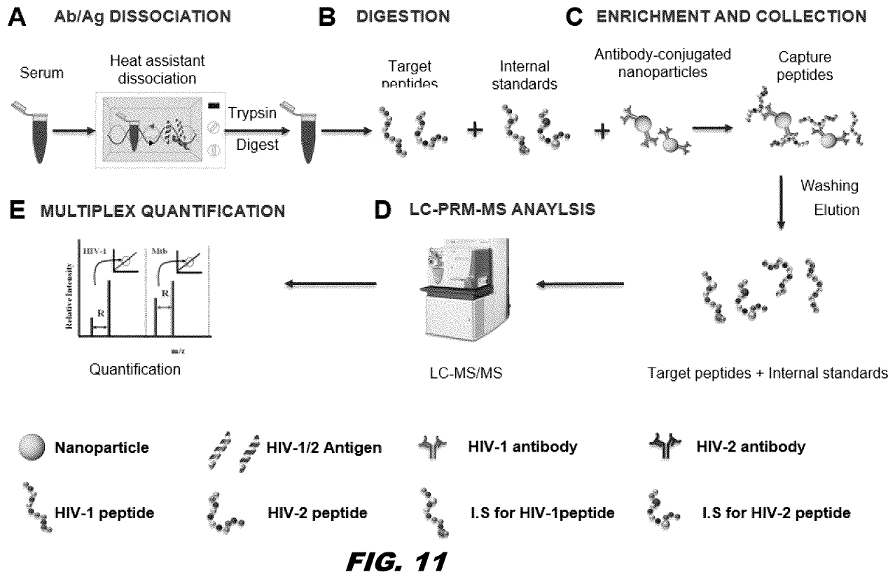

Without wishing to be bound by theory, in the FIG. 10, a machine learning based algorithm is used to assist with the selection of MRM transitions. Initially, this algorithm is used for determining the positive result, but it can also be used for MRM transition selection.

1. A kit for the detection and identification of a biomarker of disease or disorder, the kit comprising:
one or more of antibody-modified solid supports (AMSS), wherein the antibodies bind to a disease or disorder peptide or fragment thereof; and
instructions for use.

2. The kit of claim 1, wherein AMSS is a non-porous support or a porous support

3. The kit of claim 2, wherein AMSS is selected from the group consisting of a bead, a nanodisk, a microdisk, a film, a rod, a nanoparticle, or a microparticle.

4. The kit of claim 2, wherein the AMSS is etched for structure.

5. The kit of claim 2, wherein the AMSS comprises a magnetic bead

6. The kit of claim 2, wherein the AMSS comprises an iron oxide nanoparticle.

7. The kit of claim 1, further comprising an internal reference standard.

8. The kit of claim 7, wherein the internal reference standard comprises isotopically labeled.

9. The kit of claim 7, wherein the reference standard comprises a mass spectrum peak corresponding to a disease-specific biomarker.

10. The kit of claim 9, wherein the mass spectrum peak corresponding to a disease-specific biomarker comprises:
a m/z peak at 1594 is indicative of a disease-specific target antigen TDAATLAQEAGNFER (SEQ ID NO: 1) associated with co-infection by Mycobacterium tuberculosis complex subspecies and one or more of M. kansasii, M. marinum, and M. ulcerans infection;
a m/z peak at 1901 (ESAT-6) is indicative of a disease-specific target antigen WDATATELNNALONLAR (SEQ ID NO: 3) associated with infection by Mycobacterium tuberculosis complex species;
a m/z peak at 2004 is indicative of a disease-specific target antigen TQIDQVESTAGSLQGQWR (SEQ ID NO: 2) associated with infection by Mycobacterium tuberculosis complex subspecies;
a m/z peak at 2032 is indicative of a disease-specific target antigen associated with infection by M. kansasii;
or a combination thereof.

11. The kit of claim 9, wherein the mass spectrum peak corresponding to a disease-specific biomarker comprises:
a m/z peak at 1463 Da is indicative of a disease-specific peptide ETINEEAAEWDR (SEQ ID NO: 5) of HIV-1 infection;
a m/z peak at 1448 Da is indicative of a disease-specific peptide DTINEEAAEWDR (SEQ ID NO: 6) of HIV-1 infection;
a m/z peak at 1294 Da is indicative of a disease-specific peptide MYSPVSILDIR (SEQ ID NO: 9) of HIV-1 infection;
a m/z peak at 1296 Da is indicative of a disease-specific peptide MYSPTSILDIR (SEQ ID NO: 7) of HIV-1 infection;
or a combination thereof.

12. The kit of claim 9, wherein the mass spectrum peak corresponding to a disease-specific biomarker comprises:
a m/z peak at 1322 Da is indicative of a disease specific peptide MYNPTNILDIK (SEQ ID NO: 10) of HIV-2 infection;
a m/z peak at 958 Da is indicative of a disease specific peptide AEQTDPAVK (SEQ ID NO: 11) indicative of HIV-2 infection;
or a combination thereof.

13. The kit of claim 1, wherein the disease or disorder is caused by an infectious pathogen comprising a bacterium, a fungus, or a virus.

14. The kit of claim 13, wherein the bacterial disease or disorder comprises tuberculosis, nontuberculous mycobacterial (NMT) disease, or gut microbial perturbations.

15. The kit of claim 13, wherein the viral disease or disorder comprises a human immunodeficiency virus (HIC) disease, and Ebola.

16. The kit of claim 1, wherein the biomarker is a disease-specific biomarker comprising a disease-specific antigen, a disease-specific protein, a disease specific-peptide, a fragment thereof, or a combination thereof.

17. The kit of claim 16, wherein the disease-specific biomarker is a peptide Mycobacterium peptide comprising the sequence TDAATLAQEAGNFER (SEQ ID NO: 1), TQIDQVESTAGSLQGQWR (SEQ ID NO:2), WDATATELNNALQNLAR (SEQ ID NO:3), TQIDQVESTAASLQAQWR (SEQ ID NO:4), a fragment thereof, or a combination thereof.

18. The kit of claim 16, wherein the disease-specific biomarker is a HIV-1 specific peptide comprising the sequence ETINEEAAEWDR (SEQ ID NO: 5), DTINEEAAEWDR (SEQ ID NO: 6), MYSPTSILDIR (SEQ ID NO: 7), MYSPVSILDIK (SEQ ID NO: 8), MYSPVSILDIR (SEQ ID NO: 9), a fragment thereof, or a combination thereof.

19. The kit of claim 16, wherein the disease-specific biomarker is a HIV-2 specific peptide comprising the sequence MYNPTNILDIK (SEQ ID NO: 10), AEQTDPAVK (SEQ ID NO: 11), a fragment thereof, or a combination thereof.

20. The kit of claim 1, wherein the disease or disorder biomarker is detected by a mass spectrometry-based analytical technique.
